(19) United States
(12) Patent Application Publication
(10) Pub. No.: US 2024/0175012 A1
(43) Pub. Date: May 30, 2024

(54) GUIDE RNAS AND COMPOSITIONS FOR EDITING HUNTINGTIN GENE, AND METHODS RELATED THERETO

(71) Applicants: THE UNIVERSITY OF BRITISH COLUMBIA, Vancouver (CA); INCISIVE GENETICS, INC, Vancouver (CA)

(72) Inventors: Blair LEAVITT, Vancouver (CA); Austin HILL, Vancouver (CA); Pamela WAGNER, Vancouver (CA)

(21) Appl. No.: 18/282,711

(22) PCT Filed: Mar. 18, 2022
(86) PCT No.: PCT/IB2022/052491

§ 371 Date: Sep. 18, 2023

Related U.S. Application Data
(60) Provisional application No. 63/164,404, filed on Mar. 22, 2021.
(60) Provisional application No. 63/163,692, filed on Mar. 19, 2021.

(51) Int. Cl. C12N 15/11; A61K 48/00; A61P 25/28; C12N 9/22; C12N 15/10; C12N 15/63

(52) U.S. Cl. CPC C12N 15/11; A61K 48/005; A61P 25/28; C12N 9/22; C12N 15/102; C12N 15/63; C12N 2310/20

(57) ABSTRACT

gRNAs, combinations of gRNAs, polynucleotides and vectors encoding, RNPs, and compositions for effecting CRISPR-mediated gene editing of the huntingtin gene are provided. Also provided are methods for effecting CRISPR-mediated gene editing of the huntingtin gene and/or for preventing, ameliorating, slowing the progression of, and/or treating HD or HD symptoms.

### RELATED APPLICATIONS

This application claims priority to U.S. Provisional Application Nos. 63/163,692 and 63/164,404 filed on Mar. 19, 2021, and Mar. 22, 2021, respectively, entitled “COMPOSITIONS AND SYSTEMS RELATING TO TRANSFECTION-COMPETENT VESICLES CONTAINING NUCLEASES COMPLEXED TO MULTIPLE DIFFERENT GUIDE RNAS, AND METHODS RELATED THERETO”, the contents of which are incorporated by reference in their entirety herein.

### BACKGROUND

There is a need for devices, systems, methods, etc., for treatments that lessen, or even eliminate symptoms associated with genetically caused and/or related diseases and conditions. In this application, Huntington's Disease (“HD”, also known as “Huntington's chorea”) will be used as an exemplary case. HD is a serious disease that can severely harm its victims. More specifically, Huntington's disease is a neurodegenerative disease that is mostly inherited. The earliest symptoms are often subtle problems with mood or mental abilities, often followed by a general lack of coordination and an unsteady gait. As the disease advances, uncoordinated, involuntary body movements known as chorea become more apparent. Physical abilities gradually worsen until coordinated movement becomes difficult and the person is unable to talk. Mental abilities generally decline into dementia. Symptoms usually begin between 30 and 50 years of age but can start at any age. Some estimates indicate that HD affects approximately 1 in 10,000 people in Canada, and about 1 in 70,000 worldwide.

Turning to a more scientific discussion, HD is a progressive, neurodegenerative disorder caused by an expanded trinucleotide CAG repeat in the huntingtin gene. Normal forms of the gene, also known as “wild-type (WT)”, contain 10-35 CAG repeats, whereas the mutated disease-causing forms contain 36 or more. Longer trinucleotide expansions correlate to an earlier onset of disease. The huntingtin gene is critical to development, as embryos lacking functional protein are not viable. The mutant form of the gene, with 36 or more CAG repeats, confers a toxic gain-of-function to the huntingtin protein via encoding a long polyglutamine (polyQ) tract within exon 1 of the gene. This repeat expansion is inherited in an autosomal dominant pattern. Children of affected parents have a 50% chance of inheriting the disease-causing mutation.

The huntingtin protein is large, around 340 kDa, which has complicated investigation of its function within the cell. HD therapeutics typically are directed to symptomatic treatment, but do nothing to halt or slow HD disease progression. In order to more effectively treat genetic diseases, the therapeutic should target the affected genes or gene products (such as the huntingtin protein).

CRISPR has recently emerged as a powerful tool used for gene editing. It can be used to target mutations in DNA. It has the potential to fix mutations or knockout genes that cause disease. The functional CRISPR machinery involves the ribonucleoprotein (RNP) complex, which is comprised of a nuclease protein and a guide RNA (gRNA). Suitable nucleases can be one of various enzymes, including Cas9, Cas10, Cas11, Cas12, or Cpf1. Other nucleases, including both endonucleases and exonucleases are also suitable.

Effectively introducing such HD potential therapeutics into affected cells and genes has been troubled, for example by undesirable side effects and failures to achieve adequate therapeutic effects.

Thus, there has gone unmet a need for compositions, systems, methods, etc., for treatments that lessen, or even eliminate symptoms associated with genetically-caused and/or related diseases and conditions, for example HD symptoms, HD causes, and/or HD transmissibility to children.

The present systems and methods, etc., provide solutions to one or more of these needs, and/or one or more other advantages.

### SUMMARY

Some aspects of the present disclosure relate to gRNAs, combinations of gRNAs, RNPs, and compositions, which may be used for effecting CRISPR-mediated gene editing of the huntingtin gene.

In one aspect, the present disclosure provides isolated gRNAs, which may be used for editing the huntingtin gene, such as the human huntingtin gene, HTT.

In some embodiments, a gRNA according to the present disclosure may allow for CRISPR-mediated gene editing of exon 5 of HTT or its proximate gene segment.

In certain embodiments, the gRNA may comprise a targeting sequence comprising at least or consisting of 17 nucleotides, optionally 17, 18, 19, 20, 21, 22, 23, 24, 25, 26, 27, 28, 29, or 30 nucleotides.

In certain embodiments, the targeting sequence may comprise or consist of (i) (i-1) a sequence of at least 17 consecutive nucleotides, optionally 17, 18, 19, 20, 21, 22, 23, 24, 25, 26, 27, 28, 29, or 30 consequent nucleotides, contained in exon 5 or its proximate gene segment, for example in one or more of SEQ ID NO: 200, 201, and/or 202, or SEQ ID NO: 205, 206, and/or 207 (reverse complement of SEQ ID NO: 200, 201, and/or 202 respectively), and/or (i-2) a sequence of: (a) SEQ ID NO: 4, 210, 211, 212, or 213, (b) SEQ ID NO: 5, 220, 221, 222, or 223, or (c) SEQ ID NO: 6, 230, 231, 232, or 233.

In certain embodiments, the targeting sequence may comprise or consist of a sequence of at least 17 nucleotides comprising one or more mutations, optionally one, two, three, four, or five mutations, said mutations relative to the at least 17 consecutive nucleotides, optionally 17, 18, 19, 20, 21, 22, 23, 24, 25, 26, 27, 28, 29, or consequent nucleotides, of (i), optionally wherein the one or more mutations are at any nucleotide position(s) or are at position(s) other than the 4th to the 7th nucleotide positions from the 3′-end of the at least 17 consecutive nucleotides.

In certain embodiments, the targeting sequence may comprise or consist of a sequence of at least 17 nucleotides which comprises at least 85, 90, 95, 96, 97, 98 or 99% sequence identity to the sequence of at least 17 consecutive nucleotides, optionally 17, 18, 19, 20, 21, 22, 23, 24, 25, 26, 27, 28, 29, or 30 consecutive nucleotides, of (i).

In some instances, the at least 17 consecutive nucleotides (as referred to in (i-1) above may be immediately upstream of a protospacer adjacent motif (PAM) or protospacer flanking site (PFS) of a CRISPR-associated (Cas) endonuclease in one or more of SEQ ID NO: 200, 201, 202, 205, 206, and/or 207. In some instances, the Cas endonuclease may be Cas9, optionally Streptococcus pyogenes Cas9 (SpCas9). In some instances, the PAM sequence may be 5′-NGG-3′, wherein N represents any nucleotide. In some instances, the at least 17 consecutive nucleotides (as referred to in (i-1) above may be immediately downstream of the PAM or PFS of a Cas endonuclease in one or more of SEQ ID NO: 200, 201, 202, 205, 206, and/or 207. In some instances, the Cas endonuclease may be Cpf1. In some instances, the PAM sequence is 5′-TTTN-3′ wherein N represents any nucleotide.

In certain embodiments, when the gRNA may be complexed with a Cas endonuclease, the targeting sequence may guide the Cas endonuclease to allow for cleavage of exon 5 of HTT or within 30 nucleotides upstream or downstream of the exon 5. In some instances, the cleavage may be within one or more of SEQ ID NOS: 200, 201, 202, 205, 206, and/or 207, optionally at any one or more of the following positions: (i-1) between the 9^{th} and 10^{th} nucleotides from the 5′ end of SEQ ID NO: 200, (i-2) between the 55^{th} and 56^{th} nucleotides from the 5′ end of SEQ ID NO: 200, (i-3) between the 72^{th} and 73^{th} nucleotides from the 5′ end of SEQ ID NO: 200, (i-4) between the 9^{th} and 10^{th} nucleotides from the 3′ end of SEQ ID NO: 205, (i-5) between the 55^{th} and 56^{th} nucleotides from the 3′ end of SEQ ID NO: 205, (i-6) between the 72^{th} and 73^{th} nucleotides from the 3′ end of SEQ ID NO: 200. In some instances, the Cas endonuclease is Cas 9, Cas3, Cas8a2, Cas8b, Cas8c, Cas10, Cas11, Cas12, Cas12a or Cpf1, Cas13, Cas13a, C2c1, C2c3, or C2c2, further optionally Cas9 or Cpf1, yet further optionally SpCas9.

In some embodiments, a gRNA according to the present disclosure may allow for CRISPR-mediated gene editing of exon 8 of HTT or its proximate gene segment.

In certain embodiments, the gRNA may comprise a targeting sequence comprising at least or consisting of 17 nucleotides, optionally 17, 18, 19, 20, 21, 22, 23, 24, 25, 26, 27, 28, 29, or 30 nucleotides.

In certain embodiments, the targeting sequence may comprise or consist of (i) (i-1) a sequence of at least 17 consecutive nucleotides, optionally 17, 18, 19, 20, 21, 22, 23, 24, 25, 26, 27, 28, 29, or 30 consequent nucleotides, contained in exon 8 or its proximate gene segment, for example in one or more of SEQ ID NO: 300, 301, and/or 302, or SEQ ID NO: 305, 306, and/or 307 (reverse complement of SEQ ID NO: 300, 301, and/or 302 respectively), and/or (i-2) a sequence of: (a) SEQ ID NO: 7, 310, 311, 312, or 313, (b) SEQ ID NO: 8, 320, 321, 322, or 323, or (c) SEQ ID NO: 9, 330, 331, 332, or 333.

In certain embodiments, the targeting sequence may comprise or consist of a sequence of at least 17 nucleotides comprising one or more mutations, optionally one, two, three, four, or five mutations, said mutations relative to the at least 17 consecutive nucleotides, optionally 17, 18, 19, 20, 21, 22, 23, 24, 25, 26, 27, 28, 29, or consequent nucleotides, of (i), optionally wherein the one or more mutations are at any nucleotide position(s) or are at position(s) other than the 4^{th} to the 7^{th} nucleotide positions from the 3′-end of the at least 17 consecutive nucleotides.

In certain embodiments, the targeting sequence may comprise or consist of a sequence of at least 17 nucleotides which comprises at least 85, 90, 95, 96, 97, 98 or 99% sequence identity to the sequence of at least 17 consecutive nucleotides, optionally 17, 18, 19, 20, 21, 22, 23, 24, 25, 26, 27, 28, 29, or 30 consecutive nucleotides, of (i).

In some instances, the at least 17 consecutive nucleotides (as referred to in (i-1) above may be immediately upstream of a PAM or PFS of a Cas endonuclease in one or more of SEQ ID NO: 300, 301, 302, 305, 306, and/or 307. In some instances, the Cas endonuclease may be Cas9, optionally SpCas9. In some instances, the PAM sequence may be 5′-NGG-3′, wherein N represents any nucleotide. In some instances, the at least 17 consecutive nucleotides (as referred to in (i-1) above) may be immediately downstream of the PAM or PFS of a Cas endonuclease in one or more of SEQ ID NO: 300, 301, 302, 305, 306, and/or 307. In some instances, the Cas endonuclease may be Cpf1. In some instances, the PAM sequence is 5′-TTTN-3′ wherein N represents any nucleotide.

In certain embodiments, when the gRNA may be complexed with a Cas endonuclease, the targeting sequence may guide the Cas endonuclease to allow for cleavage of exon 8 of HTT or within 30 nucleotides upstream or downstream of the exon 8. In some instances, the cleavage may be within one or more of SEQ ID NOS: 300, 301, 302, 305, 306, and/or 307, optionally at any one or more of the following positions: (i-1) between the 15^{th} and 16^{th} nucleotides from the 5′ end of SEQ ID NO: 300, (i-2) between the 69^{th} and 70^{th} nucleotides from the 5′ end of SEQ ID NO: 300, (i-3) between the 112^{th} and 113^{th} nucleotides from the 5′ end of SEQ ID NO: 300, (i-4) between the 15^{th} and 16^{th} nucleotides from the 3′ end of SEQ ID NO: 305, (i-5) between the 69^{th} and 70^{th} nucleotides from the 3′ end of SEQ ID NO: 305, (i-6) between the 112^{th} and 113^{th} nucleotides from the 3′ end of SEQ ID NO: 305. In some instances, the Cas endonuclease is Cas 9, Cas3, Cas8a2, Cas8b, Cas8c, Cas10, Cas11, Cas12, Cas12a or Cpf1, Cas13, Cas13a, C2c1, C2c3, or C2c2, or is Cas9 or Cpf1, or is SpCas9.

In some embodiments, a gRNA according to the present disclosure may allow for CRISPR-mediated gene editing of exon 31 of HTT or its proximate gene segment.

In certain embodiments, the gRNA may comprise a targeting sequence comprising at least or consisting of 17 nucleotides, optionally 17, 18, 19, 20, 21, 22, 23, 24, 25, 26, 27, 28, 29, or 30 nucleotides.

In certain embodiments, the targeting sequence may comprise or consist of (i) (i-1) a sequence of at least 17 consecutive nucleotides, optionally 17, 18, 19, 20, 21, 22, 23, 24, 25, 26, 27, 28, 29, or 30 consequent nucleotides, contained in exon 31 or its proximate gene segment, for example in one or more of SEQ ID NO: 400, 401, and/or 402, or SEQ ID NO: 405, 406, and/or 407 (reverse complement of SEQ ID NO: 400, 401, and/or 402 respectively), and/or (i-2) a sequence of: (a) SEQ ID NO: 1, 410, 411, 412, or 413, (b) SEQ ID NO: 2, 420, 421, 422, or 423, (c) SEQ ID NO: 3, 430, 431, 432, or 433, (d) SEQ ID NO: 440, 441, 442, or 443, (e) SEQ ID NO: 450, 451, 452, or 453.

In certain embodiments, the targeting sequence may comprise or consist of a sequence of at least 17 nucleotides comprising one or more mutations, optionally one, two, three, four, or five mutations, said mutations relative to the at least 17 consecutive nucleotides, optionally 17, 18, 19, 20, 21, 22, 23, 24, 25, 26, 27, 28, 29, or consequent nucleotides, of (i), optionally wherein the one or more mutations are at any nucleotide position(s) or are at position(s) other than the 4^{th} to the 7^{th} nucleotide positions from the 3′-end of the at least 17 consecutive nucleotides.

In certain embodiments, the targeting sequence may comprise or consist of a sequence of at least 17 nucleotides which comprises at least 85, 90, 95, 96, 97, 98 or 99% sequence identity to the sequence of at least 17 consecutive nucleotides, optionally 17, 18, 19, 20, 21, 22, 23, 24, 25, 26, 27, 28, 29, or 30 consecutive nucleotides, of (i).

In some instances, the at least 17 consecutive nucleotides (as referred to in (i-1) above) may be immediately upstream of a PAM or PFS of a Cas endonuclease in one or more of SEQ ID NO: 400, 401, 402, 405, 406, and/or 407. In some instances, the Cas endonuclease may be Cas9, optionally SpCas9. In some instances, the PAM sequence may be 5′-NGG-3′, wherein N represents any nucleotide. In some instances, the at least 17 consecutive nucleotides (as referred to in (i-1) above) may be immediately downstream of the PAM or PFS of a Cas endonuclease in one or more of SEQ ID NO: 400, 401, 402, 405, 406, and/or 407. In some instances, the Cas endonuclease may be Cpf1. In some instances, the PAM sequence is 5′-TTTN-3′ wherein N represents any nucleotide.

In certain embodiments, when the gRNA may be complexed with a Cas endonuclease, the targeting sequence may guide the Cas endonuclease to allow for cleavage of exon 31 of HTT or within 30 nucleotides upstream or downstream of the exon 31. In some instances, the cleavage may be within one or more of SEQ ID NOS: 400, 401, 402, 405, 406, and/or 407, optionally at any one or more of the following positions: (i-1) between the 36^{th} and 37^{th} nucleotides from the 5′ end of SEQ ID NO: 400, (i-2) between the 106^{th} and 107^{th} nucleotides from the 5′ end of SEQ ID NO: 400, (i-3) between the 185^{th} and 186^{th} nucleotides from the 5′ end of SEQ ID NO: 400, (i-4) between the 112^{th} and 113^{t}h nucleotides from the 5′ end of SEQ ID NO: 400, (i-5) between the 101^{th} and 102^{th} nucleotides from the 5′ end of SEQ ID NO: 400, (i-6) between the 36^{th} and 37^{th} nucleotides from the 3′ end of SEQ ID NO: 405, (i-7) between the 106^{th} and 107^{th} nucleotides from the 3′ end of SEQ ID NO: 405, (i-8) between the 185^{th} and 186^{th} nucleotides from the 3′ end of SEQ ID NO: 405, (i-9) between the 112^{th} and 113^{th} nucleotides from the 3′ end of SEQ ID NO: 405, (i-10) between the 101^{th} and 102^{th} nucleotides from the 3′ end of SEQ ID NO: 405. In some instances, the Cas endonuclease is Cas 9, Cas3, Cas8a2, Cas8b, Cas8c, Cas10, Cas11, Cas12, Cas12a or Cpf1, Cas13, Cas13a, C2c1, C2c3, or C2c2, or is Cas9 or Cpf1 or is SpCas9.

In some embodiments relating to one or more gRNAs such as those described above, the gRNA may be a single guide RNA (sgRNA) comprising in a single strand: (i) a CRISPR RNA (crRNA) sequence comprising the targeting sequence and a crRNA backbone sequence, and (ii) a trans-activating CRISPR RNA (tracrRNA) sequence. In some instances, the crRNA sequence and the tracrRNA sequence may be linked via a linker optionally comprising SEQ ID NO: 109. In some instances, the gRNA may comprise the targeting sequence followed by, optionally immediately followed by, a sgRNA backbone sequence of any of SEQ ID NOS: 111-114. In particular instances, the sgRNA backbone sequence may be followed by one or more uracils, optionally 1-10 uracils.

In some embodiments relating to one or more gRNAs such as those described above, the gRNA may be a dual guide RNA (dgRNA) formed by hybridization between: (i) a crRNA comprising the targeting sequence and a crRNA backbone sequence, and (ii) a tracrRNA. In some instances, the targeting sequence may be followed by, optionally immediately followed by, a sgRNA backbone sequence of SEQ ID NOS: 115 and the tracrRNA may comprise SEQ ID NO: 116. In some instances, the targeting sequence may be followed by, optionally immediately followed by, a sgRNA backbone sequence of SEQ ID NOS: 117 and the tracrRNA may comprise SEQ ID NO: 118.

In some embodiments relating to one or more gRNAs such as those described above, the gRNA may be synthetic or recombinant.

In some embodiments relating to one or more gRNAs such as those described above, the gRNA may be a synthetic sgRNA and may comprise at least one chemical modification. In certain embodiments, the modification may be (i) 2′-O-methylation optionally at the first three and last three bases, and/or (ii) one or more 3′ phosphorothioate bonds, optionally between the first three and last two bases.

In one aspect, the present disclosure provides combinations of isolated gRNAs, which may be for editing the huntingtin gene, such as the human huntingtin gene, HTT.

In some embodiments, a combination of gRNAs may comprise two, three, or more gRNAs for editing exon 5 of HTT or its proximate gene segment. In certain embodiments, such gRNAs may be selected from any of the gRNAs described above for editing exon 5 of HTT or its proximate gene segment. In particular embodiments, a combination of gRNAs may comprise three different gRNAs having the targeting sequences of (a) SEQ ID NO: 4, 210, 211, 212, or 213, (b) SEQ ID NO: 5, 220, 221, 222, or 223, and (c) SEQ ID NO: 6, 230, 231, 232, or 233, respectively.

In some embodiments, a combination of gRNAs may comprise two, three, or more gRNAs for editing exon 8 of HTT or its proximate gene segment. In certain embodiments, such gRNAs may be selected from any of the gRNAs described above for editing exon 8 of HTT or its proximate gene segment. In particular embodiments, a combination of gRNAs may comprise three different gRNAs having the targeting sequences of: (a) SEQ ID NO: 7, 310, 311, 312, or 313, (b) SEQ ID NO: 8, 320, 321, 322, or 323, and (c) SEQ ID NO: 9, 330, 331, 332, or 333, respectively.

In some embodiments, a combination of gRNAs may comprise two, three, or more gRNAs for editing exon 31 of HTT or its proximate gene segment. In certain embodiments, such gRNAs may be selected from any of the gRNAs described above for editing exon 31 of HTT or its proximate gene segment. In particular embodiments, a combination of gRNAs may comprise three different gRNAs having the targeting sequences of: (a) SEQ ID NO: 1, 410, 411, 412, or 413, (b) SEQ ID NO: 2, 420, 421, 422, or 423, and (c) SEQ ID NO: 3, 430, 431, 432, or 433, respectively. In some alternative embodiments, a combination of gRNAs may comprise three different gRNAs having the targeting sequences of: (a) SEQ ID NO: 1, 410, 411, 412, or 413, (b) SEQ ID NO: 440, 441, 442, or 443, or 450, 451, 452, or 453, and (c) SEQ ID NO: 3, 430, 431, 432, or 433, respectively.

In some embodiments, a combination of gRNAs may comprise two or more gRNAs for editing at least two exons selected from exons 5, 8, and 31 of HTT. In certain embodiments, such gRNAs may be selected from any of the gRNAs described above for editing exon 5, 8, or 31 of HTT or its proximate gene segment. In certain embodiments, a combination of gRNAs may comprise at least one gRNA for editing exons 5 of HTT, at least one gRNA for editing exons 8 of HTT, and at least one gRNA for editing exons 31 of HTT. In particular embodiments, a combination of gRNAs may comprise: (I) three different gRNAs having the targeting sequences of: (a) SEQ ID NO: 4, 210, 211, 212, or 213, (b) SEQ ID NO: 5, 220, 221, 222, or 223, and (c) SEQ ID NO: 6, 230, 231, 232, or 233, respectively; (II) three different gRNAs having the targeting sequences of: (a) SEQ ID NO: 7, 310, 311, 312, or 313, (b) SEQ ID NO: 8, 320, 321, 322, or 323, and (c) SEQ ID NO: 9, 330, 331, 332, or 333, respectively; and (III) three different gRNAs having the targeting sequences of: (a) SEQ ID NO: 1, 410, 411, 412, or 413, (b) SEQ ID NO: 2, 420, 421, 422, or 423, and (c) SEQ ID NO: 3, 430, 431, 432, or 433, respectively.

In one aspect, the present disclosure provides a polynucleotide or polynucleotides encoding any one or more of the isolated gRNAs described above.

In some embodiments, the polynucleotide or polynucleotides may encode one or more of the gRNAs for editing exon 5 of HTT or its proximate gene segment described above. In some embodiments, the polynucleotide or polynucleotides may encode one or more of the gRNAs for editing exon 8 of HTT or its proximate gene segment described above. In some embodiments, the polynucleotide or polynucleotides may encode one or more of the gRNAs for editing exon 31 of HTT or its proximate gene segment described above. In some embodiments, the polynucleotide or polynucleotides may encode multiple gRNAs for editing at least two of exons 5, 8, and 31 of HTT or the proximate gene segments described above.

In one aspect, the present disclosure provides a vector or vectors comprising any of the polynucleotide or polynucleotides described above. In some embodiments, the vector or vectors may be individually selected from plasmids, RNA replicons, virus-like particles (VLPs), and viral vectors, optionally retroviral, lentiviral, or adenoviral vectors.

In one aspect, the present disclosure provides RNPs, which may be for editing the huntingtin gene, such as the human huntingtin gene, HTT.

In some embodiments, a RNP may comprise (a) any one or more isolated gRNAs described above; which is/are complexed with (b) a Cas endonuclease.

In certain embodiments, the Cas endonuclease may be selected from the group consisting of Cas9, Cas3, Cas8a2, Cas8b, Cas8c, Cas10, Cas11, Cas12, Cas12a or Cpf1, Cas13, Cas13a, C2c1, C2c3, and C2c2. In certain embodiments, the Cas endonuclease may be a class 2 Cas endonuclease, optionally a type II, type V, or type VI Cas nuclease. In certain embodiments, the Cas endonuclease may be Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, eSpCas9, HypaCas9, FokI-Fused dCas9, xCas9, SpRY (variant of SpCas9), and SpG (variant of SpCas9). In certain embodiments, the Cas endonuclease may be Cas9, optionally comprising any one of SEQ ID NOS: 600-611.

In certain embodiments, the RNP may be formed by mixing at an approximately equimolar ratio (I) a solution comprising the one or more isolated gRNAs, optionally wherein the pH of the solution is about 6 to 8, about 6.5 to 7.5, further optionally about 7, and (II) a solution comprising the Cas endonuclease, optionally wherein the pH of the solution is about 6 to 8, about 6.5 to 7.5, further optionally about 7, further optionally wherein the mixing is for about 5 minutes.

In certain embodiments, the RNPs may be a combination of two or more RNPs. In some cases, the RNPs may comprise two or more RNPs for editing exon 5 of HTT or its proximate gene segment. In some cases, the RNPs may comprise two or more RNPs for editing exon 8 of HTT or its proximate gene segment. In some cases, the RNPs may comprise two or more RNPs for editing exon 31 of HTT or its proximate gene segment. In some cases, the RNPs may comprise multiple RNPs for editing at least two exons of exons 5, 8, and 31 of HTT or the proximate gene segments. In some cases, the RNPs may comprise: (I) at least one RNP (e.g., three RNPs having different targeting sequences) for editing exon 5 of HTT or its proximate gene segment; (II) at least one RNP (e.g., three RNPs having different targeting sequences) for editing exon 8 of HTT or its proximate gene segment; and (III) at least one RNP (e.g., three RNPs having different targeting sequences) for editing exon 31 of HTT or its proximate gene segment.

In one aspect, the present disclosure provides compositions comprising: (A) a pharmaceutically acceptable carrier; and (B) any one or more RNPs described above; and (C) optionally one or more template DNAs.

In some embodiments, the one or more RNPs may comprise one or more of the following (I-1)-(I-3):

(I-1) a first isolated gRNA for editing exon 5 of HTT or its proximate gene segment described above, which may comprise a first targeting sequence comprising or consisting of: (i) a sequence of SEQ ID NO: 210, 211, 212, or 213, (ii) a sequence comprising one or more mutations, optionally one, two, three, four, or five mutations, relative to SEQ ID NO: 210, 211, 212, or 213, optionally wherein the one or more mutations are at any nucleotide position(s) or are at position(s) other than the 4^{th} to the 7^{th} nucleotide positions from the 3′-end of SEQ ID NO: 210, 211, 212, or 213, or (iii) a sequence comprising at least 85, 90, 95, 96, 97, 98 or 99% sequence identity to SEQ ID NO: 210, 211, 212, or 213;

(I-2) a second isolated gRNA for editing exon 5 of HTT or its proximate gene segment described above, which may comprise a second targeting sequence comprising or consisting of: (i) a sequence of SEQ ID NO: 220, 221, 222, or 223, (ii) a sequence comprising one or more mutations, optionally one, two, three, four, or five mutations, relative to SEQ ID NO: 220, 221, 222, or 223, optionally wherein the one or more mutations are at any nucleotide position(s) or are at position(s) other than the 4^{th} to the 7^{th} nucleotide positions from the 3′-end of SEQ ID NO: 220, 221, 222, or 223, or (iii) a sequence comprising at least 85, 90, 95, 96, 97, 98 or 99% sequence identity to SEQ ID NO: 220, 221, 222, or 223; and/or

(I-3) a third isolated gRNA for editing exon 5 of HTT or its proximate gene segment described above, which may comprise a third targeting sequence comprising or consisting of: (i) a sequence of SEQ ID NO: 230, 231, 232, or 233, (ii) a sequence comprising one or more mutations, optionally one, two, three, four, or five mutations, relative to SEQ ID NO: 230, 231, 232, or 233, optionally wherein the one or more mutations are at any nucleotide position(s) or are at position(s) other than the 4^{th} to the 7^{th} nucleotide positions from the 3′-end of SEQ ID NO: 230, 231, 232, or 233, or (iii) a sequence comprising at least 85, 90, 95, 96, 97, 98 or 99% sequence identity to SEQ ID NO: 230, 231, 232, or 233.

In some embodiments, the one or more RNPs may comprise all of: (I-1) the first isolated gRNA which comprises the first targeting sequence of SEQ ID NO: 210, 211, 212, or 213; (I-2) the second isolated gRNA comprising the second targeting sequence of SEQ ID NO: 220, 221, 222, or 223; and (I-3) the third isolated gRNA comprising the third targeting sequence of SEQ ID NO: 230, 231, 232, or 233, and optionally (I-4) one or more isolated gRNAs for editing exon 5 of HTT or its proximate gene segment described above.

In some embodiments, the one or more RNPs may comprise one or more of the following (II-1)-(II-3):

(II-1) a fourth isolated gRNA for editing exon 8 of HTT or its proximate gene segment described above, which may comprise a fourth targeting sequence comprising or consisting of: (i) a sequence of SEQ ID NO: 310, 311, 312, or 313, (ii) a sequence comprising one or more mutations, optionally one, two, three, four, or five mutations, relative to SEQ ID NO: 310, 311, 312, or 313, optionally wherein the one or more mutations are at any nucleotide position(s) or are at position(s) other than the 4^{th} to the 7^{th} nucleotide positions from the 3′-end of SEQ ID NO: 310, 311, 312, or 313, or (iii) a sequence comprising at least 85, 90, 95, 96, 97, 98 or 99% sequence identity to SEQ ID NO: 310, 311, 312, or 313;

(II-2) a fifth isolated gRNA for editing exon 8 of HTT or its proximate gene segment described above, which may comprise a fifth targeting sequence comprising or consisting of: (i) a sequence of SEQ ID NO: 320, 321, 322, or 323, (ii) a sequence comprising one or more mutations, optionally one, two, three, four, or five mutations, relative to SEQ ID NO: 320, 321, 322, or 323, optionally wherein the one or more mutations are at any nucleotide position(s) or are at position(s) other than the 4^{th} to the 7^{th} nucleotide positions from the 3′-end of SEQ ID NO: 320, 321, 322, or 323, or (iii) a sequence comprising at least 85, 90, 95, 96, 97, 98 or 99% sequence identity to SEQ ID NO: 320, 321, 322, or 323; and/or

(II-3) a sixth isolated gRNA for editing exon 8 of HTT or its proximate gene segment described above, which may comprise a sixth targeting sequence comprising or consisting of: (i) a sequence of SEQ ID NO: 330, 331, 332, or 333, (ii) a sequence comprising one or more mutations, optionally one, two, three, four, or five mutations, relative to SEQ ID NO: 330, 331, 332, or 333, optionally wherein the one or more mutations are at any nucleotide position(s) or are at position(s) other than the 4^{th} to the 7^{th} nucleotide positions from the 3′-end of SEQ ID NO: 330, 331, 332, or 333, or (iii) a sequence comprising at least 85, 90, 95, 96, 97, 98 or 99% sequence identity to SEQ ID NO: 330, 331, 332, or 333.

In some embodiments, the one or more RNPs may comprise all of: (II-1) the fourth isolated gRNA which comprises the fourth targeting sequence of SEQ ID NO: 310, 311, 312, or 2313; (II-2) the fifth isolated gRNA comprising the fifth targeting sequence of SEQ ID NO: 320, 321, 322, or 323; and (II-3) the sixth isolated gRNA comprising the sixth targeting sequence of SEQ ID NO: 330, 331, 332, or 333, and optionally (II-4) one or more isolated gRNAs for editing exon 8 of HTT or its proximate gene segment described above.

In some embodiments, the one or more RNPs may comprise one or more of the following (III-1)-(III-4):

(III-1) a seventh isolated gRNA for editing exon 31 of HTT or its proximate gene segment described above, which comprises a seventh targeting sequence comprising or consisting of: (i) a sequence of SEQ ID NO: 410, 411, 412, or 413, (ii) a sequence comprising one or more mutations, optionally one, two, three, four, or five mutations, relative to SEQ ID NO: 410, 411, 412, or 413, optionally wherein the one or more mutations are at any nucleotide position(s) or are at position(s) other than the 4^{th} to the 7^{th} nucleotide positions from the 3′-end of 410, 411, 412, or 413, or (iii) a sequence comprising at least 85, 90, 95, 96, 97, 98 or 99% sequence identity to SEQ ID NO: 410, 411, 412, or 413;

(III-2) an eighth isolated gRNA for editing exon 31 of HTT or its proximate gene segment described above, which comprises an eighth targeting sequence comprising or consisting of: (i) a sequence of SEQ ID NO: 420, 421, 422, or 423, (ii) a sequence comprising one or more mutations, optionally one, two, three, four, or five mutations, relative to SEQ ID NO: 420, 421, 422, or 423, optionally wherein the one or more mutations are at any nucleotide position(s) or are at position(s) other than the 4^{th} to the 7^{th} nucleotide positions from the 3′-end of SEQ ID NO: 420, 421, 422, or 423, or (iii) a sequence comprising at least 85, 90, 95, 96, 97, 98 or 99% sequence identity to SEQ ID NO: 420, 421, 422, or 423;

(III-3) a ninth, isolated gRNA for editing exon 31 of HTT or its proximate gene segment described above, which comprises a ninth, targeting sequence comprising or consisting of: (i) a sequence of SEQ ID NO: 430, 431, 432, or 433, (ii) a sequence comprising one or more mutations, optionally one, two, three, four, or five mutations, relative to SEQ ID NO: 430, 431, 432, or 433, optionally wherein the one or more mutations are at any nucleotide position(s) or are at position(s) other than the 4^{th} to the 7^{th} nucleotide positions from the 3′-end of SEQ ID NO: 430, 431, 432, or 433, or (iii) a sequence comprising at least 85, 90, 95, 96, 97, 98 or 99% sequence identity to SEQ ID NO: 430, 431, 432, or 433; and/or

(III-4) a tenth isolated gRNA for editing exon 31 of HTT or its proximate gene segment described above, which comprises a tenth targeting sequence comprising or consisting of: (i) a sequence of SEQ ID NO: 440, 441, 442, or 443 or 450, 451, 452, or 453, (ii) a sequence comprising one or more mutations, optionally one, two, three, four, or five mutations, relative to SEQ ID NO: 440, 441, 442, or 443 or 450, 451, 452, or 453, optionally wherein the one or more mutations are at any nucleotide position(s) or are at position(s) other than the 4^{th} to the 7^{th} nucleotide positions from the 3′-end of SEQ ID NO: 440, 441, 442, or 443 or 450, 451, 452, or 453, or (iii) a sequence comprising at least 85, 90, 95, 96, 97, 98 or 99% sequence identity to SEQ ID NO: 440, 441, 442, or 443 or 450, 451, 452, or 453.

In some embodiments, the one or more RNPs may comprise all of: (III-1) the seventh isolated gRNA which comprises the seventh targeting sequence of SEQ ID NO: 410, 411, 412, or 413, (III-2) the eighth isolated gRNA comprising the eighth targeting sequence of SEQ ID NO: 420, 421, 422, or 423, and (III-3) the ninth, isolated gRNA comprising the ninth, targeting sequence of SEQ ID NO: 430, 431, 432, or 433, and optionally (III-5) one or more isolated gRNAs for editing exon 31 of HTT or its proximate gene segment described above.

In some embodiments, the one or more RNPs may comprise all of: (III-1) the seventh isolated gRNA which comprises the seventh targeting sequence of SEQ ID NO: 410, 411, 412, or 413, (III-3) the ninth, isolated gRNA comprising the ninth, targeting sequence of SEQ ID NO: 430, 431, 432, or 433, and (III-4) the tenth isolated gRNA comprising the tenth targeting sequence of SEQ ID NO: 440, 441, 442, or 443 or 450, 451, 452, or 453, and optionally (III-5) one or more isolated gRNAs for editing exon 31 of HTT or its proximate gene segment described above.

In some embodiments, the pharmaceutically acceptable carrier may comprise a lipid-based transfection competent vesicle (TCV).

In certain embodiments, the one or more template DNAs, if present, and/or the one or more RNPs may be encapsulated in the TCV For example, the encapsulation may be performed by (i) providing an aqueous solution comprising the TCV, optionally wherein the aqueous solution and (ii) mixing the one or more template DNAs, if present, and/or one or more of the one or more RNPs with the aqueous solution.

In some cases, in (i), the aqueous solution may have the pH of about 3 to about 8, about 4 to about 7.5, about 3.5 to 4.5, or about 4, optionally wherein said aqueous solution comprises an acetate buffer, optionally an about 25-100 mM or about 25 mM acetate buffer. In some cases, in (i), the aqueous solution may be: substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS); optionally substantially, essentially, or entirely free of organic solvents and/or detergents; further optionally substantially, essentially, or entirely free of destabilizing agents.

In some cases, in (ii), the mixing may comprise gentle mixing (optionally repeated manual reciprocation of the TCV-generating fluid in a pipette), mixing using a staggered herringbone micromixer (SHM), T-junction mixing, or extrusion, and optionally wherein the mixing time is about 0.1 second to about 20 minutes. In some cases, in (ii), the mixing may be performed: substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS); optionally substantially, essentially, or entirely free of organic solvents and/or detergents; further optionally substantially, essentially, or entirely free of destabilizing agents. In some cases, in (ii), when more than one RNPs are encapsulated in the TCV, the mixing may comprise mixing an equimolar ratio of the more than one RNPs with the aqueous solution. In some cases, in (ii), the lipid-based TCV and the RNP(s) may be mixed at an about 100:1 to about 10000:1 molar ratio, optionally at about 500:1 to about 5000:1 molar ratio, further optionally at about 1000:1 to about 2000:1 molar ratio, of the total lipid components of the lipid-based TCV: the RNP(s).

In certain embodiments, the one or more template DNAs, if present, may be co-encapsulated with or separately encapsulated from one or more of the one or more RNPs

In one aspect, the present disclosure provides compositions comprising: (A) a pharmaceutically acceptable carrier; (B) (a) any one or more isolated gRNAs described above or one or more polynucleotides encoding the one or more isolated gRNAs, and (b) a Cas endonuclease or a polynucleotide encoding a Cas endonuclease; and (C) optionally one or more template DNAs or one or more polynucleotides encoding one or more template DNAs.

In some embodiments, In some embodiments, the one or more RNPs may comprise one or more of the following (I-1)-(I-3):

(I-1) a first isolated gRNA for editing exon 5 of HTT or its proximate gene segment described above, which may comprise a first targeting sequence comprising or consisting of: (i) a sequence of SEQ ID NO: 210, 211, 212, or 213, (ii) a sequence comprising one or more mutations, optionally one, two, three, four, or five mutations, relative to SEQ ID NO: 210, 211, 212, or 213, optionally wherein the one or more mutations are at any nucleotide position(s) or are at position(s) other than the 4^{th} to the 7^{th} nucleotide positions from the 3′-end of SEQ ID NO: 210, 211, 212, or 213, or (iii) a sequence comprising at least 85, 90, 95, 96, 97, 98 or 99% sequence identity to SEQ ID NO: 210, 211, 212, or 213;

(I-2) a second isolated gRNA for editing exon 5 of HTT or its proximate gene segment described above, which may comprise a second targeting sequence comprising or consisting of: (i) a sequence of SEQ ID NO: 220, 221, 222, or 223, (ii) a sequence comprising one or more mutations, optionally one, two, three, four, or five mutations, relative to SEQ ID NO: 220, 221, 222, or 223, optionally wherein the one or more mutations are at any nucleotide position(s) or are at position(s) other than the 4^{th} to the 7^{th} nucleotide positions from the 3′-end of SEQ ID NO: 220, 221, 222, or 223, or (iii) a sequence comprising at least 85, 90, 95, 96, 97, 98 or 99% sequence identity to SEQ ID NO: 220, 221, 222, or 223; and/or

(I-3) a third isolated gRNA for editing exon 5 of HTT or its proximate gene segment described above, which may comprise a third targeting sequence comprising or consisting of: (i) a sequence of SEQ ID NO: 230, 231, 232, or 233, (ii) a sequence comprising one or more mutations, optionally one, two, three, four, or five mutations, relative to SEQ ID NO: 230, 231, 232, or 233, optionally wherein the one or more mutations are at any nucleotide position(s) or are at position(s) other than the 4^{th} to the 7^{th} nucleotide positions from the 3′-end of SEQ ID NO: 230, 231, 232, or 233, or (iii) a sequence comprising at least 85, 90, 95, 96, 97, 98 or 99% sequence identity to SEQ ID NO: 230, 231, 232, or 233.

In some embodiments, the one or more RNPs may comprise all of: (I-1) the first isolated gRNA which comprises the first targeting sequence of SEQ ID NO: 210, 211, 212, or 213; (I-2) the second isolated gRNA comprising the second targeting sequence of SEQ ID NO: 220, 221, 222, or 223; and (I-3) the third isolated gRNA comprising the third targeting sequence of SEQ ID NO: 230, 231, 232, or 233, and optionally (I-4) one or more isolated gRNAs for editing exon 5 of HTT or its proximate gene segment described above.

In some embodiments, the one or more RNPs may comprise one or more of the following (II-1)-(II-3):

(II-1) a fourth isolated gRNA for editing exon 8 of HTT or its proximate gene segment described above, which may comprise a fourth targeting sequence comprising or consisting of: (i) a sequence of SEQ ID NO: 310, 311, 312, or 313, (ii) a sequence comprising one or more mutations, optionally one, two, three, four, or five mutations, relative to SEQ ID NO: 310, 311, 312, or 313, optionally wherein the one or more mutations are at any nucleotide position(s) or are at position(s) other than the 4^{th} to the 7^{th} nucleotide positions from the 3′-end of SEQ ID NO: 310, 311, 312, or 313, or (iii) a sequence comprising at least 85, 90, 95, 96, 97, 98 or 99% sequence identity to SEQ ID NO: 310, 311, 312, or 313;

(II-2) a fifth isolated gRNA for editing exon 8 of HTT or its proximate gene segment described above, which may comprise a fifth targeting sequence comprising or consisting of: (i) a sequence of SEQ ID NO: 320, 321, 322, or 323, (ii) a sequence comprising one or more mutations, optionally one, two, three, four, or five mutations, relative to SEQ ID NO: 320, 321, 322, or 323, optionally wherein the one or more mutations are at any nucleotide position(s) or are at position(s) other than the 4^{th} to the 7^{th} nucleotide positions from the 3′-end of SEQ ID NO: 320, 321, 322, or 323, or (iii) a sequence comprising at least 85, 90, 95, 96, 97, 98 or 99% sequence identity to SEQ ID NO: 320, 321, 322, or 323; and/or

(II-3) a sixth isolated gRNA for editing exon 8 of HTT or its proximate gene segment described above, which may comprise a sixth targeting sequence comprising or consisting of: (i) a sequence of SEQ ID NO: 330, 331, 332, or 333, (ii) a sequence comprising one or more mutations, optionally one, two, three, four, or five mutations, relative to SEQ ID NO: 330, 331, 332, or 333, optionally wherein the one or more mutations are at any nucleotide position(s) or are at position(s) other than the 4^{th} to the 7^{th} nucleotide positions from the 3′-end of SEQ ID NO: 330, 331, 332, or 333, or (iii) a sequence comprising at least 85, 90, 95, 96, 97, 98 or 99% sequence identity to SEQ ID NO: 330, 331, 332, or 333.

In some embodiments, the one or more RNPs may comprise all of: (II-1) the fourth isolated gRNA which comprises the fourth targeting sequence of SEQ ID NO: 310, 311, 312, or 2313; (II-2) the fifth isolated gRNA comprising the fifth targeting sequence of SEQ ID NO: 320, 321, 322, or 323; and (II-3) the sixth isolated gRNA comprising the sixth targeting sequence of SEQ ID NO: 330, 331, 332, or 333, and optionally (II-4) one or more isolated gRNAs for editing exon 8 of HTT or its proximate gene segment described above.

In some embodiments, the one or more RNPs may comprise one or more of the following (III-1)-(III-4):

(III-1) a seventh isolated gRNA for editing exon 31 of HTT or its proximate gene segment described above, which comprises a seventh targeting sequence comprising or consisting of: (i) a sequence of SEQ ID NO: 410, 411, 412, or 413, (ii) a sequence comprising one or more mutations, optionally one, two, three, four, or five mutations, relative to SEQ ID NO: 410, 411, 412, or 413, optionally wherein the one or more mutations are at any nucleotide position(s) or are at position(s) other than the 4^{th} to the 7^{th} nucleotide positions from the 3′-end of 410, 411, 412, or 413, or (iii) a sequence comprising at least 85, 90, 95, 96, 97, 98 or 99% sequence identity to SEQ ID NO: 410, 411, 412, or 413;

(III-2) an eighth isolated gRNA for editing exon 31 of HTT or its proximate gene segment described above, which comprises an eighth targeting sequence comprising or consisting of: (i) a sequence of SEQ ID NO: 420, 421, 422, or 423, (ii) a sequence comprising one or more mutations, optionally one, two, three, four, or five mutations, relative to SEQ ID NO: 420, 421, 422, or 423, optionally wherein the one or more mutations are at any nucleotide position(s) or are at position(s) other than the 4^{th} to the 7^{th} nucleotide positions from the 3′-end of SEQ ID NO: 420, 421, 422, or 423, or (iii) a sequence comprising at least 85, 90, 95, 96, 97, 98 or 99% sequence identity to SEQ ID NO: 420, 421, 422, or 423;

(III-3) a ninth, isolated gRNA for editing exon 31 of HTT or its proximate gene segment described above, which comprises a ninth, targeting sequence comprising or consisting of: (i) a sequence of SEQ ID NO: 430, 431, 432, or 433, (ii) a sequence comprising one or more mutations, optionally one, two, three, four, or five mutations, relative to SEQ ID NO: 430, 431, 432, or 433, optionally wherein the one or more mutations are at any nucleotide position(s) or are at position(s) other than the 4^{th} to the 7^{th} nucleotide positions from the 3′-end of SEQ ID NO: 430, 431, 432, or 433, or (iii) a sequence comprising at least 85, 90, 95, 96, 97, 98 or 99% sequence identity to SEQ ID NO: 430, 431, 432, or 433; and/or

(III-4) a tenth isolated gRNA for editing exon 31 of HTT or its proximate gene segment described above, which comprises a tenth targeting sequence comprising or consisting of: (i) a sequence of SEQ ID NO: 440, 441, 442, or 443 or 450, 451, 452, or 453, (ii) a sequence comprising one or more mutations, optionally one, two, three, four, or five mutations, relative to SEQ ID NO: 440, 441, 442, or 443 or 450, 451, 452, or 453, optionally wherein the one or more mutations are at any nucleotide position(s) or are at position(s) other than the 4^{th} to the 7^{th} nucleotide positions from the 3′-end of SEQ ID NO: 440, 441, 442, or 443 or 450, 451, 452, or 453, or (iii) a sequence comprising at least 85, 90, 95, 96, 97, 98 or 99% sequence identity to SEQ ID NO: 440, 441, 442, or 443 or 450, 451, 452, or 453.

In some embodiments, the one or more RNPs may comprise all of: (III-1) the seventh isolated gRNA which comprises the seventh targeting sequence of SEQ ID NO: 410, 411, 412, or 413, (III-2) the eighth isolated gRNA comprising the eighth targeting sequence of SEQ ID NO: 420, 421, 422, or 423, and (III-3) the ninth, isolated gRNA comprising the ninth, targeting sequence of SEQ ID NO: 430, 431, 432, or 433, and optionally (III-5) one or more isolated gRNAs for editing exon 31 of HTT or its proximate gene segment described above.

In some embodiments, the one or more RNPs may comprise all of: (III-1) the seventh isolated gRNA which comprises the seventh targeting sequence of SEQ ID NO: 410, 411, 412, or 413, (III-3) the ninth, isolated gRNA comprising the ninth, targeting sequence of SEQ ID NO: 430, 431, 432, or 433, and (III-4) the tenth isolated gRNA comprising the tenth targeting sequence of SEQ ID NO: 440, 441, 442, or 443 or 450, 451, 452, or 453, and optionally (III-5) one or more isolated gRNAs for editing exon 31 of HTT or its proximate gene segment described above.

In certain embodiments, the Cas endonuclease may be selected from the group consisting of Cas9, Cas3, Cas8a2, Cas8b, Cas8c, Cas10, Cas11, Cas12, Cas12a or Cpf1, Cas13, Cas13a, C2c1, C2c3, and C2c2. In certain embodiments, the Cas endonuclease may be a class 2 Cas endonuclease, optionally a type II, type V, or type VI Cas nuclease. In certain embodiments, the Cas endonuclease may be Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, eSpCas9, HypaCas9, FokI-Fused dCas9, xCas9, SpRY (variant of SpCas9), and SpG (variant of SpCas9). In certain embodiments, the Cas endonuclease may be Cas9, optionally comprising any one of SEQ ID NOS: 600-611.

In some embodiments, the pharmaceutically acceptable carrier may comprise a lipid-based TCV.

In some embodiments, the composition may comprise, in (B), (a) the one or more isolated gRNAs, and (b) the Cas endonuclease. In some embodiments, the composition may comprise, in (B), (a) the one or more isolated gRNAs, and (b) a vector comprising the polynucleotide encoding a Cas endonuclease. In some embodiments, the composition may comprise, in (B), (a) one or more vectors comprising the one or more polynucleotides encoding the one or more isolated gRNAs, and (b) the Cas endonuclease. In some embodiments, the composition may comprise, in (B), (a) one or more vectors comprising the one or more polynucleotides encoding the one or more isolated gRNAs, and (b) a vector comprising the polynucleotide encoding a Cas endonuclease.

In certain embodiments, the vector or vector(s) may be individually selected from plasmids, RNA replicons, virus-like particles (VLPs), and viral vectors, optionally retroviral, lentiviral, or adenoviral vectors.

In certain embodiments, when one or more isolated gRNAs are more than one isolated gRNAs, the more than one gRNAs may be encoded in a single vector or in separate vectors. In certain embodiments, the one or more isolated gRNAs and the Cas endonuclease may be encoded in a single vector or in separate vectors.

In any of the compositions described herein, the composition may, in some embodiments, comprise one or more template DNAs. Any appropriate template DNAs may be used, which may or may not be one or more of the template DNAs specifically described herein. Template DNAs may be selected based on the sites (e.g., sequences around the sites cleaved by a Cas nuclease) intended to join. In some embodiments, the template DNA may comprise single-strand oligo DNA nucleotide molecules (ssODNs), which may comprise or consist of a 5′ homology arm, an optional central region, and a 3′ homology arm. In some embodiments, the template DNA may be a double-strand DNA molecule.

In some embodiments, when the composition is for editing at least exon 5 of HTT or its proximate gene segment as described above, the ssODN that may be comprised may comprise or consist of: (i) the 5′ homology arm comprises or consists of (i-1) the sequence corresponding to the first nucleotide to at least the 10^{th} nucleotide counting from the 3′-end of SEQ ID NO: 268, (i-2) the sequence of any of SEQ ID NOS: 261-268, or (i-3) a sequence comprising at least one mutation, optionally one, two, three, four, five, six, seven, eight, nine, or ten mutation(s), relative to the sequence of (i-1) or (i-2), (ii) the optional central region is 1-100 nucleotides (nt) in length; and (iii) the 3′ homology arm comprises or consists of (iii-1) the sequence corresponding to the first nucleotide to at least the 10^{th} nucleotide counting from the 5′-end of SEQ ID NO: 278, (iii-2) the sequence of any of SEQ ID NOS: 271-278, or (iii-3) a sequence comprising at least one mutation, optionally one, two, three, four, five, six, seven, eight, nine, or ten mutation(s), relative to the sequence of (iii-1) or (iii-2).

In certain embodiments, the ssODN may comprise or consist of the sequence of any of SEQ ID NOs: 281-288.

In some embodiments, the sequence of the ssODN may be fully complementary to the sequence any of the ssODNs described above for a composition for editing at least exon 5 of HTT or its proximate gene segment. In certain embodiments, the ssODN may comprise or consist of the sequence of any of SEQ ID NOs: 291-298.

In some embodiments, a double-strand DNA molecule may comprise: a first strand comprising any of the ssODN sequences described above (for exon 5 and its proximate gene segment) and a second strand complementary to the first strand.

In some embodiments, when the composition is for editing at least exon 8 of HTT or its proximate gene segment as described above, the ssODN that may be comprised may comprise or consist of: (i) the 5′ homology arm comprises or consists of (i-1) the sequence corresponding to the first nucleotide to at least the 10^{th} nucleotide counting from the 3′-end of SEQ ID NO: 368, (i-2) the sequence of any of SEQ ID NOS: 361-368, or (i-3) a sequence comprising at least one mutation, optionally one, two, three, four, five, six, seven, eight, nine, or ten mutation(s), relative to the sequence of (i-1) or (i-2), (ii) the optional central region is 1-100 nucleotides (nt) in length; and (iii) the 3′ homology arm comprises or consists of (iii-1) the sequence corresponding to the first nucleotide to at least the 10^{th} nucleotide counting from the 5′-end of SEQ ID NO: 378, (iii-2) the sequence of any of SEQ ID NOS: 371-378, or (iii-3) a sequence comprising at least one mutation, optionally one, two, three, four, five, six, seven, eight, nine, or ten mutation(s), relative to the sequence of (iii-1) or (iii-2).

In certain embodiments, the ssODN may comprise or consist of the sequence of any of SEQ ID NOs: 381-388.

In some embodiments, the sequence of the ssODN may be fully complementary to the sequence any of the ssODNs described above for a composition for editing at least exon 8 of HTT or its proximate gene segment. In certain embodiments, the ssODN may comprise or consist of the sequence of any of SEQ ID NOs: 391-398.

In some embodiments, a double-strand DNA molecule may comprise: a first strand comprising any of the ssODN sequences described above (for exon 8 and its proximate gene segment) and a second strand complementary to the first strand.

In some embodiments, when the composition is for editing at least exon 31 of HTT or its proximate gene segment as described above, the ssODN that may be comprised may comprise or consist of: (i) the 5′ homology arm comprises or consists of (i-1) the sequence corresponding to the first nucleotide to at least the 10^{th} nucleotide counting from the 3′-end of SEQ ID NO: 468, (i-2) the sequence of any of SEQ ID NOS: 461-468, or (i-3) a sequence comprising at least one mutation, optionally one, two, three, four, five, six, seven, eight, nine, or ten mutation(s), relative to the sequence of (i-1) or (i-2), (ii) the optional central region is 1-100 nucleotides (nt) in length; and (iii) the 3′ homology arm comprises or consists of (iii-1) the sequence corresponding to the first nucleotide to at least the 10^{th} nucleotide counting from the 5′-end of SEQ ID NO: 478, (iii-2) the sequence of any of SEQ ID NOS: 471-478, or (iii-3) a sequence comprising at least one mutation, optionally one, two, three, four, five, six, seven, eight, nine, or ten mutation(s), relative to the sequence of (iii-1) or (iii-2).

In certain embodiments, the ssODN may comprise or consist of the sequence of any of SEQ ID NOs: 481-488.

In some embodiments, the sequence of the ssODN may be fully complementary to the sequence any of the ssODNs described above for a composition for editing at least exon 31 of HTT or its proximate gene segment. In certain embodiments, the ssODN may comprise or consist of the sequence of any of SEQ ID NOs: 491-498.

In some embodiments, a double-strand DNA molecule may comprise: a first strand comprising any of the ssODN sequences described above (for exon 31 and its proximate gene segment) and a second strand complementary to the first strand.

Any of the compositions described herein may comprise a pharmaceutically acceptable carrier, and in some embodiments a TCV, such as a lipid-based TCV.

In some embodiments, the TCV may comprise at least one cationic or ionizable cationic lipid.

In certain embodiments, the least one cationic or ionizable cationic lipid may comprise, essentially consist of, or consist of a lipid selected from the group consisting of DODMA, DODAP, DLinDAP, KC2, MC3, DODAC, DDAB, DOTAP, DOTMA, DLinDMA, DLenDMA, DLin-C-DAP, DLin-DAC, DLin-MA, DLin-S-DMA, DLin-2-DMAP, DLin-TMA.Cl, DLin-TAR.Cl, DLin-MPZ, or DLinAP, DOAP, DLin-EG-DMA, DLin-K-DMA, DLin-K-DMA or analogs thereof, ALNY-100, DOTMA; 1,2-DOTAP.Cl; DC-Chol, DOSPA, DOGS, and DMRIE, and any combinations thereof.

In certain embodiments, the TCV may further comprise at least one helper lipid, optionally wherein the at least one helper lipid may comprise, essentially consist of, or consist of a lipid selected from the group consisting of DOPE, DSPC, DOPC, DPPC, DOPG, DPPG, POPC, POPE, DOPE-mal, DPPE, DMPE, DSPE, 16-O-monomethyl PE, 16-O-dimethyl PE, 18-1-trans PE, SOPE, and any combinations thereof.

In certain embodiments, the TCV may further comprise at least one phospholipid, optionally wherein the at least one phospholipid may comprise, essentially consist of, or consist of a lipid selected from the group consisting of DSPC, DOPE, DPPC, DOPC, DMPC, PLPC, DAPC, PE, EPC, DLPC, DMPC, MPPC, PMPC, PSPC, DBPC, SPPC, DEPC, POPC, lysophosphatidyl choline, dilinoleoylphosphatidylcholine, DSPE, DMPE, DPPE, POPE, lysophosphatidylethanolamine, and any combinations thereof.

In certain embodiments, the TCV may further comprise at least one cholesterol or cholesterol derivative, optionally wherein the at least one cholesterol or cholesterol derivative comprises, essentially consists of, or consists of a cholesterol or cholesterol derivative selected from the group consisting of cholesterol, DC-Chol, 1,4-bis(3-N-oleylamino-propyl)piperazine, ICE, and any combinations thereof.

In certain embodiments, the TCV may further comprise at least one PEG or PEG-lipid, optionally wherein the at least one PEG-lipid comprises, essentially consists of, or consists of a PEG-lipid selected from the group consisting of PEG-DMG (e.g., 1,2-dimyristoyl-rac-glycero-3-methoxypolyethylene glycol-2000 (Avanti® Polar Lipids (Birmingham, AL)), which is a mixture of 1,2-DMG PEG2000 and 1,3-DMG PEG2000 (e.g., in about 97:3 ratio)), PEG-phosphatidylethanolamine and phosphatidic acid, PEG-ceramide conjugates (e.g., PEG-CerC14 or PEG-CerC20), PEG-modified dialkylamines, PEG-modified 1,2-diacyloxypropan-3-amines, and any combination thereof.

In particular embodiments, the TCV may be substantially, essentially, or entirely free of any permanently cationic lipids and/or any permanently anionic lipids.

In certain embodiments, the TCV may be: substantially, essentially, or entirely free of ethanol, methanol, isopropanol, THF, DMSO, DMF, and ACN; and/or substantially, essentially, or entirely free of SDS; optionally substantially, essentially, or entirely free of organic solvents and/or detergents; further optionally substantially, essentially, or entirely free of any other destabilizing agents.

In certain embodiments, the TCV may be formed by: (a) providing a first solution comprising all components of the TCV, optionally at about 20-35 mM, in ethanol; (b) providing a second solution, which is aqueous and contains acetate and/or citrate, optionally sodium acetate and/or sodium citrate, optionally at about 25 mM, optionally wherein the pH of the solution is about 3.5 to 4.5 or about 4; (c) combining the first and second solutions by gentle mixing (optionally repeated manual reciprocation of the TCV-generating fluid in a pipette), mixing using a staggered herringbone micromixer (SHM), T-junction mixing, or extrusion; and (d) removing ethanol, optionally by dialysis or evaporation.

In certain embodiments, the size of the TCV before encapsulation may be in a range of about 9 nm to about 80 nm, optionally about 10-40 nm, further optionally about 20-35 nm, at pH of about 3.5 to 4.5 or about 4.

In certain embodiments, the size of the TCV after encapsulation of the at least one cargo may be in a range of about 80 nm to about 1500 nm, optionally about 800 nm to about 1400 or about 1000 nm to about 1200 nm or about 80 nm to about 300 nm or about 100 nm to about 250 nm.

In certain embodiments, the TCV after encapsulation may be further comprised in a matrix vesicle, which is optionally for gradual release of the TCV.

In certain embodiments, the final ethanol concentration of the composition may be 5% (v/v) or below, 2.5% (v/v) or below, 1.5% (v/v) or below, and preferably 0.5% (v/v) or below.

In some cases, the amount of the at least one cationic or ionizable cationic lipid relative to the total components of the TCV may be: (a-1) about 10 mol % to about 70 mol %, about 10 mol % to about 60 mol %, about 10 mol % to about 50 mol %, about 10 mol % to about 40 mol %, about 10 mol % to about 30 mol %, about 15 mol % to about 25 mol %, about 18 mol % to about 22 mol %, about 19 mol % to about 21 mol %, about 19.5 mol % to about 20.5 mol %, about 19.8 mol % to about 20.2 mol %, or about 20 mol %; or (a-2) about 10 mol % to about 70 mol %, about 20 mol % to about 70 mol %, about 30 mol % to about 70 mol %, about 40 mol % to about 70 mol %, about 40 mol % to about 60 mol %, about 45 mol % to about 55 mol %, about 48 mol % to about 52 mol %, about 49 mol % to about 51 mol %, about 49.5 mol % to about 50.5 mol %, about 49.8 mol % to about 50.2 mol %, or about 50 mol %.

In some cases, the amount of the at least one helper lipid relative to the total components of the TCV may be about 10 mol % to about 60 mol %, about 10 mol % to about 50 mol %, about 10 mol % to about 40 mol %, about 20 mol % to about 40 mol %, about 25 mol % to about 35 mol %, about 28 mol % to about 32 mol %, about 29 mol % to about 31 mol %, about 29.5 mol % to about 30.5 mol %, about 29.8 mol % to about 30.2 mol %, or about 30 mol %.

In some cases, the amount of the at least one phospholipid relative to the total components of the TCV is about 5 mol % to about 65 mol %, about 5 mol % to about 55 mol %, about 5 mol % to about 45 mol %, about 5 mol % to about 35 mol %, about 5 mol % to about 25 mol %, about 5 mol % to about 15 mol %, about 8 mol % to about 12 mol %, about 9 mol % to about 11 mol %, about 9.5 mol % to about 10.5 mol %, about 9.8 mol % to about 10.2 mol %, or about 10 mol %.

In some cases, the amount of the at least one cholesterol or cholesterol derivative relative to the total components of the TCV may be about 20 mol % to about 60 mol %, about 25 mol % to about 55 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %, or about 39%.

In some cases, the amount of the at least one PEG or PEG-lipid relative to the total components of the TCV is about 0.1 mol % to about 5 mol %, 0.1 mol % to about 4 mol %, 0.1 mol % to about 3 mol %, 0.1 mol % to about 2 mol %, 0.5 mol % to about 1.5 mol %, 0.8 mol % to about 1.2 mol %, 0.9 mol % to about 1.1 mol %, or about 1 mol %.

In particular embodiments, the TCV may comprise, essentially consist of, or consist of: (i) at least one ionizable cationic lipid, which is optionally DODMA; (ii) at least one helper lipid, which is optionally DOPE; (iii) at least one phospholipid, which is optionally DSPC; and (iv) at least one cholesterol or cholesterol derivative.

In some cases, the amounts of the at least one ionizable cationic lipid, the at least one helper lipid, the at least one phospholipid, and the at least one cholesterol or cholesterol derivative, relative to the total components of the TCV, may be about 20 mol %, about 30 mol %, about 10 mol %, and about 40 mol %, respectively.

In particular embodiments, the TCV may comprise, essentially consist of, or consist of: (i) at least one ionizable cationic lipid, which is optionally DODMA; (ii) at least one helper lipid, which is optionally DOPE; (iii) at least one phospholipid, which is optionally DSPC; (iv) at least one cholesterol or cholesterol derivative; and (v) at least one PEG or PEG-lipid, which is optionally PEG-DMG.

In some cases, the amounts of the at least one ionizable cationic lipid, the at least one helper lipid, the at least one phospholipid, the at least one cholesterol or cholesterol derivative, and the at least one PEG or PEG-lipid, relative to the total components of the TCV, may be about 20 mol %, about 30 mol %, about 10 mol %, about 39 mol %, and about 1 mol %, respectively.

In certain embodiments, the TCV may be stable for prolonged periods of time at about 1 to about 40° C., about 5 to about 35° C., about 10 to about 30° C., or about to about 25° C.

In certain embodiments, the TCV or the composition may further comprise and/or may be stored in the presence of at least one cryoprotectant. In some cases, the cryoprotectant may comprise a sugar-based molecule, which is optionally sucrose, trehalose, or a combination thereof. In some cases, the concentration of the cryoprotectant may be about 1% to about 40%, about 3% to about 30%, about 5% to about 30%, about 10% to about 20%, or about 15%. In some cases, the TCV may be stable at a freezing temperature, optionally at about −20° C. or about −80° C., optionally for at least about one week, at least about two weeks, at least about three weeks, at least about a month, at least about two months, at least about four months, at least about five months, at least about six months, at least about nine months, at least about a year, or at least about two years, or longer, further optionally for about one week to about two year, about two weeks to about a year, about three weeks to about nine month, about one to about six months, about one to five months, about one to four months, about one to three months, or about one to two months.

Any of the compositions described herein may, in some embodiments, further comprise one or more agents for preventing, ameliorating, slowing the progression of, and/or treating HD.

In some embodiments, the one or more agents may comprise a drug for controlling movements, optionally tetrabenazine, deutetrabenazine, haloperidol, fluphenazine, risperidone, olanzapine, quetiapine, amantadine, levetiracetam, and/or clonazepam. In some embodiments, the one or more agents may comprise a drug for controlling psychiatric disorders, optionally (i) an antidepressant, optionally citalopram, escitalopram, fluoxetine, and/or sertraline, (ii) an antipsychotic drug, optionally quetiapine, risperidone, and olanzapine, and/or (iii) a mood-stabilizing drug, optionally divalproex, carbamazepine, and/or lamotrigine.

Some aspects of the present disclosure relate to methods for effecting CRISPR-mediated gene editing of the huntingtin gene and/or for preventing, ameliorating, slowing the progression of, and/or treating HD and/or one or more symptoms of HD in a subject.

In one aspect, the present disclosure provides methods for effecting CRISPR-mediated gene editing of the huntingtin gene, such as the human huntingtin gene, HTT, in one or more target cells and/or one or more target tissues.

In some embodiments, a method may comprise applying any of the compositions described herein to the one or more cells or one or more tissues.

In certain embodiments, the one or more target cells may comprise one or more of the following: (i) a mammalian cell, a human cell, a cell line, a stem cell-derived cell, an iPSC-derived cell, a primary cell, and/or a cell derived from a subject who has or has a risk of developing HD; (ii) a brain cell, optionally a neural cell, a cortical neuron, a cell of the basal ganglia, a cell of the striatum, a cell of the caudate nucleus, and/or a cell of the putamen, (iii) a myocyte, optionally a skeletal myocyte, a cardiomyocyte, and/or a smooth myocyte; (iv) a cell of an endocrinal system, optionally a pancreatic cell and/or an adipocyte; (v) a blood cell, optionally a lymphocyte, a macrophage, and/or a monocyte; and/or (vi) a fibroblast and/or a lymphoblast.

In certain embodiments, the one or more target tissues may comprise one or more of the following: (vii) a mammalian tissue, a human tissue, a primary tissue, and/or a tissue from a subject who has or has a risk of developing HD; (viii) a brain tissue, optionally a nervous tissue, a tissue of the basal ganglia, a tissue of the striatum, a tissue of the caudate nucleus, and/or a tissue of the putamen; (ix) a muscle tissue, optionally a skeletal muscle tissue, a cardiac tissue, and/or a smooth muscle tissue; (x) an endocrine tissue, optionally a pancreatic tissue and/or an adipose tissue; and/or (xi) a lymphoid and/or myeloid tissue, optionally a tissue of the bone marrow, a thymic tissue, a lymph node tissue, a spleen tissue, a tissue of the tonsil, and/or a tissue of the Peyer's patches.

In certain embodiments, the method may further comprise analyzing the level of CRISPR-mediated gene editing events in said one or more target cells or said one or more target tissues.

In one aspect, the present disclosure provides methods for effecting CRISPR-mediated gene editing of the huntingtin gene, such as the human huntingtin gene, HTT, in one or more target cells and/or one or more target tissues in vivo in a subject in need thereof.

In some embodiments, the method may comprise administering any of the compositions described herein to the subject, e.g., by intraspinal or intrathecal administration, optionally by use of a pump and/or catheter or by surgical means or other suitable administration means.

In certain embodiments, the one or more target cells may comprise one or more of the following: (i) a mammalian cell, a human cell, a cell line, a stem cell-derived cell, an iPSC-derived cell, a primary cell, and/or a cell derived from a subject who has or has a risk of developing HD; (ii) a brain cell, optionally a neural cell, a cortical neuron, a cell of the basal ganglia, a cell of the striatum, a cell of the caudate nucleus, and/or a cell of the putamen, (iii) a myocyte, optionally a skeletal myocyte, a cardiomyocyte, and/or a smooth myocyte; (iv) a cell of an endocrinal system, optionally a pancreatic cell and/or an adipocyte; (v) a blood cell, optionally a lymphocyte, a macrophage, and/or a monocyte; and/or (vi) a fibroblast and/or a lymphoblast.

In certain embodiments, the one or more target tissues may comprise one or more of the following: (vii) a mammalian tissue, a human tissue, a primary tissue, and/or a tissue from a subject who has or has a risk of developing HD; (viii) a brain tissue, optionally a nervous tissue, a tissue of the basal ganglia, a tissue of the striatum, a tissue of the caudate nucleus, and/or a tissue of the putamen; (ix) a muscle tissue, optionally a skeletal muscle tissue, a cardiac tissue, and/or a smooth muscle tissue; (x) an endocrine tissue, optionally a pancreatic tissue and/or an adipose tissue; and/or (xi) a lymphoid and/or myeloid tissue, optionally a tissue of the bone marrow, a thymic tissue, a lymph node tissue, a spleen tissue, a tissue of the tonsil, and/or a tissue of the Peyer's patches.

In certain embodiments, the administrating may be effected to reach or until reaching a minimum of about 10%, about 15%, about 20%, about 30%, or an about final 15-30% or about final 20-40% target cells and/or tissue(s) with successful gene editing among the total target cells and/or tissue(s). In some cases, the administration dose and/or frequencies may be adjusted to achieve such gene editing levels.

In certain embodiments, the method is for preventing, ameliorating, slowing the progression of, and/or treating HD and/or one or more symptoms of HD in the subject.

In certain embodiments, the effect of the method may be evaluated based on any one or more of the following: (I) the number of the target cells with successful gene editing; (II) the % of the target cells with successful gene editing among total target cells; (III) the % of the target tissue(s) with successful gene editing out of total target tissue(s); (IV) the expression level of the HTT gene in the one or more target cells; (V) the expression level of the HTT gene in the one or more target tissues. In certain embodiments, the effect of the method may be evaluated based on changes in any one or more of the following: (a) the level of aggregation of huntingtin protein, optionally in the one or more target cells, further optionally in neural cells, brain cells, and/or muscle cells, (b) the level of aggregation of huntingtin protein in one or more target tissues, optionally in the brain, (c) the level of cell death, optionally of the one or more target cells, further optionally of neural cells, brain cells, and/or muscle cells, optionally via apoptosis or autophagy, (d) the level of cell death in the one or more target tissues, optionally in the brain, the basal ganglia, the striatum, the caudate nucleus, and/or the putamen, optionally via apoptosis or autophagy, (e) the level of inflammation, optionally the level of one or more cytokines (optionally IL-4, IL-6, and/or TNF-alpha), optionally produced or released by the one or more target cells, further optionally neural cells, brain cells, immune cells, macrophages, and/or monocytes, (f) the level of inflammation, optionally the level of one or more cytokines (optionally IL-4, IL-6, and/or TNF-alpha), in the one or more target tissues, optionally the brain, the basal ganglia, the striatum, the caudate nucleus, and/or the putamen, (g) mitochondrial function, optionally in the one or more target cells, further optionally in lymphocytes and/or myocytes, further optionally cardiomyocytes and/or skeletal myocytes, (h) activity or activation of caspase, optionally caspase 3 and/or caspase 9, optionally in the one or more target cells, further optionally in myocytes, (i) the level of insulin expression and/or release by pancreatic cells, and/or (j) the level of leptin release by adipose tissue.

In one aspect, the present disclosure provides methods of preventing, ameliorating, slowing the progression of, and/or treating HD and/or one or more symptoms of HD in a subject in need thereof.

In some embodiments, the method may comprise administering any of the compositions described herein to the subject, optionally according to the in vivo method described above.

In certain embodiments, the method may further comprise administering one or more agents for preventing, ameliorating, slowing the progression of, or treating HD, which optionally comprise(s): (A) a drug for controlling movements, optionally tetrabenazine, deutetrabenazine, haloperidol, fluphenazine, risperidone, olanzapine, quetiapine, amantadine, levetiracetam, and/or clonazepam; and/or (B) a drug for controlling psychiatric disorders, optionally (a) an antidepressant, optionally citalopram, escitalopram, fluoxetine, and/or sertraline, (b) an antipsychotic drug, optionally quetiapine, risperidone, and olanzapine, and/or (c) a mood-stabilizing drug, optionally divalproex, carbamazepine, and/or lamotrigine.

In some cases, the one or more agents may be administered (i) separately from the composition or (ii) together with the composition.

In some embodiments, the one or more symptoms of HD may be any HD symptoms, such as but not limited to any one or more of the following: (a) cognitive deficits, optionally (i) cognitive slowing, (ii) decreases in attention, and/or (iii) decreases in mental flexibility; (b) psychiatric disruption and/or emotional deficits, optionally (i) depressed, (ii) apathy, (iii) irritability, (iv) impulsivity, and/or (v) social disinhibition; (c) loss of motor coordination, optionally (i) choreiform movements, (ii) gait disturbances, and/or (ii) bradykinesia, and/or (iv) rigidity; (d) weight loss; and/or (e) inflammation, optionally neural inflammation or inflammation in the brain.

In some embodiments, the effect of the method may be evaluated based on the changes in (the) one or more symptoms of HD.

In any of the in vivo methods described herein, the subject may be a mammal, optionally: (i) a human subject, optionally a human subject who has or has a risk of developing HD; or (ii) a non-human subject, optionally a non-human primate or selected from a rodent (mouse, rat, guinea pig, hamster), rabbit, cat, dog, pig, goat, sheep, horse, or monkey, further optionally a mouse or a rat.

In any of the in vivo methods described herein, the composition is administered parenterally, optionally: (i) by injection, further optionally: direct injection into the central nervous system (CNS); intracranial injection; direct injection into the brain; direct injection into the basal ganglia; direct injection into the striatum; direct injection into the caudate nucleus and/or putamen; intraspinal (IS) injection, intrathecal (IT) injection; intravenous (IV) injection; subcutaneous (SC) injection; intramuscular (IM) injection; intradermal (ID) injection; intra-arterial (IA) injection; intraperitoneal (IP) injection; or intravitreal injection, (ii) by inhalation or via a pulmonary route, or (iii) transdermally.

In any of the in vivo methods described herein, the composition is administered locally, optionally topically to the skin or mucous membrane.

In any of the in vivo methods described herein, the composition is administered enterally, optionally orally, sublingually/buccally, or rectally.

In any of the in vivo methods described herein, the amount of the gRNA(s) or the gRNA-encoding polynucleotide(s) comprised in the composition for administration per mL may be about 300 to 30000 nmol, optionally about 500 to 10000 nmol, about 1000 to 5000 nmol, about 2000 to 4000 nmol, about 2500 to 3000 nmol, or about 2700 nmol.

In any of the in vivo methods described herein, the total volume comprising the composition for administration may be about 0.1-10000 μL, about 1-5000 μL, about 2-2000 μL, about 4-1000 μL, about 10-500 μL, or about 20-200 μL.

In any of the in vivo methods described herein, the administering comprises injecting the composition in a continuous flow of about 0.1 μL to 2000 μL per minute, optionally about 1 μL to 1000 μL per minute, about 2 μL to 500 μL per minute, or about 5 μL to about 100 μL per minute into the brain of the subject.

In any of the in vivo methods described herein, the administering may be effected twice or more, optionally about 3-5 times, optionally at different time points, further optionally with an interval of about a week, about two weeks, or about three weeks, about a month, about three months, about six months, about a year, about three years, or about six years.

In any of the in vivo methods described herein, the administering comprises at least two simultaneous or essentially simultaneous administrations at multiple sites of the subject, optionally in each hemisphere of the brain, further optionally in each striatum.

In any of the in vivo methods described herein, the subject may be about 20 years old or older, about 25 years old or older, about 30 years old or older, about 35 years old or older, about 40 years old or older or may be juvenile.

In any of the in vivo methods described herein, the subject may not have fully developed HD and/or may be treated prior to manifesting a symptom or complication or (b) may be treated at initial onset of HD.

Any of the compositions described herein may, in some embodiments, be contained in a kit, which may further comprise an instruction and/or a label. In certain embodiments, the instruction and/or label may be for use of the composition, which may be according to any of the methods described herein.

Any of the compositions described herein, in some embodiments, may be for use as a medicament. In certain embodiments, the use may be for preventing, ameliorating, slowing the progression of, and/or treating HD and/or for preventing, ameliorating, slowing the progression of, and/or treating one or more symptoms of HD. In certain embodiments, the use may be according to any of the methods described herein.

Any of the compositions described herein, in some embodiments, may be for the manufacture of a medicament, which may be for preventing, ameliorating, slowing the progression of, and/or treating HD or for preventing, ameliorating, slowing the progression of, and/or treating one or more symptoms of HD. In certain embodiments, such a medicament may be for use according to any of the methods described herein.

These and other aspects, features and embodiments are set forth within this application, including the following Detailed Description and attached drawings. Unless expressly stated otherwise, all embodiments, aspects, features, etc., can be mixed and matched, combined and permuted in any desired manner. In addition, various references are set forth herein, including in the Cross-Reference. To Related Applications, that discuss certain systems, apparatus, methods and other information; all such references are incorporated herein by reference in their entirety and for all their teachings and disclosures, regardless of where the references may appear in this application.

### BRIEF DESCRIPTION OF THE DRAWINGS

FIG. 1 depicts schematic diagrams of RNP-TCV preparation using multiple guide RNAs and exemplary gene targeting strategies.

FIG. 2A depicts an exemplary result from Example 1, demonstrating successful reduction in the HTT mRNA transcript in HEK cells by delivery of RNP-TCV targeting HTT exon 31 but not exon 2.

FIG. 2B depicts another exemplary result from Example 1, demonstrating dose-dependent reduction in the HTT transcript in HEK cells by delivery of RNP-TCV targeting HTT exon 31 either using a single gRNA or a mixture of three gRNAs.

FIG. 3A depicts an exemplary result from Example 2, demonstrating successful reduction in the mouse Htt transcript in primary neurons by RNP-TCV targeting human HTT exon 31.

FIG. 3B depicts another exemplary result from Example 2, demonstrating successful reduction in human HTT transcript in primary neurons by RNP-TCV targeting human HTT exon 31.

FIG. 4 depicts an exemplary result from Example 3, demonstrating successful reduction in human HTT transcript in human iPSC-derived neurons by RNP-TCV targeting human HTT exon 31.

FIG. 5 depicts an exemplary result from Example 4, a graph demonstrating successful reduction in human HTT transcript in HEK cells by delivery of RNP-TCV targeting human HTT exon 5, 8, or 31 or all of exons 5, 8, and 31.

FIG. 6A depicts exemplary body weight changes in mice in Example 5, demonstrating no notable effects on body weight by direct administration of RNP-TCV into the mouse brain.

FIG. 6B depicts an exemplary result from Example 5, demonstrating successful reduction in Htt transcript following delivery of RNP-TCV by direct injection into the mouse brain, regardless of whether the injection was given at a single site or two different sites.

FIG. 7A provides the gene sequence (SEQ ID NO: 201) around exon 5 of HTT. The sequence of exon 5 (SEQ ID NO: 200) is shown in bold. The regions corresponding to the targeting sequences of the three gRNAs specific to HTT exon 5 used in Examples are highlighted (SEQ ID NOS: 210 (or 4), 220 (or 5), and 230 (or 6)). The squares correspond to individual PAM sites for the gRNAs. The sequence of an exemplary template DNA that may be used in combination with such gRNAs is shown with an underline (SEQ ID NO: 284).

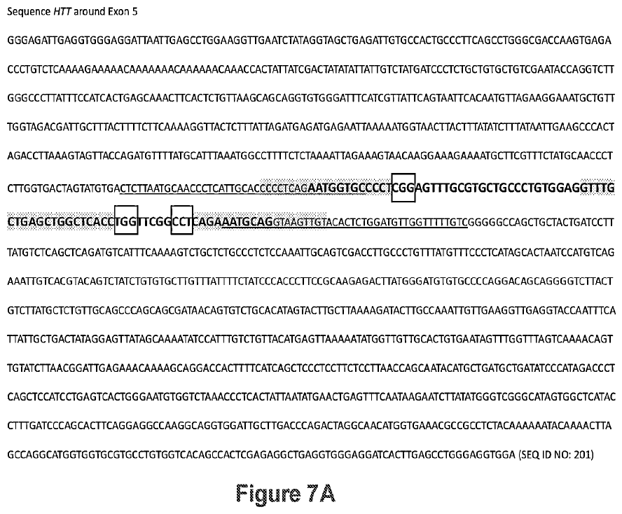

FIG. 7B provides the gene sequence (SEQ ID NO: 301) around exon 8 of HTT. The sequence of exon 8 (SEQ ID NO: 300) is shown in bold. The regions corresponding to the targeting sequences of the three gRNAs specific to HTT exon 8 used in Examples are highlighted (SEQ ID NOS: 310 (or 7), 320 (or 8), and 330 (or 9)). The squares correspond to individual PAM sites for the gRNAs. The sequence of an exemplary template DNA that may be used in combination with such gRNAs is shown with an underline (SEQ ID NO: 384).

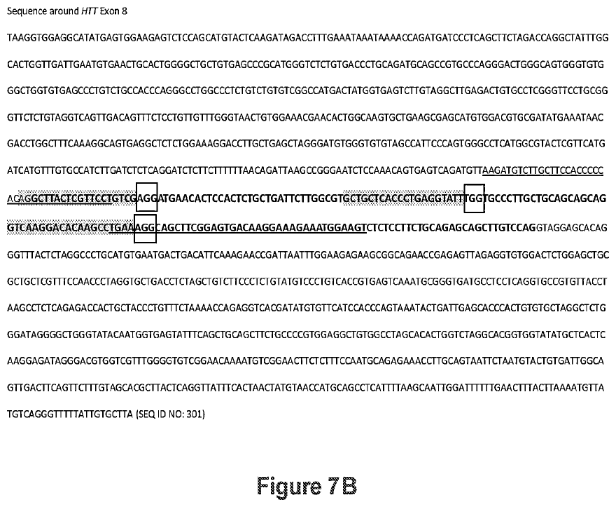

FIG. 7C provides the gene sequence (SEQ ID NO: 401) around exon 31 of HTT. The sequence of exon 31 (SEQ ID NO: 400) is shown in bold. The regions corresponding to the targeting sequences of the three gRNAs specific to HTT exon 31 used in Examples are highlighted (SEQ ID NOS: 410 (or 1), 420 (or 2), and 430 (or 3)). The squares correspond to individual PAM sites for the gRNAs. The sequence of an exemplary template DNA that may be used in combination with such gRNAs is shown with an underline (SEQ ID NO: 484).

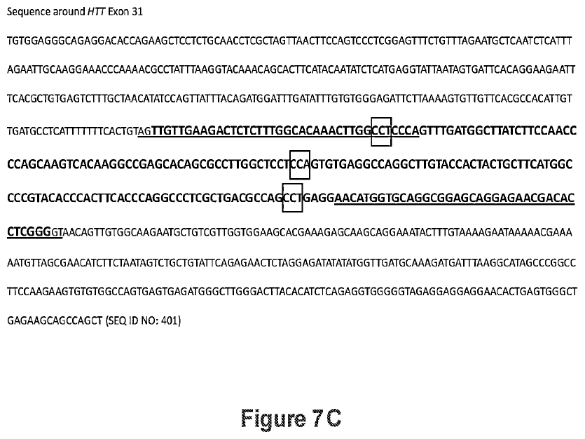

FIG. 7D provides the gene sequence (SEQ ID NO: 501) around exon 31 of Htt. The regions corresponding to the targeting sequences (SEQ ID NOS: 19, 20, and 21) of the three gRNAs specific to Htt exon 31 used in Examples are highlighted. The squares correspond to individual PAM sites for the gRNAs. The nucleotides within the targeting sequence-corresponding regions that are different from the corresponding human sequence are underlined.

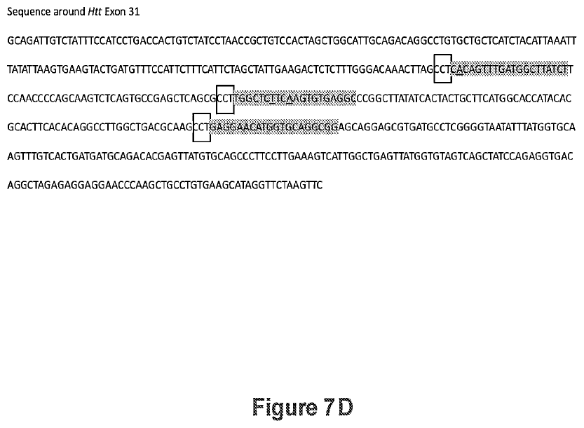

### DETAILED DESCRIPTION

The present disclosure provides, among other things, gRNAs, RNPs, compositions, and methods, which may be for effecting gene editing of the huntingtin gene and/or for preventing, ameliorating, slowing the progression of, and/or treating HD.

### Targets

### Target Diseases and Symptoms

In one aspect, a target disease according to the present disclosure may comprise a disease may be caused by the huntingtin gene, such as human huntingtin gene (HTT). In some embodiments, the disease may be caused by a mutant form of the huntingtin gene. In certain embodiments, the disease may be caused by a mutant huntingtin gene comprising expanded CAG repeats, for example in exon 1. In some embodiments, the disease may comprise HD. In certain embodiments, the disease may be HD which may be before its onset. In certain embodiments, the disease may be HD which may at its onset. In certain embodiments, the disease may be HD which may be in its early stage. In certain embodiments, the disease may be HD which may be in its mid stage. In certain embodiments, the disease may be HD which may be fully developed.

In one aspect, a target symptom according to the present disclosure may comprise a target caused by the huntingtin gene, such as human huntingtin gene (HTT). In some embodiments, the target symptom may be caused by a mutant form of the huntingtin gene. In certain embodiments, the target symptom may be caused by a mutant huntingtin gene comprising expanded CAG repeats, for example in exon 1, which confer a toxic gain-of-function to the gene product. In some embodiments, a target symptom may comprise a HD symptom.

In some embodiments, a target symptom or target symptoms may comprise cognitive deficit. In certain embodiments, a target symptom or target symptoms may comprise (i) cognitive slowing; (ii) decreases in attention, and/or (iii) decreases in mental flexibility. In some embodiments, a target symptom or target symptoms may comprise psychiatric disruption and/or emotional deficit. In certain embodiments, a target symptom or target symptoms may comprise (i) depression, (ii) apathy, (iii) irritability, (iv) impulsivity, and/or (v) social disinhibition. In some embodiments, a target symptom or target symptoms may comprise loss of motor coordination. In certain embodiments, a target symptom or target symptoms may comprise (i) choreiform movements, (ii) gait disturbances, and/or (ii) bradykinesia, and/or (iv) rigidity; (d) weight loss; and/or (e) inflammation, optionally neural inflammation or inflammation in the brain.

### Target Cells and Tissues

In one aspect, a target cell or target cells according to the present disclosure may comprise a cell or cells affected, to be affected, or likely affected by HD or associated with, involved in, and/or responsible for at least one of HD pathology and/or symptoms.

In some embodiments, the target cell or target cells may comprise a cell or cells of the brain. In certain embodiments, the target cell or target cells may comprise a neural cell, a cortical neuron, a cell of the basal ganglia, a cell of the striatum, a cell of the caudate nucleus, and/or a cell of the putamen. In some embodiments, the target cell or target cells may comprise a cell of the muscle. In certain embodiments, the target cell or target cells may comprise a myocyte, a skeletal myocyte, a cardiomyocyte, and/or a smooth myocyte. In some embodiments, the target cell or target cells may comprise a cell of an endocrinal system. In certain embodiments, the target cell or target cells may comprise a pancreatic cell and/or an adipocyte. In some embodiments, the target cell or target cells may comprise a cell or cells of the immune system. In certain embodiments, the target cell or target cells may comprise a blood cell, a lymphocyte, a macrophage, and/or a monocyte. In some embodiments, the target cell or target cells may comprise a fibroblast and/or a lymphoblast. In particular embodiments, the target cell or target cells may comprise a cell of the striatum.

In one aspect, a target tissue or target tissues according to the present disclosure may comprise a tissue or tissues affected, to be affected, or likely affected by HD or associated with, involved in, and/or responsible for at least one of HD pathology and/or symptoms. In some embodiments, the target tissue or tissues may comprise a CNS tissue. In certain embodiments, the target tissue or tissues may comprise a brain tissue. In particular embodiments, the target tissue or tissues may comprise a nervous tissue, a tissue of the basal ganglia, a tissue of the striatum, a tissue of the caudate nucleus, and/or a tissue of the putamen. In some embodiments, the target tissue or tissues may comprise a muscle tissue. In certain embodiments, the target tissue or tissues may comprise a skeletal muscle tissue, a cardiac tissue, and/or a smooth muscle tissue. In some embodiments, the target tissue or tissues may comprise an endocrine tissue. In certain embodiments, the target tissue or tissues may comprise a pancreatic tissue and/or an adipose tissue. In some embodiments, the target tissue or tissues may comprise a tissue of the immune system. In certain embodiments, the target tissue or tissues may comprise a lymphoid and/or myeloid tissue. In particular embodiments, the target tissue or tissues may comprise a tissue of the bone marrow, a thymic tissue, a lymph node tissue, a spleen tissue, a tissue of the tonsil, and/or a tissue of the Peyer's patches.

### Target Sequences

In one aspect, a target gene according to the present disclosure may be the huntingtin gene. In some embodiments, the target gene may be HTT. In certain embodiments, the target gene may be mutant HTT. In certain embodiments, the target gene may be mutant HTT comprising expanded CAG repeats. In certain embodiments, the target gene may be mutant HTT comprising more than 35 CAG repeats in exon 1.

In some embodiments, the target gene may comprise extra CAG repeats added to the sequence of the gene located on chromosome 4, with gene location 4p16.3 at nucleotide positions 3074681 to 3243960 (according to Gene Assembly GRCh38.p13), which encodes 67 exons (NCBI, Gene ID: 3064). In some embodiments, the target gene may comprise extra CAG repeats added to the sequence of NCBI Reference Sequence: NC_000004.12.

### Target Sequences

In one aspect, any parts of a target gene according to the present disclosure may be targeted (i.e., may be a target sequence), and the target sequence may be any parts of the sequence of the coding (sense) strand or the non-coding (antisense) strand of the gene or its transcript (including pre- and post-splicing sequences), any parts of the sequence of the coding region or non-coding region of the gene or its transcripts, or any parts of the sequence of the polynucleotide regions regulating the expression of the gene (e.g., promoter region, enhancer region, transcription factor-binding site). In some embodiments, the target sequence may be any parts of the gene, wherein editing thereof results in reduced expression of the target gene or suppression or inhibition of the pathogenic function(s) of the target gene.

In some embodiments, the target sequence may be comprised in HTT, the gene located on chromosome 4, with gene location 4p16.3 at nucleotide positions 3074681 to 3243960 (according to Gene Assembly GRCh38.p13) (NCBI, Gene ID: 3064). In some embodiments, the target sequence may be comprised in the sequence of NCBI Reference Sequence: NC_000004.12.

In some embodiments, the target sequence may be comprised in exon 5 of HTT or in its proximity (e.g., within ˜100 nucleotides) within HTT. In certain embodiments, the target sequence may be comprised in the sequence corresponding to the nucleotide positions 3105357 to 3105436 of HTT or in its proximity (e.g., within ˜100 nucleotides) within HTT. In certain embodiments, the target sequence may be comprised in SEQ ID NO: 200, 201, or 202, or the reverse complement sequences thereof (SEQ ID NO: 205, 206, or 207, respectively). In particular embodiments, the target sequence may be a sequence that is reverse complementary to any of SEQ ID NOS: 4-6, 210-213, 220-223, and 230-233.

In some embodiments, the target sequence may be comprised in exon 8 of HTT or in its proximity (e.g., within ˜100 nucleotides) within HTT. In certain embodiments, the target sequence may be comprised in the sequence corresponding to the nucleotide positions 3116085 to 3116263 of HTT or in its proximity (e.g., within ˜100 nucleotides) within HTT. In certain embodiments, the target sequence may be comprised in SEQ ID NO: 300, 301, or 302, or the reverse complement sequences thereof (SEQ ID NO: 305, 306, or 307, respectively). In particular embodiments, the target sequence may be a sequence that is reverse complementary to any of SEQ ID NOS: 7-9, 310-313, 320-323, and 330-333.

In some embodiments, the target sequence may be comprised in exon 31 of HTT or in its proximity (e.g., within ˜100 nucleotides) within HTT. In certain embodiments, the target sequence may be comprised in the sequence corresponding to the nucleotide positions 3172908 to 3173131 of HTT or in its proximity (e.g., within ˜100 nucleotides) within HTT. In certain embodiments, the target sequence may be comprised in SEQ ID NO: 400, 401, or 402, or the reverse complement sequences thereof (SEQ ID NO: 405, 406, or 407, respectively). In particular embodiments, the target sequence may be a sequence that is reverse complementary to any of SEQ ID NOS: 1-3, 410-413, 420-423, and 430-433.

In some embodiments, the target sequence may be comprised in mouse huntingtin gene (Htt), the gene located on chromosome 5, with gene location 5 B2; 5 17.92 cM at nucleotide positions 34919084 to 35069878 (according to Gene Assembly GRCm39), which encodes 67 exons (NCBI, Gene ID: 15194). In some embodiments, the target sequence may be comprised in the sequence of NCBI Reference Sequence: NC_000071.7.

In some embodiments, the target sequence may be comprised in exon 31 of Htt or in its proximity (e.g., within ˜100 nucleotides) within Htt. In certain embodiments, the target sequence may be comprised in the sequence corresponding to the nucleotide positions 35004798 to 35005021 of Htt or in its proximity (e.g., within ˜100 nucleotides) within Htt. In certain embodiments, the target sequence may be comprised in SEQ ID NO: 500 or 501, or the reverse complement sequences thereof.

### Guide RNAs, Polynucleotide, and Vectors

In one aspect, the present disclosure provides gRNAs, which may be for a CRISPR-mediated gene editing reporter system, polynucleotides encoding such a gRNA, and vectors comprising such a polynucleotide.

### Targeting Sequence Lengths

While 20 nt is the most commonly used length for a targeting sequence of a gRNA in the field, truncated targeting sequences (e.g., 19, 18, or 17 nt in length) are also known to provide similar or equivalent, or in some cases better gene editing effects (Fu et al. Nat Biotechnol. 2014 March; 32(3):279-284.). Therefore, in some embodiments, a gRNA may have a crRNA or a crRNA sequence comprising a targeting sequence comprising at least 17 nucleotides. It was further shown that longer targeting sequences (e.g., targeting sequences up to 30 nt in length) may be also used because longer gRNAs may be cleaved before participating in gene editing activities, so that the targeting sequence may be e.g., 20 nt in length (Ran et al., Cell. 2013 Sep. 12;154(6):1380-9). Therefore, in some embodiments, the length of a targeting sequence may be 17, 18, 19, 20, 21, 22, 23, 24, 25, 26, 27, 28, 29, or 30 nt.

### Targeting Sequences

In one aspect, the targeting sequence may be reverse complementary to any of the target sequences described above.

In some embodiments, gRNAs for effecting CRISPR-mediated gene editing may be designed based on the sequence of the target gene and the PAM sequence recognized by the Cas nuclease to be used.

When Cas9 of Streptococcus pyogenes is used, a targeting sequence may be designed, for example, as the 20 (or alternatively about 17-24) nucleotides immediately upstream (the 5′-side) of any of the 5′-NGG-3′ (N may be any nucleic acid) PAM sites present in the target gene (the coding strand or non-coding strand).

When Cpf1 is used, a targeting sequence may be designed, for example, as the 20 (or alternatively about 17-24) nucleotides immediately downstream (the 3′-side) of any of the 5′-TTTN-3′ (N may be any nucleic acid) PAM sites present in the target gene (the coding strand or non-coding strand). A desired targeting sequence may be selected from all possible sequences, for example based on, the proximity to the desired editing position, the G-C content (e.g., for example in the range of about 40-80%), self-complementarity, the potential editing efficiency, and/or the potential off-target effects. Non-limiting tools for selecting a desired target-complementary sequence include CHOPCHOP (https://chopchop.cbu.uib.no/).

### Targeting Sequences for HTT Exon 5

In some embodiments, the targeting sequence may comprise or consist of (i) SEQ ID NO: 4, 210, 211, 212, or 213; (ii) a sequence comprising one or more mutations relative to SEQ ID NO: 4, 210, 211, 212, or 213, optionally at position(s) other than the 4th to the 7th nucleotide positions from the 3′-end of SEQ ID NO: 4, 210, 211, 212, or 213; or (iii) a sequence comprising at least 85, 90, 95, 96, 97, 98 or 99% sequence identity to SEQ ID NO: 4, 210, 211, 212, or 213.

In some embodiments, the targeting sequence may comprise or consist of (i) SEQ ID NO: 5, 220, 221, 222, or 223; (ii) a sequence comprising one or more mutations relative to SEQ ID NO: 5, 220, 221, 222, or 223, optionally at position(s) other than the 4th to the 7th nucleotide positions from the 3′-end of SEQ ID NO: 5, 220, 221, 222, or 223; or (iii) a sequence comprising at least 85, 90, 95, 96, 97, 98 or 99% sequence identity to SEQ ID NO: 5, 220, 221, 222, or 223.

In some embodiments, the targeting sequence may comprise or consist of (i) SEQ ID NO: 6, 230, 231, 232, or 233; (ii) a sequence comprising one or more mutations relative to SEQ ID NO: 6, 230, 231, 232, or 233, optionally at position(s) other than the 4th to the 7th nucleotide positions from the 3′-end of SEQ ID NO: 6, 230, 231, 232, or 233; or (iii) a sequence comprising at least 85, 90, 95, 96, 97, 98 or 99% sequence identity to SEQ ID NO: 6, 230, 231, 232, or 233.

### Targeting Sequences for HTT Exon 8

In some embodiments, the targeting sequence may comprise or consist of (i) SEQ ID NO: 7, 310, 311, 312, or 313; (ii) a sequence comprising one or more mutations relative to SEQ ID NO: 7, 310, 311, 312, or 313, optionally at position(s) other than the 4th to the 7th nucleotide positions from the 3′-end of SEQ ID NO: 7, 310, 311, 312, or 313; or (iii) a sequence comprising at least 85, 90, 95, 96, 97, 98 or 99% sequence identity to SEQ ID NO: 7, 310, 311, 312, or 313.

In some embodiments, the targeting sequence may comprise or consist of (i) SEQ ID NO: 8, 320, 321, 322, or 323; (ii) a sequence comprising one or more mutations relative to SEQ ID NO: 8, 320, 321, 322, or 323, optionally at position(s) other than the 4th to the 7th nucleotide positions from the 3′-end of SEQ ID NO: 8, 320, 321, 322, or 323; or (iii) a sequence comprising at least 85, 90, 95, 96, 97, 98 or 99% sequence identity to SEQ ID NO: 8, 320, 321, 322, or 323.

In some embodiments, the targeting sequence may comprise or consist of (i) SEQ ID NO: 9, 330, 331, 332, or 333; (ii) a sequence comprising one or more mutations relative to SEQ ID NO: 9, 330, 331, 332, or 333, optionally at position(s) other than the 4th to the 7th nucleotide positions from the 3′-end of SEQ ID NO: 9, 330, 331, 332, or 333; or (iii) a sequence comprising at least 85, 90, 95, 96, 97, 98 or 99% sequence identity to SEQ ID NO: 9, 330, 331, 332, or 333.

### Targeting Sequences for HTT Exon 31

In some embodiments, the targeting sequence may comprise or consist of (i) SEQ ID NO: 1, 410, 411, 412, or 413; (ii) a sequence comprising one or more mutations relative to SEQ ID NO: 1, 410, 411, 412, or 413, optionally at position(s) other than the 4th to the 7th nucleotide positions from the 3′-end of SEQ ID NO: 1, 410, 411, 412, or 413; or (iii) a sequence comprising at least 85, 90, 95, 96, 97, 98 or 99% sequence identity to SEQ ID NO: 1, 410, 411, 412, or 413.

In some embodiments, the targeting sequence may comprise or consist of (i) SEQ ID NO: 2, 420, 421, 422, or 423; (ii) a sequence comprising one or more mutations relative to SEQ ID NO: 2, 420, 421, 422, or 423, optionally at position(s) other than the 4th to the 7th nucleotide positions from the 3′-end of SEQ ID NO: 2, 420, 421, 422, or 423; or (iii) a sequence comprising at least 85, 90, 95, 96, 97, 98 or 99% sequence identity to SEQ ID NO: 2, 420, 421, 422, or 423.

In some embodiments, the targeting sequence may comprise or consist of (i) SEQ ID NO: 3, 430, 431, 432, or 433; (ii) a sequence comprising one or more mutations relative to SEQ ID NO: 3, 430, 431, 432, or 433, optionally at position(s) other than the 4th to the 7th nucleotide positions from the 3′-end of SEQ ID NO: 3, 430, 431, 432, or 433; or (iii) a sequence comprising at least 85, 90, 95, 96, 97, 98 or 99% sequence identity to SEQ ID NO: 3, 430, 431, 432, or 433.

In alternative embodiments, the targeting sequence may comprise or consist of (i) SEQ ID NO: 440, 441, 442, or 443; (ii) a sequence comprising one or more mutations relative to SEQ ID NO: 440, 441, 442, or 443, optionally at position(s) other than the 4th to the 7th nucleotide positions from the 3′-end of SEQ ID NO: 440, 441, 442, or 443; or (iii) a sequence comprising at least 85, 90, 95, 96, 97, 98 or 99% sequence identity to SEQ ID NO: 440, 441, 442, or 443.

In further alternative embodiments, the targeting sequence may comprise or consist of (i) SEQ ID NO: 450, 451, 452, or 453; (ii) a sequence comprising one or more mutations relative to SEQ ID NO: 450, 451, 452, or 453, optionally at position(s) other than the 4th to the 7th nucleotide positions from the 3′-end of SEQ ID NO: 450, 451, 452, or 453; or (iii) a sequence comprising at least 85, 90, 95, 96, 97, 98 or 99% sequence identity to SEQ ID NO: 450, 451, 452, or 453.

sRNA Structure/Type

In some embodiments, a gRNA according to the present disclosure may be a sgRNA which is a single RNA molecule comprising a crRNA sequence comprising a targeting sequence and a rRNA backbone sequence. In certain embodiments, the crRNA sequence and the tracrRNA sequence may be linked via a linker optionally comprising SEQ ID NO: 109. In certain embodiments, the targeting sequence may be followed by, optionally immediately followed by, a sgRNA backbone sequence of any of SEQ ID NOS: 111-114. In certain embodiments, the sgRNA backbone sequence may be followed by one or more uracils, e.g., about 4-10 uracils.

hi some embodiments, a gRNA according to the present disclosure may be a dgRNA which is a complex formed by (i) a crRNA molecule comprising a targeting sequence and (ii) a tracrRNA molecule. In certain embodiments, the targeting sequence may be followed by, optionally immediately followed by, a sgRNA backbone sequence, which may be SEQ ID NOS: 115; and the tracrRNA may comprise SEQ ID NO: 116. In certain embodiments, the targeting sequence may be followed by, optionally immediately followed by, a sgRNA backbone sequence, which may be SEQ ID NOS: 117; and the tracrRNA may comprise SEQ ID NO: 118.

gRNA Modifications

In some embodiments, a gRNA according to the present disclosure may comprise a modification. In some embodiments, the modification may be selected from the group consisting of: 2′-O-C1-4alkyl such as 2′-O-methyl (2′-OMe), 2′-deoxy (2′-H), 2′-O—C1-3alkyl-O-C1-3alkyl such as 2′-methoxyethyl (2′-MOE), 2′-fluoro (2′-F), 2′-amino (2′-NH2), 2′-arabinosyl (2′-arabino) nucleotide, 2′F-arabinosyl (2-F-arabino) nucleotide, 2′-locked nucleic acid (LNA) nucleotide, 2′-unlocked nucleic acid (ULNA) nucleotide a sugar in 1 form (1-sugar), and 4′-thioribosyl nucleotide. In some embodiments, the modification is an internucleotide linkage modification selected from the group consisting of: phosphorothioate, phosphonocarboxylate, thiophosphonocarboxylate, alkylphosphonate, and phosphorodithioate. In some embodiments, the modification is selected from the group consisting of: 2-thiouracil (2-thioU), 2-thiocytosine (2-thioC), 4-thiouracil (4-thioU), 6-thioguanine (6-thioG), 2-aminoadenine (2-aminoA), 2-aminopurine, pseudouracil, hypoxanthine, 7-deazaguanine, 7-deaza-8-azaguanine. 7-deazaadenine, 7-deaza-8-azaadenine, 5-methylcytosine (5-methylC), 5-methyluracil (5-methylU), 5-hydroxymethylcytosine, 5-hydroxymethyluracil, 5,6-dehydrouracil, 5-propynylcytosine, 5-propynyluracil, 5-ethnylcytosine, 5-ethynyluracil, 5-allyluracil (5-allylU), 5-allylcytosine (5-allylC), 5-aminoallyluracil (5-aminoallylU), 5-aminoallyl-cytosine (5-aminoallylC), an abasic nucleotide, Z base, P base, Unstructured Nucleic Acid (UNA), isoguanine (isoG), isocytosine (isoC), and 5-methyl-2-pyrimidine.

In particular embodiments, the gRNA may comprise: (i) 2-O-methylation further optionally at first three and last three bases, and/or (ii) one or more 3′ phosphorothioate bonds, further optionally between first three and last two bases.

### Polynucleotides and Vectors

Polynucleotides encoding a gRNA according to the present disclosure are also provided. Such a polynucleotide may encode one or more gRNAs. When a gRNA is a dgRNA (i.e., comprising a crRNA and a tracrRNA), a crRNA and a tracrRNA may be encoded by separate polynucleotides, i.e., two polynucleotides (one encoding a crRNA and one encoding a tracrRNA) may together encode one gRNA.

Vectors comprising such a polynucleotide encoding such a gRNA according to the present disclosure are further provided. Such a vector may comprise one or more polynucleotides each encoding one or more gRNAs. When a gRNA is a dgRNA, a crRNA and a tracrRNA may be encoded in a single vector (one vector comprising both a polynucleotide encoding a crRNA and a polynucleotide encoding a tracrRNA) or separate vectors (one comprising a polynucleotide encoding a crRNA and one comprising a polynucleotide encoding a tracrRNA).

### RNPs

In one aspect, the present disclosure provides RNPs (single RNP or a mixture of different RNPs) comprising one or more gRNAs and a Cas endonuclease. Such an RNP may be used as part of a CRISPR-mediated gene editing reporter system.

gRNA Combinations

In some embodiments, the one or more gRNAs comprised in one or more RNPs according to the present disclosure may be any one or more of the gRNAs described above.

In certain embodiments, the one or more gRNA comprised in one or more RNPs may target HTT exon 5. In certain embodiments, the one or more gRNA comprised in one or more RNPs may comprise three or more gRNAs specific for HTT exon 5. In certain embodiments, the one or more gRNA comprised in one or more RNPs may comprise the targeting sequence(s) of: (a) SEQ ID NO: 4, 210, 211, 212, or 213; (b) SEQ ID NO: 5, 220, 221, 222, or 223; and/or (c) SEQ ID NO: 6, 230, 231, 232, or 233. In particular embodiments, the one or more gRNA comprised in one or more RNPs may comprise three gRNAs: (a) one comprising the targeting sequence of SEQ ID NO: 210; (b) one comprising the targeting sequence of SEQ ID NO: 220; and (c) one comprising the targeting sequence of SEQ ID NO: 230. In particular embodiments, a RNP may comprise a gRNA comprising the targeting sequence of SEQ ID NO: 210. In particular embodiments, a RNP may comprise a gRNA comprising the targeting sequence of SEQ ID NO: 220. In particular embodiments, a RNP may comprise a gRNA comprising the targeting sequence of SEQ ID NO: 230.

In certain embodiments, the one or more gRNA comprised in one or more RNPs may target HTT exon 8. In certain embodiments, the one or more gRNA comprised in a one or more RNPs may comprise three or more gRNAs specific for HTT exon 8. In certain embodiments, the one or more gRNA comprised in one or more RNPs may comprise the targeting sequence(s) of: (a) SEQ ID NO: 7, 310, 311, 312, or 313; (b) SEQ ID NO: 8, 320, 321, 322, or 323; and/or (c) SEQ ID NO: 9, 330, 331, 332, or 333. In particular embodiments, the one or more gRNA comprised in one or more RNPs may comprise three gRNAs: (a) one comprising the targeting sequence of SEQ ID NO: 310; (b) one comprising the targeting sequence of SEQ ID NO: 320; and (c) one comprising the targeting sequence of SEQ ID NO: 330. In particular embodiments, a RNP may comprise a gRNA comprising the targeting sequence of SEQ ID NO: 310. In particular embodiments, a RNP may comprise a gRNA comprising the targeting sequence of SEQ ID NO: 320. In particular embodiments, a RNP may comprise a gRNA comprising the targeting sequence of SEQ ID NO: 330.

In certain embodiments, the one or more gRNA comprised in one or more RNPs may target HTT exon 31. In certain embodiments, the one or more gRNA comprised in a one or more RNPs may comprise three or more gRNAs specific for HTT exon 31. In certain embodiments, the one or more gRNA comprised in one or more RNPs may comprise the targeting sequence(s) of: (a) SEQ ID NO: 1, 410, 411, 412, or 413; (b) SEQ ID NO: 2, 420, 421, 422, or 423; and/or (c) SEQ ID NO: 3, 430, 431, 432, or 433. In certain embodiments, in (b), SEQ ID NO: 440, 441, 442, or 443 or alternatively SEQ ID NO: 450, 451, 452, or 453 may be used instead. In particular embodiments, the one or more gRNA comprised in one or more RNPs may comprise three gRNAs: (a) one comprising the targeting sequence of SEQ ID NO: 410; (b) one comprising the targeting sequence of SEQ ID NO: 420; and (c) one comprising the targeting sequence of SEQ ID NO: 430. In particular embodiments, a RNP may comprise a gRNA comprising the targeting sequence of SEQ ID NO: 410. In particular embodiments, a RNP may comprise a gRNA comprising the targeting sequence of SEQ ID NO: 420. In particular embodiments, a RNP may comprise a gRNA comprising the targeting sequence of SEQ ID NO: 430.

### Cas Endonuclease

In some embodiments, a Cas endonuclease comprised in a RNP may be any of the Cas endonucleases described herein. In certain embodiments, a Cas endonuclease may be Cas9. In particular embodiments, a Cas endonuclease may be SpCas9, optionally having SEQ ID NO: 600, or a variant SpCas9, optionally comprising any of SEQ ID NOS: 601-611.

### RNP Formation

In some embodiments, a RNP may be formed by mixing a solution comprising a gRNA (one gRNA or a mixture of different gRNAs, e.g., three gRNAs) and a solution comprising a Cas endonuclease at an approximately equimolar ratio. In certain embodiments, the mixing may be for about 5 minutes. In certain embodiments, the solution comprising a gRNA may have a pH of about 6 to 8, about 6.5 to 7.5, or optionally about 7. In certain embodiments, the solution comprising a Cas endonuclease may have a pH of about 6 to 8, about 6.5 to 7.5, or optionally about 7 In certain embodiments, the resulting solution comprising a RNP may comprise a pH of about 6 to 8, about 6.5 to 7.5, or optionally about 7.

### Compositions

In one aspect, the present disclosure provides compositions comprising one or more RNPs and a pharmaceutically acceptable carrier, and optionally, one or more template DNAs. The one or more RNPs may comprise or consist of any of the RNP or RNPs described herein.

### RNP Combinations

In some embodiments, the RNPs may comprise a set of three RNPs targeting HTT exon 5, according to any of the exon 5 targeting RNPs described herein. In particular embodiments, the gRNAs comprised in the three RNP may respectively comprise the targeting sequences of: (a) SEQ ID NO: 210, 211, 212, or 213; (b) SEQ ID NO: 220, 221, 222, or 223; and (c) SEQ ID NO: 230, 231, 232, or 233.

In some embodiments, the RNPs may comprise a set of three RNPs targeting HTT exon 8, according to any of the exon 8 targeting RNPs described herein. In particular embodiments, the gRNAs comprised in the three RNP may respectively comprise the targeting sequences of: (a) SEQ ID NO: 310, 311, 312, or 313; (b) SEQ ID NO: 320, 321, 322, or 323; and (c) SEQ ID NO: 330, 331, 332, or 333.

In some embodiments, the RNPs may comprise a set of three RNPs targeting HTT exon 31, according to any of the exon 31 targeting RNPs described herein. In particular embodiments, the gRNAs comprised in the three RNP may respectively comprise the targeting sequences of: (a) SEQ ID NO: 410, 411, 412, or 413; (b) SEQ ID NO: 420, 421, 422, or 423; and (c) SEQ ID NO: 430, 431, 432, or 433. In alternative embodiments, in (b), SEQ ID NO: 440, 441, 442, or 443 or SEQ ID NO: 450, 451, 452, or 453 may be used instead.

In another aspect, the present disclosure provides compositions comprising (A) a pharmaceutically acceptable carrier; and (B) (a) one or more isolated gRNAs according to any of the gRNAs described herein or one or more polynucleotides encoding the one or more isolated gRNAs, and (b) a Cas endonuclease or a polynucleotide encoding a Cas endonuclease; and optionally (C) one or more template DNAs or one or more polynucleotides encoding one or more template DNAs.

### RRNA Combinations

In some embodiments, the one or more isolated gRNAs may target HTT exon 5. In certain embodiments, the one or more isolated gRNAs may be any of the gRNA combinations for targeting HTT exon 5 as described herein.

In some embodiments, the one or more isolated gRNAs may target HTT exon 8. In certain embodiments, the one or more isolated gRNAs may be any of the gRNA combinations for targeting HTT exon 8 as described herein.

In some embodiments, the one or more isolated gRNAs may target HTT exon 31. In certain embodiments, the one or more isolated gRNAs may be any of the gRNA combinations for targeting HTT exon 31 as described herein.

In some embodiments, the one or more isolated gRNAs may target more than one of exons 5, 8, and 31 of HTT. In certain embodiments, the one or more isolated gRNAs may be a combination of gRNAs for targeting HTT exon 5, 8, and 31 as described herein.

### Cas Endonuclease

In some embodiments, a Cas endonuclease comprised in a RNP may be any of the Cas endonucleases described herein. In certain embodiments, a Cas endonuclease may be Cas9. In particular embodiments, a Cas endonuclease may be SpCas9, optionally having SEQ ID NO: 600, or a variant SpCas9, optionally comprising any of SEQ ID NOS: 601-611.

### Vectors

The vectors that may encode one or more gRNAs and/or a Cas endonuclease in some embodiments of a composition may be any appropriate vectors. In certain embodiments, the vectors may be individually selected from plasmids, RNA replicons, virus-like particles (VLPs), and viral vectors, optionally retroviral, lentiviral, or adenoviral vectors.

### Template DNAs

Any of the compositions according to the present disclosure may comprise one or more template DNA, which may facilitate rejoining of gene segments cleaved by CRISPR-mediated gene editing.

In some embodiments, the template DNA may be a ssODN or a double strand DNA. In some embodiments, the ssODN may comprise a 5′ homology arm and a 3′ homology arm and, optionally, also a central region (e.g., may vary in length, such as 1-100 nt) between the homology arms.

In some embodiments, when a given gene segment is cleaved at two sites, the ssODN may be designed to join the two cleavage sites, thereby removing the intervening sequence between the two cleavage sites.

In some embodiments, a ssODN may be designed to join two cleavage sites within HTT exon 5. In certain embodiments, the ssODN may be designed to join two cleavage sites: of (i) between the 9th and 10^{th} nucleotides from the 5′ end of SEQ ID NO: 200 and (ii) between the 72th and 73th nucleotides from the 5′ end of SEQ ID NO: 200; and/or of (iii) between the 9th and 10^{th} nucleotides from the 3′ end of SEQ ID NO: 205 and (iv) between the 72th and 73th nucleotides from the 3′ end of SEQ ID NO: 200.

In certain embodiments, the 5′ homology arm may comprise the sequence corresponding to the first nucleotide to at least the 10th (up to 100th) nucleotide counting from the 3′-end of SEQ ID NO: 268. In certain embodiments, the 3′ homology arm may comprise the sequence corresponding to the first nucleotide to at least the 10th (up to 100th) nucleotide counting from the 5′-end of SEQ ID NO: 278.

In particular embodiments, the 5′-homology arm may comprise any of SEQ ID NOS: 261-268 and the 3′-homology arm may comprise any of SEQ ID NOS: 271-278. In particular embodiments, the ssODN may comprise any of SEQ ID NOS: 281-288.

In some embodiments, a ssODN may be designed to join two cleavage sites within HTT exon 8. In certain embodiments, the ssODN may be designed to join two cleavage sites: of (i) between the 15th and 16th nucleotides from the 5′ end of SEQ ID NO: 300 and (ii) between the 112th and 113th nucleotides from the 5′ end of SEQ ID NO: 300; and/or of (iii) between the 15th and 16th nucleotides from the 3′ end of SEQ ID NO: 305 and (iv) between the 112th and 113th nucleotides from the 3′ end of SEQ ID NO: 305.

In certain embodiments, the 5′ homology arm may comprise the sequence corresponding to the first nucleotide to at least the 10th (up to 100th) nucleotide counting from the 3′-end of SEQ ID NO: 368. In certain embodiments, the 3′ homology arm may comprise the sequence corresponding to the first nucleotide to at least the 10th (up to 100th) nucleotide counting from the 5′-end of SEQ ID NO: 378.

In particular embodiments, the 5′-homology arm may comprise any of SEQ ID NOS: 361-368 and the 3′-homology arm may comprise any of SEQ ID NOS: 371-378. In particular embodiments, the ssODN may comprise any of SEQ ID NOS: 381-388.

In some embodiments, a ssODN may be designed to join two cleavage sites within HTT exon 31. In certain embodiments, the ssODN may be designed to join two cleavage sites: of (i) between the 36th and 37th nucleotides from the 5′ end of SEQ ID NO: 400 and (ii) between the 185th and 186th nucleotides from the 5′ end of SEQ ID NO: 400; and/or of (iii) between the 36th and 37th nucleotides from the 3′ end of SEQ ID NO: 405 and (iv) between the 185th and 186th nucleotides from the 3′ end of SEQ ID NO: 405.

In certain embodiments, the 5′ homology arm may comprise the sequence corresponding to the first nucleotide to at least the 10th (up to 100th) nucleotide counting from the 3′-end of SEQ ID NO: 468. In certain embodiments, the 3′ homology arm may comprise the sequence corresponding to the first nucleotide to at least the 10th (up to 100th) nucleotide counting from the 5′-end of SEQ ID NO: 478.

In particular embodiments, the 5′-homology arm may comprise any of SEQ ID NOS: 461-468 and the 3′-homology arm may comprise any of SEQ ID NOS: 471-478. In particular embodiments, the ssODN may comprise any of SEQ ID NOS: 481-488.

In further embodiments, any ssODN comprising the reverse complementary sequence of any of the above-described sequences may be used. In yet further embodiments, any double stranded DNA comprising a first strand having any of the above-described sequences and a second strand which is complementary to the first strand may be used. In additional embodiments, one or more mutations may be introduced to any of the ssODNs or double strand DNAs.

### Pharmaceutically Acceptable Carrier

The pharmaceutically acceptable carrier comprised in any of the compositions according to the present invention may be any carrier that allows for and/or compatible with administration of the CRISPR-mediated gene editing agent(s) (e.g., any of the gRNA(s), Cas, and/or one or more polynucleotides or vectors encoding the same as described herein) in a subject.

In some embodiments, the carrier may be a lipid-based TCV. In certain embodiments, the TCV may comprise an ionizable cationic lipid (e.g., DODMA), a phospholipid and/or a helper lipid (e.g., DOPE, DSPC), and cholesterol. In particular embodiments, the TCV may comprise DODMA, DOPE, DSPC, and cholesterol at the 20:30:10:40 mol % ratio. In particular embodiments, the TCV may be a TCV produced by mixing a solution of DODMA:DOPE:DSPC:Cholesterol=20:30:10:40 mol % in ethanol with an aqueous solution of about pH4, such as 25 mM acetate buffer. The solution of TCVs formed may be subject to an ethanol removal step, e.g., dialysis against a buffer not containing ethanol.

When the carrier is a lipid-based TCV, one or more of the components of the composition may be encapsulated in the TCV. In some embodiments, one or more RNPs may be encapsulated in a TCV. In certain embodiments, the encapsulation may be performed by mixing a solution of TCV and a solution of RNP. In particular embodiments, a RNP solution (which may be at about pH7) may be mixed into a solution of TCV at pH 4 (e.g., TCVs contained in an about 25 mM acetate buffer). In certain embodiments, the mixing may be performed in in the absence of ethanol, methanol, isopropanol, THF, DMSO, DMF, and ACN. In certain embodiments, the mixing may be performed in the absence of organic solvents. In certain embodiments, the mixing may be performed in the absence of SDS. In certain embodiments, the mixing may be performed in the absence of detergents. In certain embodiments, the mixing may be performed in the absence of destabilizing agents.

### Other Components of Compositions

In some embodiments, the composition according to the present disclosure may contain a very low level of, substantially free of, essentially free of, or entirely free ethanol. In certain embodiments, the ethanol concentration may be 5% (v/v) or below. In particular embodiments, the ethanol concentration may be 0.5% (v/v). In some embodiments, the composition according to the present disclosure may be substantially, essentially, or entirely free of ethanol, methanol, isopropanol, THF, DMSO, DMF, and ACN. In certain embodiments, the composition may be substantially, essentially, or entirely free of organic solvents. In some embodiments, the composition according to the present disclosure may be substantially, essentially, or entirely free of SDS. In certain embodiments, the composition may be substantially, essentially, or entirely free of detergents. In certain embodiments, the composition may be substantially, essentially, or entirely free of organic solvents and detergents. In certain embodiments, the composition may be substantially, essentially, or entirely free of destabilizing agents.

In some embodiments, the composition according to the present disclosure may further comprise one or more agents for preventing, ameliorating, slowing the progression of, and/or treating HD. Agents that may be comprised may include but not be limited to an agent for controlling HD symptoms such as those described herein, including motor symptoms, psychiatric symptoms, and cognitive symptoms.

### Methods

In one aspect, the present disclosure provides method of effecting CRISPR-mediated gene editing of the huntingtin gene (e.g., HTT) in one or more target cells and/or one or more target tissues. The method may be performed, for example, in vitro or ex vivo. Such a method may comprise applying any of the compositions described herein to the one or more cells or one or more tissues. In some embodiments, the method may further comprise analyzing the level of CRISPR-mediated gene editing events in said one or more target cells or said one or more target tissues. In certain embodiments, the method may be for assessing the effect of huntingtin gene editing on a given target cell and/or target tissue.

### Target Cells

The target cell(s) may comprise any of the target cells described above and herein. In some embodiments, the target cell(s) may comprise a cell(s) of the brain, for example, a neural cell, a cortical neuron, a cell of the basal ganglia, a cell of the striatum, a cell of the caudate nucleus, and/or a cell of the putamen. In some embodiments, the target cell(s) may comprise a cell(s) of the muscle, a myocyte, for example, a skeletal myocyte, a cardiomyocyte, and/or a smooth myocyte. In some embodiments, the target cell(s) may comprise a cell(s) of an endocrinal system, for example, a pancreatic cell and/or an adipocyte. In some embodiments, the target cells may comprise a cell(s) of the immune system or a blood cell(s), for example, a lymphocyte, a macrophage, and/or a monocyte. In some embodiments, the target cell or target cells may comprise a fibroblast(s) and/or a lymphoblast(s). In particular embodiments, the target cell or target cells may comprise a cell(s) of the striatum.

In some embodiments, the target cell(s) may comprise or consist of a mammalian cell(s), a human cell(s), a cell line, a stem cell-derived cell(s), an induced pluripotent stem cell (iPSC)-derived cell(s), a primary cell(s), and/or a cell(s) derived from a subject who has or has a risk of developing HD;

### Target Tissues

The target tissue(s) may comprise any of the target tissues described above and herein. In some embodiments, the target tissue(s) may comprise a CNS tissue. In certain embodiments, the target tissue(s) may comprise a brain tissue and/or a nervous tissue, for example, a tissue of the basal ganglia, the striatum, the caudate nucleus, and/or the putamen. In some embodiments, the target tissue(s) may comprise a muscle tissue, for example, a skeletal muscle tissue, a cardiac tissue, and/or a smooth muscle tissue. In some embodiments, the target tissue(s) may comprise an endocrine tissue, for example, a pancreatic tissue and/or an adipose tissue. In some embodiments, the target tissue(s) may comprise a tissue of the immune system, for example, a lymphoid and/or myeloid tissue. In particular embodiments, the target tissue(s) may comprise a tissue of the bone marrow, a thymic tissue, a lymph node tissue, a spleen tissue, a tissue of the tonsil, and/or a tissue of the Peyer's patches.

In one aspect, the present disclosure provides method of effecting CRISPR-mediated gene editing of the huntingtin gene (e.g., HTT) in one or more target cells and/or one or more target tissues in a subject. The method may be performed, for example, in vivo. Such a method may comprise administering any of the compositions described herein to the subject. The target cell(s) and/or target tissue(s) in the method may be according to any of the target cell(s) and/or target tissue(s) described herein.

In some embodiments, the administrating is effected so that a minimum of about 10%, about 15%, about 20%, about 30%, or an about final 15-30% or about final 20-40% target cells and/or tissue(s) with successful gene editing is achieved among the total target cells and/or tissue(s). In some embodiments, the method may be for preventing, ameliorating, slowing the progression of, and/or treating HD and/or one or more symptoms of HD in the subject. In some embodiments, the method may be for assessing the effect of the composition on the target cell(s) and/or target tissue(s). In some embodiments, the method may be for assessing the effect of the composition on the disease severity and/or progression of HD. In some embodiments, the method may be for assessing the effect of the composition on one or more HD symptoms, which may be any of the symptoms of HD, such as those specifically described herein. In certain embodiments, the method may be for comparing the effects of targeting different exons of the huntingtin gene, for example any of exons 5, 8, and 31, or comparing to the effects of targeting exon 1.

In some embodiments, the effect of the method may be evaluated based on various parameters. In certain embodiments, the evaluation may be based on any one or more of the following: the number of the target cells with successful gene editing; the % of the target cells with successful gene editing among total target cells; the % of the target tissue(s) with successful gene editing out of total target tissue(s); the expression level of the HTT gene in the one or more target cells; the expression level of the HTT gene in the one or more target tissues. In certain embodiments, the evaluation may be based on changes in the level of huntingtin protein aggregation, cell death, and/or inflammation in one or more of target cells and/or tissues. In certain embodiments, the evaluation may be based on changes in the mitochondrial function and/or caspase activity in one or more of target cells and/or tissues. In certain embodiments, the evaluation may be based on changes in the level of insulin in pancreatic cells and/or leptin release by adipose tissue.

In another aspect, the present disclosure provides methods of preventing, ameliorating, slowing the progression of, and/or treating HD and/or one or more symptoms of HD in a subject in need thereof. The method may comprise administering any of the compositions described herein to the subject. In certain embodiments, the method may be according to any of the in vivo methods described herein.

In some embodiments of the method, one or more agents for preventing, ameliorating, slowing the progression of, or treating HD may be further administered to the subject. Such agents may include but are not limited to drugs for controlling motor, cognitive, and/or psychiatric symptoms including the drugs specifically described herein.

In some embodiments, the HD symptom may be any one or more of the (a) cognitive deficits, such as (i) cognitive slowing, (ii) decreases in attention, and/or (iii) decreases in mental flexibility; (b) psychiatric disruption and/or emotional deficits, such as (i) depression, (ii) apathy, (iii) irritability, (iv) impulsivity, and/or (v) social disinhibition; (c) loss of motor coordination, such as (i) choreiform movements, (ii) gait disturbances, and/or (ii) bradykinesia, and/or (iv) rigidity; (d) weight loss; and/or (e) inflammation, optionally neural inflammation or inflammation in the brain.

In some embodiments, when one or more other agents in addition to a composition according to the present description is administered, the composition and the one or more other agents may be administered simultaneously or separately, using the same or different administration routes. When the composition and the one or more other agents are administered simultaneously, the composition and the one or more other agents may be contained in the same formulation or in separate formulations.

In certain embodiments, the effect of the method is evaluated based on changes in one or more symptoms of HD, which may be any HD symptoms, such as those described herein.

In any of the embodiments of any of the methods involving treatment of a subject described herein, the subject is a mammal. In certain embodiments, the subject may be a human subject, optionally a human subject who has or has a risk of developing HD. Alternatively, the subject may be a non-human subject, optionally a non-human primate or selected from a rodent (mouse, rat, guinea pig, hamster), rabbit, cat, dog, pig, goat, sheep, horse, or monkey, further optionally a mouse or a rat, in which case the method may be, for example, for assessing the effect of the composition.

In any of the embodiments of any of the methods involving treatment of a subject, the composition may be administered, for example, parenterally, locally, or enterally. In particular embodiments, the composition may be administered via direct injection into the CNS; intracranial injection; intraspinal injection, intrathecal injection, direct injection into the brain; direct injection into the basal ganglia; direct injection into the striatum; direct injection into the caudate nucleus and/or putamen. When another agent is separately administered from the compound, any appropriate administration route may be used for the other agent.

In any of the embodiments of any of the methods involving treatment of a subject, any appropriate amount and/or concentration of the gRNA(s) or the gRNA-encoding polynucleotide(s) may be contained in the composition. For example, a composition may comprise the gRNA(s) or the gRNA-encoding polynucleotide(s) at about 300 to 30000 nmol, optionally about 500 to 10000 nmol, about 1000 to 5000 nmol, about 2000 to 4000 nmol, about 2500 to 3000 nmol, or about 2700 nmol per mL.

In any of the embodiments of any of the methods involving treatment of a subject, any appropriate administration volume may be used. The appropriate volume may be determined based on the administration route. For example, the total volume comprising the composition for administration may be about 0.1-10000 μL, about 1-5000 μL, about 2-2000 μL, about 4-1000 μL, about 10-500 μL, about 20-200 μL.

In any of the embodiments of any of the methods involving treatment of a subject, the administration of the composition may occur only once or more. In some embodiments, the administration may occur twice or more, such as 3-5 times. In certain embodiments, the multiple administrations may occur with a certain interval, perhaps to allow for recovery of the subject from the procedures. Exemplary intervals may include about 1, 2, or 3 weeks, about 1, 2, 3, or 6 months, and/or about 1, 2, 3, or 6 years. In certain embodiments, the interval may be determined based on the effect of the method as evaluated as described herein, such as any of the following: changes in one or more of HD symptoms including but not limited to those described herein; changes in the huntingtin protein accumulation/aggregation; and/or changes in the number or % or target cells and/or tissues successfully undergone the intended gene editing.

In any of the embodiments of any of the methods involving treatment of a subject, the administration of the composition may occur at a single site or multiple sites. For example, when the composition is directly injected into the brain (e.g., striatum), the administrations may occur in each side of the brain.

In any of the embodiments of any of the methods involving treatment of a subject, the subject may be in any age groups and may or may not have developed HD yet. In some embodiments, the subject may be about 20 years old or older, about 25 years old or older, about 30 years old or older, about 35 years old or older, about 40 years old or older or juvenile. In some embodiments, the subject may have fully developed HD. In some embodiments, the subject may not have shown any HD symptoms. In some embodiments, the subject may be at a risk of developing HD. In some embodiments, the subject may be at an onset of HD.

Any of the compositions described herein may be used for performing any of the methods described herein. Furthermore, any of the gRNAs and any of the compositions described herein may be used for manufacturing a medicament, for example a medicament to be used for any of the methods described herein.

In some embodiments, a composition according to the present disclosure may be comprised in a kit. In certain embodiment, the kit may further comprise an instruction and/or a label, which optionally may be for used of the composition, optionally according to any of the methods described herein.

### Delivery Vehicles/Carriers

This section further describes variations related to delivery vehicles, particularly TCVs, applicable to any of the aspects and embodiments described herein.

### Vehicle Type

Any components that may be used for effecting gene editing as described herein may be carried into as a cargo (or cargoes) into a cell by a delivery vehicle. In some embodiments, such components for effecting gene editing may be comprised in a composition which comprises a pharmaceutically acceptable carrier. Such a carrier may be a delivery vehicle. Such a delivery vehicle may be a TCV.

### Lipid-Based TCVs

TCVs particularly used in the present disclosure include lipid-based TCVs. Compared to non-lipid-based TCVs such as viral vectors, lipid-based TCVs may have several advantages, e.g., less immunogenicity if needed, and no random integration into the target cell genome. Compositions and methods comprising or using any TCVs comprising or consisting of any appropriate lipids including those described herein at any appropriate ratio including ratios described herein are encompassed by the present invention. Compositions and methods comprising or using any TCVs produced according to any methods described herein are encompassed by the present invention.

### Cationic Lipid

In some embodiments, a lipid-based TCV may comprise at least one cationic lipid. In some embodiments, the at least one cationic lipid may comprise DODMA, DODAP, DLinDAP, KC2, MC3, DODAC, DDAB, DOTAP, DOTMA, DLinDMA, DLenDMA, DLin-C-DAP, DLin-DAC, DLin-MA, DLin-S-DMA, DLin-2-DMAP, DLin-TMA.Cl, DLin-TAR.Cl, DLin-MPZ, DLinAP, DOAP, DLin-EG-DMA, DLin-K-DMA, DLin-K-DMA or analogs thereof, ALNY-100, DOTMA, DOTAP.Cl, DC-Chol, DOSPA, DOGS″, DMRIE, or any combination thereof. In particular embodiments, the at least one ionizable cationic lipid may comprise or consist of DODMA. In some embodiments, the at least one cationic lipid may be an ionizable cationic lipid.

In some embodiments, a lipid-based TCV may comprise at least one ionizable cationic lipid. Examples of ionizable cationic lipids include but are not limited to: N-dimethyl-2,3-dioleyloxy)propylamine (DODMA), 1,2-dioleoyl-3-dimethylammonium propane (“DODAP”), 1,2-Dilinoleoyl-3-dimethylaminopropane (DLinDAP), 1,2-DiLinoleyloxy-N,N-dimethylaminopropane (DLinDMA), N,N-dimethyl-2,2-di-(9Z,12Z)-9,12-octadecadien-1-yl-1,3-dioxolane-4-ethanamine (KC2), and (6Z,9Z,28Z,31Z)-heptatriaconta-6,9,28,31-tetraen-19-yl 4-(dimethylamino)butanoate (MC3).

In some embodiments, a lipid-based TCV may be free of permanently cationic lipid. For example, N-(1-(2,3-dioleyloxyl)propyl)-N,N,N-trimethylammonium chloride (DOTMA), N-(1-(2,3-dioleyloxyl)propyl)-N-2-(sperminecarboxamido)ethyl)-N,N-dimethyl-ammonium trifluoroacetate (DOSPA) (which is a lipid component of Lipofectamine®), and N-(1-(2,3-dioleoyloxy)propyl)-N,N,N-trimethylammonium chloride (DOTAP) are permanently cationic lipids.

The amount of the at least one ionizable cationic lipid may be determined as appropriate. In some cases, the amount of the at least one ionizable cationic lipid to be used may be determined based on the type of cargo.

In some embodiments, the amount of ionizable cationic lipid(s) relative to the total amount of TCV components may be about 10 mol % to about 70 mol %. In some embodiments, the total amount of TCV components may be about 10 mol % to about 60 mol %, about 10 mol % to about 50 mol %, about 10 mol % to about 40 mol %, about 10 mol % to about 30 mol %, about 15 mol % to about 25 mol %, about 18 mol % to about 22 mol %, about 19 mol % to about 21 mol %, about 19.5 mol % to about 20.5 mol %, about 19.8 mol % to about 20.2 mol %, or about 20 mol %. In particular embodiments, for example when the cargo comprises a nucleic acid and a protein (or a RNP), the total amount of ionizable cationic lipid(s) relative to the total amount of TCV components may be about 20 mol %.

In a preferred embodiment, a lipid-based TCV according to the present disclosure may comprise DODMA at 20 mol % relative to the total amount of TCV components.

In some embodiments, the amount of ionizable cationic lipid(s) relative to the total amount of TCV components may be about 10 mol % to about 70 mol %, about 20 mol % to about 70 mol %, about 30 mol % to about 70 mol %, about 40 mol % to about 70 mol %, about 40 mol % to about 60 mol %, about 45 mol % to about 55 mol %, about 48 mol % to about 52 mol %, about 49 mol % to about 51 mol %, about 49.5 mol % to about 50.5 mol %, about 49.8 mol % to about 50.2 mol %, or about 50 mol %. In particular embodiments, for example when the cargo comprises a nucleic acid such as a siRNA, sihRNA or miRNA or a RNA or DNA vector, the total amount of ionizable cationic lipid(s) relative to the total amount of TCV components may be about 50 mol %.

In a preferred embodiment, a lipid-based TCV according to the present disclosure comprises DODMA at 50 mol % relative to the total amount of TCV components.

### Helper Lipid

In some embodiments, a lipid-based TCV may comprise at least one helper lipid in addition to the at least one ionizable cationic lipid. In some embodiments, the at least one helper lipid may comprise DOPE, DSPC, DOPC, DPPC, DOPG, DPPG, POPC, POPE, DOPE-mal, DPPE, DMPE, DSPE, 16-O-monomethyl PE, 16-O-dimethyl PE, 18-1-trans PE, SOPE, or any combinations thereof. In particular embodiments, the at least one helper lipid may comprise or consist of DOPE. In some cases, the at least one helper lipid to be used may be determined based on the stability of the TCV. The amount of the at least one helper lipid may be determined as appropriate.

In some embodiments, the total amount of helper lipid(s) relative to the total amount of TCV components may be about 10 mol % to about 60 mol %. In some embodiments, the total amount of helper lipid(s) relative to the total amount of TCV components may be about 10 mol % to about 50 mol %, about 10 mol % to about 40 mol %, about 20 mol % to about 40 mol %, about 25 mol % to about 35 mol %, about 28 mol % to about 32 mol %, about 29 mol % to about 31 mol %, about 29.5 mol % to about 30.5 mol %, about 29.8 mol % to about 30.2 mol %, or about 30 mol %. In particular embodiments, the total amount of helper lipid(s) relative to the total amount of TCV components may be about 30 mol %.

In some embodiments, the total amount of helper lipid(s) relative to the total amount of TCV components may be about 20 mol % to about 60 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %. In particular embodiments, the total amount of helper lipid(s) relative to the total amount of TCV components may be about mol %.

In a preferred embodiment, a lipid-based TCV according to the present disclosure comprises DOPE at 30 mol %. Such a TCV may be used, for example when the cargo comprises a nucleic acid and a protein (or a RNP).

### Phospholipid

In some embodiments, a lipid-based TCV may comprise at least one phospholipid in addition to the at least one ionizable cationic lipid. In some embodiments, the at least one phospholipid may comprise DSPC, DOPE, DPPC, DOPC, DMPC, PLPC, DAPC, PE, EPC, DLPC, DMPC, MPPC, PMPC, PSPC, DBPC, SPPC, DEPC, POPC, lysophosphatidyl choline, DSPE, DMPE, DPPE, POPE, lysophosphatidylethanolamine, or any combinations thereof. In particular embodiments, the at least one helper lipid may comprise or consist of DSPC.

In some embodiments, the amount of phospholipid(s) relative to the total amount of TCV components may be about 5 mol % to about 65 mol %, about 5 mol % to about 55 mol %, about 5 mol % to about 45 mol %, about 5 mol % to about 35 mol %, about 5 mol % to about 25 mol %, about 5 mol % to about 15 mol %, about 8 mol % to about 12 mol %, about 9 mol % to about 11 mol %, about 9.5 mol % to about 10.5 mol %, about 9.8 mol % to about 10.2 mol %, or about 10 mol %. In particular embodiments, the total amount of phospholipid(s) relative to the total amount of TCV components may be about 10 mol %.

In some embodiments, the total amount of phospholipid(s) relative to the total amount of TCV components may be about 5 mol % to about 65 mol %, about 15 mol % to about 65 mol %, about 25 mol % to about 55 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %. In particular embodiments, the total amount of phospholipid(s) relative to the total amount of TCV components may be about 40 mol %.

In a preferred embodiment, a lipid-based TCV according to the present disclosure comprises DSPC at 10 mol % relative to the total amount of TCV components. Such a TCV may be used, for example when the cargo comprises a nucleic acid molecule or nucleic acid and a protein (or a RNP complex).

### Cholesterol or Cholesterol Derivative

In some embodiments, a lipid-based TCV may comprise at least one cholesterol or cholesterol derivative in addition to the at least one ionizable cationic lipid. In some embodiments, the at least one cholesterol or cholesterol derivative may comprise cholesterol, DC-Chol, 1,4-bis(3-N-oleylamino-propyl)piperazine, ICE, or any combinations thereof. In particular embodiments, the at least one cholesterol or cholesterol derivative may comprise or consist of cholesterol.

In some embodiments, the amount of cholesterol and/or cholesterol derivative(s) relative to the total amount of TCV components may be about 20 mol % to about 60 mol %. some embodiments, the amount of cholesterol and/or cholesterol derivative(s) relative to the total amount of TCV components may be about 25 mol % to about 55 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %, or about 39%. In particular embodiments, the total amount of cholesterol and/or cholesterol derivative(s) relative to the total amount of TCV components may be about 40 mol % or about 39 mol %.

In a preferred embodiment, a lipid-based TCV according to the present disclosure comprises cholesterol at 40 mol % or 39 mol % relative to the total amount of TCV components. Such a TCV may be used, for example when the cargo comprises a nucleic acid molecule or a nucleic acid and a protein (or a RNP complex).

### PEG-Lipid

In some embodiments, a lipid-based TCV may comprise at least one PEG-lipid in addition to the at least one ionizable cationic lipid. In some embodiments, the at least one PEG-lipid may comprise PEG-DMG (e.g., (Avanti® Polar Lipids (Birmingham, AL)), PEG-phosphatidylethanolamine and phosphatidic acid, PEG-ceramide conjugates (e.g., PEG-CerC14 or PEG-CerC20), PEG-modified dialkylamines, PEG-modified 1,2-diacyloxypropan-3-amines, or any combinations thereof. In particular embodiments, the at least one PEG-lipid may comprise or consist of PEG-DMG.

In some embodiments, the amount of PEG and/or PEG-lipid(s) relative to the total amount of TCV components may be about 0.1 mol % to about 5 mol %, 0.1 mol % to about 4 mol %, 0.1 mol % to about 3 mol %, 0.1 mol % to about 2 mol %, 0.5 mol % to about 1.5 mol %, 0.8 mol % to about 1.2 mol %, 0.9 mol % to about 1.1 mol %, or about 1 mol %. In particular embodiments, the total amount of PEG-lipid(s) relative to the total amount of TCV components may be about 1 mol %.

In a preferred embodiment, a lipid-based TCV according to the present disclosure comprises PEG-DMG at 1 mol % relative to the total amount of TCV components.

In a preferred embodiment, a lipid-based TCV according to the present disclosure comprises DODMA at 20 mol %, DOPE at 30 mol %, DSPC at 10 mol %, and cholesterol at 40 mol % relative to the total amount of TCV components. Such a TCV may be used, for example when the cargo comprises a nucleic acid and a protein (or a RNP complex).

In another preferred embodiment, a lipid-based TCV according to the present disclosure comprises DODMA at 50 mol %, DSPC at 10 mol %, cholesterol at 39 mol %, PEG-DMG at 1 mol % relative to the total amount of TCV components. Such a TCV may be used, for example when the cargo comprises a nucleic acid molecule.

### TCV Size

In some embodiments, the size of TCVs may be determined by any appropriate techniques. Non-limiting examples of measurement methods include dynamic light chattering, binding assays, surface plasmon resonance (SPR), static image analysis, and dynamic image analysis. An appropriate measurement technique may be selected based on the accuracy and the approximate size range the technique is optimal for.

In some embodiments, the size of the TCV before encapsulation of the at least one cargo may be in a range of about 3 nm to about 240 nm, about 6 nm to about 160 nm, about 9 nm to about 80 nm, optionally about 10-40 nm, further optionally about 20 nm to about 40 nm or about 20-35 nm, at pH of about 4. In particular embodiments, the size of the TCV before encapsulation of the at least one cargo may be in a range of about 9 nm to about 80 nm at pH of about 4.

### Organic Solvents and Detergents

In some embodiments, one characteristic of a pharmaceutical composition is that the composition is substantially, essentially, or entirely free of ethanol, methanol, isopropanol, THF, DMSO, DMF, and ACN, and/or contains significantly lower amounts of ethanol, methanol, isopropanol, THF, DMSO, DMF, and ACN, compared to other pharmaceutical compositions comprising a similar type of TCVs.

In particular embodiments, the pharmaceutical composition may be entirely free of methanol, isopropanol, THF, DMSO, DMF, and ACN.

With regard to ethanol, in some embodiments, the pharmaceutical composition may be substantially free of ethanol, which may mean that the ethanol concentration is 5% (v/v) or below. In particular embodiments, the pharmaceutical composition may be essentially free of ethanol, which may mean that the ethanol concentration is 0.5% (v/v) or below.

In a particular embodiment, the composition may be entirely free of ethanol, methanol, isopropanol, THF, DMSO, DMF, and ACN.

In some embodiments, one characteristic of a pharmaceutical composition is that the composition is substantially, essentially, or entirely free of SDS.

In some embodiments, one characteristic of a pharmaceutical composition is that the composition is substantially, essentially, or entirely free of detergents.

In some embodiments, the composition may be substantially, essentially, or entirely free of organic solvents and detergents, and/or contains significantly lower amounts of organic solvents and detergents compared to other pharmaceutical compositions comprising a similar type of TCVs.

In some embodiments, one characteristic of a pharmaceutical composition is that the composition is substantially, essentially, or entirely free of destabilizing agents, and/or contains significantly lower amounts of destabilizing agents compared to other pharmaceutical compositions comprising a similar type of TCVs.

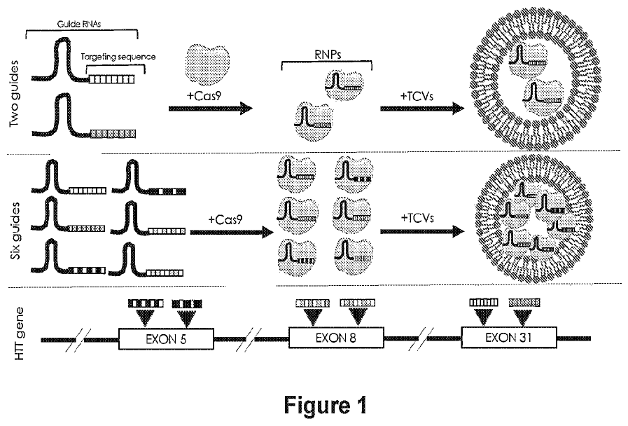

FIG. 1 depicts a schematic diagram for the inclusion of multiple guides in formation of RNP-multiple-guide mixtures, also known herein as RNP complexes, and then in subsequent encapsulation in TCVs (RNP-TCV). In FIG. 1, multiple guide RNAs are mixed together in solution prior to addition of Cas9 (or other suitable endonuclease). The guides are then incubated with Cas9 nuclease such that a mixture of RNP+multiple guides is present in solution prior to encapsulation. In other words, the mixing provides RNPs complexed with several different gRNAs. Subsequently, the RNP-multiple-guide mixture is incubated with TCVs. A significant advantage of this technology is that multiple combinations of gRNAs and RNPs are encapsulated within one vesicle, and/or within different vesicles within a single mixture. The resulting composition can be configured to be administered to a target patient and condition, including mammalian patients such as humans. There can be two or more different administrations to the animal of the therapeutic agent comprising the cationic lipid-based TCV containing the RNP. The multiple administrations can occur sequentially at different points in time, and/or can occur simultaneously at different locations on the body of the animal. The presence of multiple different guides significantly, surprisingly increases gene disruption or other alteration when administered to a target cell having a desired target nucleic acid sequence. In the current examples, multiple different guides were configured to target various, different parts within the HTT gene, in exons 5, 8 and/or 31, Each of the guides was configured to target a total target region of about 100 to 200 base pairs in length, with about 20 bp each being specifically selected to target a highly-specific region within the target sequence. In this embodiment the triplicate guides did not overlap although overlap can be utilized if desired.

While in the above set forth preferred construction, specific elements have been recited in order to adequately illustrate the principles of this invention, it will be apparent to those skilled in the art that alterations and modifications in the construction and arrangement of the system may be made without thereby departing from the spirit of said invention. Changes of form, of details of construction and materials may be made without thereby departing from the spirit of invention set forth, which shall be limited only by the scope of the appended claims. Examples are provided below to illustrate the present invention. These examples are not meant to constrain the present invention to any particular application or theory of operation.

### EXAMPLES

The following Materials and Methods were used in the examples which follow.

### Materials and Methods

### Preparation of Transfection Competent Vesicles (TCVs):

### Materials

1,2-Dioleyloxy-3-dimethylamino-propane (DODMA) was purchased from Cayman Chemical (Ann Arbor, MI). 1,2-dioleoyl-sn-glycero-3-phosphoethanolamine (DOPE), and 1,2-distearoyl-sn-glycero-3-phosphocholine (DSPC) were purchased from Avanti Polar Lipids (Alabaster, AL). Cholesterol was purchased from Sigma Aldrich (St. Louis, MO) All lipids were maintained as ethanol stocks.

### TCV and RNP Preparation

Preparation protocols for preparation of TCVs and RNPs are discussed in published Patent Cooperation Treaty patent application WO2020077007. As noted above, such publication is incorporated herein by reference for all its teachings and disclosures.

### Rapid-Mixing:

The organic phase containing lipids was mixed with the aqueous phase using the Precision Nanosystems Ignite apparatus at a final flow rate of 20 mL/min with a 1:3 organic:aqueous (v/v) ratio (Jeffs et al. (2005). Pharm Res 22(3): 362-372). Pharm Res 22(3): 362-372; Kulkari et al. (2017). Nanoscale 9(36):13600-13609; Kulkami et al. (2018). ACS Nano 12(5): 4787-4795.). The resulting suspension was dialyzed against 1000-fold volume of 25 mM sodium acetate pH 4 buffer to remove ethanol.

### Analysis of TCVs

Lipid concentrations were determined by assaying for the cholesterol content using a T-Cholesterol Assay Kit (Wako Chemicals, Mountain View, CA) and extrapolating total lipid concentration as described elsewhere (Chen et al. (2014) J Control Release 196: 106-112).

### Preparation of Ribonucleoproteins (RNPs):

### Materials

Cas9 nuclease protein was ordered from Integrated DNA Technologies (IDT) (San Jose, CA), and all guide RNAs (for targeting the HTT or luciferase gene) were ordered from Synthego (Redwood City, CA) at www.synthego.com. The targeting sequences of gRNAs used in Examples for targeting different portions of different exons (exon 2, 5, 8, or 31) of human HTT gene or the luciferase gene (an off-target gene absent in normal human or mouse cells) are shown in Table 2. Table 2 also shows the targeting sequences of gRNAs used for in vivo treatment of mice, targeting the corresponding portions of exon 31 of mouse Htt gene. Table 1 below shows the 20 nucleotide DNA sequences of human HTT gene correspond to the targeting sequences of the gRNAs for targeting different portions of HTT exons (exon 2, 5, 8, or 31).

TABLE 1
HTT DNA sequences corresponding to gRNA targeting sequences

| SEQ ID | HTT source | Internal ID | DNA sequence 5′-3′ |
|---|---|---|---|
| 1 or 410 | human exon 31 | Ex31-1 | AGATAAGCCATCAAACTGGG |
| 2 or 420 | human exon 31 | Ex31-2 | GTACAAGCCTGGCCTCACAC |
| 3 or 430 | human exon 31 | Ex31-2 | CCGCCTGCACCATGTTCCTC |
| 4 or 210 | human exon 5 | Ex5-1 | CCCCTCAGAATGGTGCCCCT |
| 5 or 220 | human exon 5 | Ex5-2 | GTTTGCTGAGCTGGCTCACC |
| 6 or 230 | human exon 5 | Ex5-3 | ACAACTTACCTGCATTTCTG |
| 7 or 310 | human exon 8 | Ex8-1 | AGGCTTACTCGTTCCTGTCG |
| 8 or 320 | human exon 8 | Ex8-2 | GCTGCTCACCCTGAGGTATT |
| 9 or 330 | human exon 8 | Ex8-3 | GTCAAGGACACAAGCCTGAA |
| 30 | human exon 2 | Ex2-1 | ATGATTCACACGGTCTTTCT |

TABLE 2
Targeting sequences of gRNAs

| SEQ ID NO: | Target | Internal ID | Targeting RNA sequence 5′-3′ |
|---|---|---|---|
| 10 | human exon 31 | Ex31-1 | AGAUAAGCCAUCAAACUGGG |
| 11 | human exon 31 | Ex31-2 | GUACAAGCCUGGCCUCACAC |
| 12 | human exon 31 | Ex31-2 | CCGCCUGCACCAUGUUCCUC |
| 13 | human exon 5 | Ex5-1 | CCCCUCAGAAUGGUGCCCCU |
| 14 | human exon 5 | Ex5-2 | GUUUGCUGAGCUGGCUCACC |
| 15 | human exon 5 | Ex5-3 | ACAACUUACCUGCAUUUCUG |
| 16 | human exon 8 | Ex8-1 | AGGCUUACUCGUUCCUGUCG |
| 17 | human exon 8 | Ex8-2 | GCUGCUCACCCUGAGGUAU |
| 18 | human exon 8 | Ex8-3 | GUCAAGGACACAAGCCUGAA |
| 19 | mouse exon 31 | m-Ex31-1 | AGAUAAGCCAUCAAACUGUG |
| 20 | mouse exon 31 | m-Ex31-2 | GCCUCACACUUGAAGAGCCA |
| 21 | mouse exon 31 | m-Ex31-3 | CCGCCUGCACCAUGUUCCUC |
| 22 | firefly | Luc | CUUCGAAAUGUCCGUUGGGU |
| | luciferase | | |
| 23 | Human exon 2 | Ex-2-1 | AUGAUUCACACGGUCUUUCU |

### RNP Formation

RNPs were formed by combining solutions of guide RNA and Cas9 protein. Briefly, a 10-μM (when multiple gRNAs were mixed, a equimolar ratio for each gRNA to achieve a total of 10 μM) gRNA solution was incubated for 5 minutes at room temperature with a 10 μM solution of Cas9 protein to form the RNP complex. The RNP solution was kept at room temperature for about 5 minutes prior to encapsulation by TCVs.

### Preparation of RNP-TCV (RNPs Encapsulated by TCVs):

8.33 μL of a 8 mM TCV solution at about pH4 (8 mM refers to the concentration of the total lipid components of TCV) and 10 μL of a 5 μM RNP solution were mixed and incubated at room temperature for 5 minutes (i.e., lipid:RNP=about at 1332.8:1 molar ratio) to encapsulate the RNPs within the TCVs.

Protocols involving formation of TCVs and RNPs and encapsulation are also described in WO2020077007.

Cell Culture and Transfection with RNP-TCV:

### Reagents

All base cell culture media and B27 neuronal supplement were purchased from Gibco (Thermo Fisher, Waltham, MA). Hank's balanced salt solution (HBSS), penicillin-streptomycin, L-glutamine, and trypsin solutions were obtained from Hyclone (Logan, UT). Primary cortical cells were plated onto tissue culture-treated plates (Fisher), coated with poly-D-lysine (Sigma, St. Louis, MO). Hygromycin B was obtained from Invitrogen (Carlsbad, CA). Recombinant ApoE4 was acquired from Peprotech (Rocky Hill, NJ).

### HEK Cells and Transfection

HEK cells were cultured in complete DMEM media containing 10% fetal bovine serum (FBS). 80,000 cells/well were plated in a 24-well plate one day prior to transfection. On the day of treatment, the media was removed completely, and fresh media containing solutions of RNP-TCV were transferred onto pre-plated cells. Cells were maintained in a humidified atmosphere at 37° C. with 5% CO2 for 72 hours prior to harvest.

### Primary Cortical Neuron Culture and Transfection

Cortices from E16.5-E17.5 embryos from YAC128 mice (which contain two copies of the normal mouse Htt gene and one copy of a full-length, mutant human HTT transgene) were dissected in ice cold HBSS, dissociated with 0.05% trypsin (Hyclone) for 10 minutes at 37° C., and triturated through a 5 mL pipette 5 times and an additional 5-7 times with a 200 μL pipette tip to achieve a single-cell suspension. Cells were pelleted by centrifugation for 5 minutes at 800 rpm, washed with HBSS, and then re-suspended in warm neurobasal media supplemented with B27, 2 mM L-glutamine (Hyclone) and 1% penicillin/streptomycin (Hyclone). Cells were plated onto poly-D lysine (PDL) (Sigma)-coated 24-well plates (Falcon) at 150,000 cells/well in Neurobasal media (Life Tech) containing B27 supplement (Gibco), 1-glutamine (Hyclone), and penicillin/streptomycin (Hyclone) and cultured for 5-7 days before transfection. Half media was changed at the time of transfection with RNP-TCV, and recombinant ApoE4 (Peprotech) was added to a final concentration of 1 μg/mL in each well. Cells were maintained in a humidified atmosphere (95%) at 37° C. with 5% CO2 for 72 hours prior to harvest.

Preparation of Neurons from iPSCs

Once iPSCs reached 60%-70% confluence, they were differentiated culturing in SLI medium (Advanced DMEM/F12 (ADF) supplemented with 2 mM Glutamax, 2% B27 without vitamin A (all Life Technologies)), 10 μM SB431542 (Stem Cell Technologies), 1 μM LDN 193189 (Miltenyi Biotec) and 1.5 μM IWR1 (Tocris)), and media was changed daily for 8 days, with a 1:2 cell passage occurring on day 4. On day 8, cells were passaged again and transferred into LI medium (ADF supplemented with 2 mM Glutamax, 2% B27 without vitamin A, 0.2 μM LDN 193189 and 1.5 μM IWR1) or LIA (LI+20 ng/ml Activin A (Peprotech); modified from Telezhkin et. al., 2016 (PMID:26718628)) until day 16. On day 16, cultures were re-plated in SCM1 medium (ADF supplemented with 2 mM Glutamax, 2% B27 supplement, 2 μM PD 0332991 (Tocris, USA), a CDK4/6 inhibitor, 10 μM DAPT (Tocris, USA), 10 ng/ml BDNF, (Peprotech), 10 μM Forskolin, 3 μM CHIR 99021, 300 μM γ-amino butyric acid (GABA, all Tocris), supplemented with CaCl2) to final concentration of 1.8 mM) and 200 M ascorbic acid (both Sigma-Aldrich)). Cells were plated onto 100 μg/ml poly-D-lysine (Sigma-Aldrich)-/hESC-qualified Matrigel-coated 24-well plate, at a concentration of 250,000 cells per well. A 50% medium change was performed every 2-3 days. Cells were treated with RNP-TCV on day 18, during regular half media changes.

Transfection of iPSC-Derived Neurons

50% media change was performed on day 18, and ApoE4 (Peprotech) was added to a final concentration of 1 μg/mL in each well. Cultures were treated with either 83 nM of RNP-TCV or left untreated as a control. Cells were maintained in a humidified atmosphere at 37° C. with 5% CO2 for 72 hours prior to collection for RNA quantification.

### In Vivo Mouse Brain Injections:

### Protocol 1—Single Site Vs Two Sites

Wild-type C57BL/6 mice, approximately 2-3 months of age, were used in this study. 2 μL of a RNP-TCV solution targeting Htt exon 31 (the gRNAs contained is a mixture of three gRNAs with targeting sequences of SEQ ID NOS: 19, 20, and 21) or, as a control, Luc (gRNA having the targeting sequence of SEQ ID NO: 22) as prepared above was injected bilaterally into the striatum, either as one bolus (single location) or split as two 1 μL-injections. For the single location, the injection took place 0.5 mm anterior to Bregma, medial/lateral (ML)+/−1.8 mm left or right from center, and 3.5 mm depth, relative to brain surface. For the double injection group, the injection was split into two 1 μL, at 0.5 mm and 1.0 mm anterior of Bregma, with the same ML and depth coordinates. In all cases, the infusion rate was 0.5 μL/min.

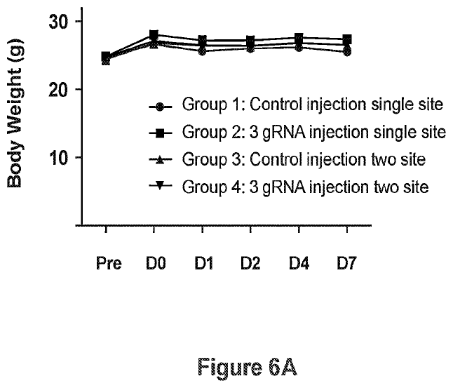

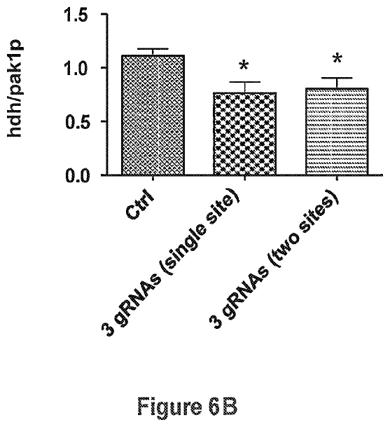

Mice were monitored for 7 days post-injection for general health and body weight was recorded daily. All survived, and no weight loss was reported following treatment, suggesting healthy mice. See FIG. 6A. 7 days post-injection, mice were sacrificed, perfused with PBS, and micro-dissected for brain regions. The striatum was removed and snap frozen for RNA processing and qPCR quantification.

### Protocol 2—Single Dose Vs Multiple Doses

Wild-type C57BL/6 mice, approximately 2-3 months of age, received 1, 2, or 3 treatments, two weeks apart, each with 2 μL of a RNP-TCV solution (containing about 5.46 μmol RNP in TCVs corresponding to about 7.2 nmol lipids) as prepared above. The gRNAs contained were a mixture of three gRNAs against mouse HTT exon 31 with targeting sequences of SEQ ID NOS: 19, 20, and 21. For each treatment with the 2 μL, a 1 μL dose was directly injected into each hemisphere of the brain, similarly to the double injections of Protocol 1 above. All mice survived and no observable toxic side effects were reported after administration.

RNA Quantification by qPCR:
Quantification for Cells from Culture

Cells were washed once in sterile PBS prior to being scraped off the plate in 600 μL lysis buffer containing 1% 2-mercaptoethanol and snap frozen on dry ice prior to storage at −80° C. Total RNA was extracted using the PureLink RNA mini kit (Invitrogen) according to the manufacturer's instructions. Reverse transcription of all samples was carried out using the Superscript VILO kit (Invitrogen) according to the manufacturer's instructions, using 250 ng of total RNA as input for cDNA synthesis and ng diluted RNA for the quantitative PCR (qPCR) reaction. Quantification of mouse Htt mRNA levels was accomplished using the standard curve method, with amplification of target mRNA and control genes in separate wells, performed using FastSybr (Applied Biosystems) and conducted on a Step-One ABI System (Applied Biosystems). Each sample was run in duplicate. The relative amount of mRNA in each well was calculated as the ratio between Htt mRNA and a control gene, Csnk2a2 or Pak1p. Quantification of human HTT mRNA levels was accomplished using the standard curve method, with amplification of target mRNA and control genes in separate wells, performed using FastSybr (Applied Biosystems) and conducted on a Step-One ABI System (Applied Biosystems). Each sample was run in duplicate. The relative amount of mRNA in each well was calculated as the ratio between HTT mRNA and a control gene, RPLO or PGK1. Values are presented as relative to control gene, N=3 wells per condition.

### Quantification for Tissue Samples

Snap frozen brain tissue samples were dissociated using the FastPrep-24 (MP BioMedical) in tubes containing lysing matrix D and lysis buffer from the RNA mini kit (Invitrogen) and processed as above. Quantification of mouse Htt mRNA levels was accomplished using the standard curve method, with amplification of target mRNA and control genes in separate wells, performed using FastSybr (Applied Biosystems) and conducted on a Step-One ABI System (Applied Biosystems). Each sample was run in duplicate. The relative amount of mRNA in each well was calculated as the ratio between Htt mRNA and a control gene, Pak1p.

### Statistical Analyses:

Unless otherwise specified, statistical comparisons were performed as a one-way analysis of variance (ANOVA) with Bonferroni post-hoc analysis to compare individual means to control-treated cells and correct for multiple comparisons (Prism 6, Graphpad Software Inc.). A Student's t-test was used to compare individual means in the case of only two groups. A p-value less than 0.05 (marked with *) was considered significant.

### Results

### Example 1: Targeting Exon 31 but not Exon 2 Results in Successful Gene Editing of Human HTT

First, HEK cells were transfected with RNP-TCV targeting either exon 2 (with a gRNA having the targeting sequence of SEQ ID NO: 23, corresponding to SEQ ID NO: 30) or exon 31 (with a mixture of 3 gRNAs having the targeting sequences of SEQ ID NOS: 10, 11, and 12, corresponding to SEQ ID NOS: 1 (or 410), 2 (or 420), and 3 (or 430), respectively) of human HTT or, as a negative control, the firefly luciferase (Luc) gene (with the targeting sequence of SEQ ID NO: 22), at an RNP concentration of 50 nM. Changes in the transcript levels of HTT were analyzed.

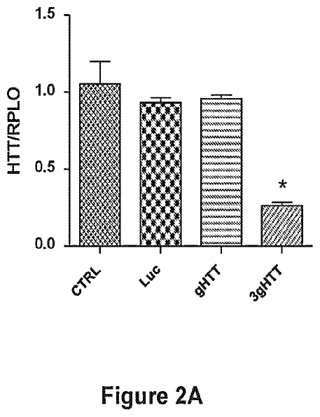

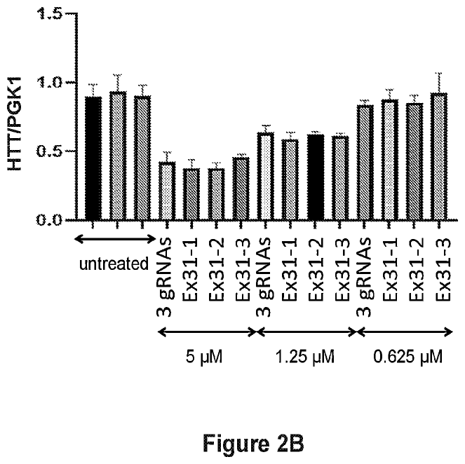

FIG. 2A provides exemplary transcript levels of HTT relative to the housekeeping gene RPLO in each group. As shown in FIG. 2A, while targeting of exon 2 did not results in any significant reduction in the HTT transcripts (similar levels as when the Luc-targeting gRNA was used), targeting exon 31 using the three different gRNAs achieved statistically significant levels of reduction in the HTT transcripts, about 75% reduction.

To test the effects by individual gRNAs of the three gRNAs targeting exon 31, HEK cells were transfected with RNP-TCV targeting exon 31 of human HTT using one of the three gRNAs (the targeting sequence of either SEQ ID NO: 10, 11, or 12, corresponding to SEQ ID NO: 1 (or 410), 2 (or 420), or 3 (or 430), respectively) or all three gRNAs at an RNP concentration of 5, 1.25, or 0.625 μM. FIG. 2B provides exemplary transcript levels of HTT relative to the housekeeping gene PGK1 in each group. One-way ANOVA: p=<0.0001. Tukey tests (applied to any pairwise comparisons between the following groups): untreated vs 5 μM, p<0.0001; untreated vs 1.25 μM, p<0.001; and untreated vs 0.625 μM, N.S. As shown in FIG. 2B, all three gRNAs successfully achieved reduction in the HTT transcripts in a dose-dependent manner.

### Example 2: The Set of Three gRNAs Against Human HTT Exon 31 Works Against the Huntingtin Gene of Both Mouse and Human in Primary Neurons

Primary mouse cortical neurons from FVB.YAC128 mice, which contain the normal mouse Htt gene and a full-length mutant human HTT transgene, were transfected with RNP-TCV targeting human HTT exon 31 (with a mixture of 3 gRNAs having the targeting sequences of SEQ ID NOS: 10, 11, and 12, corresponding to SEQ ID NOS: 1 (or 410), 2 (or 420), and 3 (or 430), respectively) or, as a negative control, the Luc gene (with a gRNA having the targeting sequence of SEQ ID NO: 22), at an RNP concentration of 50 nM. Changes in the transcript levels of Htt and HTT were analyzed.

The targeting sequence of gRNA “Ex31-1” has 1 mismatch at the 2^{nd} nucleotide counting from the PAM site, relative to the corresponding portion of mouse Htt exon 31. The gRNA “Ex31-2” should not be functional for mouse Htt exon 31 due to a sequence difference within the PAM site between human and mouse genes. The targeting sequence of gRNA “Ex31-3” against human HTT exon 31 has zero mismatches relative to the corresponding portion of mouse Htt exon 31.

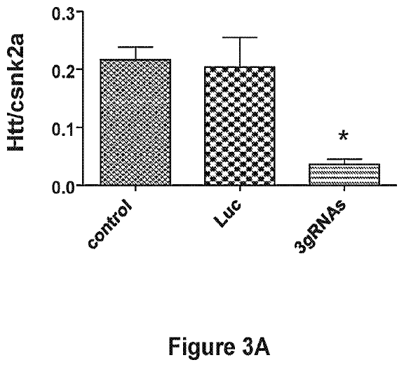

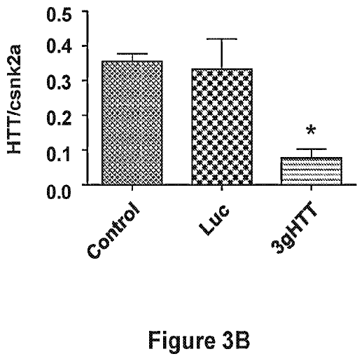

FIGS. 3A and 3B provide exemplary transcript levels of mouse huntingtin (Htt) (A) and human huntingtin (HTT) (B) relative to the housekeeping gene csnk2a in each group As shown in FIG. 3B, the set of three gRNAs successfully achieved statistically significant reduction (by about 80%) in the HTT transcripts in primary neurons. Interestingly, as shown in FIG. 3A, the same set of three gRNAs also achieved statistically significant reduction (by about 75%) in the Htt transcripts. Based on these results, the fact that one of the three gRNAs comprises one mismatch (at the 2^{nd} nucleotide from the PAM site) to the Htt gene does not seem to have a significant impact on the gene editing efficacy.

### Example 3: The Set of Three gRNAs Against Human HTT Exon 31 Works in Human iPSC-Derived Neurons

Human iPSC-derived neurons were transfected with RNP-TCV targeting human HTT exon 31 (with a mixture of 3 gRNAs having the targeting sequences of SEQ ID NOS: 10, 11, and 12, corresponding to SEQ ID NOS: 1 (or 410), 2 (or 420), and 3 (or 430), respectively) or, as a negative control, the Luc gene (with a gRNA having the targeting sequence of SEQ ID NO: 22), at an RNP concentration of 83 nM. Changes in the transcript levels of Htt and HTT were analyzed.

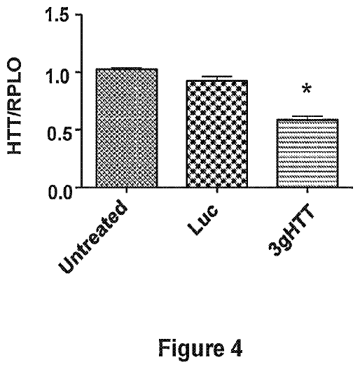

FIG. 4 provides exemplary transcript levels of HTT relative to the housekeeping gene RPLO in each group. As shown in FIG. 4, the set of three gRNAs achieved statistically significant reduction (by about 40%) in the HTT transcripts.

### Example 4: The Set of Three gRNAs Against Human HTT Exon 5, 8, or 31 or Nine gRNAs Against Human HTT Exon 5, 8, and 31 Works in HEK293 Cells

HEK293 cells were transfected with RNP-TCV targeting: HTT exon 31 (with a mixture of 3 gRNAs having the targeting sequences of SEQ ID NOS: 10, 11, and 12, corresponding to SEQ ID NOS: 1 (or 410), 2 (or 420), and 3 (or 430), respectively); HTT exon 5 (with a mixture of 3 gRNAs having the targeting sequences of SEQ ID NOS: 13, 14, and 15, corresponding to SEQ ID NOS: 4 (or 210), 5 (or 220), and 6 (or 230), respectively); HTT exon 8 (with a mixture of 3 gRNAs having the targeting sequences of SEQ ID NOS: 16, 17, and 18, corresponding to SEQ ID NOS: 7 (or 310), 8 (or 320), and 9 (or 330), respectively); HTT exons 5, 8, and 31 (with a mixture of the 9 gRNAs having the targeting sequences of SEQ ID NOS: 10, 11, 12, 13, 14, 15, 16, 17, and 18, corresponding to SEQ ID NOS: 1 (or 410), 2 (or 420), 3 (or 430), 4 (or 210), 5 (or 220), and 6 (or 230), 7 (or 310), 8 (or 320), and 9 (or 330), respectively); or, as a negative control, the Luc gene (with the targeting sequence of SEQ ID NO: 22), at an RNP concentration of 50 nM. Changes in the transcript levels of Htt and HTT were analyzed.

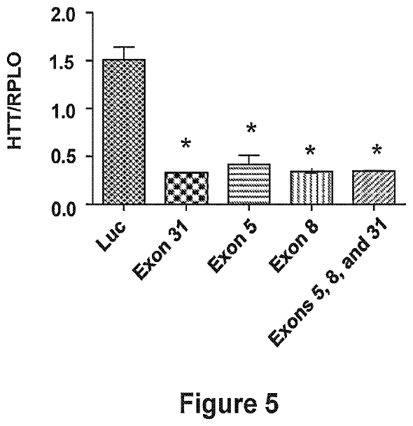

FIG. 5 provides exemplary transcript levels of HTT relative to the housekeeping gene RPLO in each group. As shown in FIG. 5, all sets of three gRNAs tested achieved statistically significant reduction (by about 75-80%) in the HTT transcripts.

### Example 5: Direct Injection of the Set of Three gRNAs Against Mouse Htt Exon 31 into Mouse Brain at a Single or Two Sites Successfully Edits Htt

Mice received a direct injection of a RNP-TCV solution targeting mouse Htt exon 31 (the gRNAs contained is a mixture of three gRNAs with targeting sequences of SEQ ID NOS: 19, 20, and 21) or, as a control, Luc (a gRNA having the targeting sequence of SEQ ID NO: 22) into the striatum at a single or two sites, according to the method of “Protocol 1” described above. All mice survived the injection(s). Changes in the body weight were recorded. The striatum 7 days post-injection was analyzed for Htt transcript levels.

FIG. 6A provides exemplary changes in the body weight. As shown in FIG. 6A, no notable differences in the body weight changes were observed, suggesting healthy mice. FIG. 6B provides exemplary transcript levels of Htt relative to the housekeeping gene pak1p in each group. As shown in FIG. 6B, the three gRNAs administered via direct injection into the adult mouse brain, regardless of whether the administration was at a single site or two different sites, achieved statistically significant reduction (by about 25-30%) in the Htt transcripts.

### Example 6: Comparison of a Single or Multiple Direct Injections of the Set of Three gRNAs Against Mouse Htt Exon 31 into Mouse Brain

Mice received one, two, or three direct injections, two weeks apart, of a RNP-TCV solution targeting mouse Htt exon 31 (the gRNAs contained is a mixture of three gRNAs with targeting sequences of SEQ ID NOS: 19, 20, and 21) or, as a control, Luc (a gRNA having the targeting sequence of SEQ ID NO: 22) into each striatum, according to the method of “Protocol 2” described above. All mice survived the injection(s). Changes in the body weight were recorded. No signs of toxicity have been observed.

### Definitions

All references cited herein, including patent documents and non-patent documents, are hereby incorporated by reference in their entirety. Nothing herein is to be construed as an admission that the present invention is not entitled to antedate such disclosure by virtue of prior invention.

All terms used herein are used in accordance with their ordinary meanings as commonly understood by one of ordinary skill in the art to which this disclosure belongs, unless the context or definition clearly indicates otherwise.

Singular forms, including in the claims, such as “a,” “an,” and “the” as used herein and in the appended claims include the plural reference, unless expressly stated, or the context clearly indicates, otherwise. Thus, the reference to “a cell” refers to one or more cells and equivalents thereof known to those skilled in the art, and so forth.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

In the specification above and in the appended claims, all transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

Unless otherwise stated, terms such as “substantially”, “essentially”, and “about” that modify another term describing a condition or relationship characteristic etc of a feature or features of an embodiment indicate that the condition or characteristic is defined to be within tolerances that are acceptable for operation of the embodiment for an application for which it is intended. For example, a composition comprising essentially of certain ingredient(s) necessarily includes the listed ingredient(s) and is open to unlisted ingredients that do not materially affect the intended characteristic(s) according to the invention.

The term “cargo” or “cargo molecule” as used herein is one or more materials carried by and/or encapsulated by/in a TCV according to the present disclosure. In some embodiments, the combination of materials carried by a TCV may be collectively referred to as a “cargo”. In some embodiments, a TCV may carry an endonuclease protein such as Cas9 as a cargo. In some embodiments, a TCV may carry one or more gRNAs as a cargo. In some embodiments, a TCV may carry template DNA as a cargo. In some embodiments, a TCV may carry a combination of an endonuclease protein and a guide RNA as a cargo, optionally in a form of a RNP formed by the gRNA and the endonuclease. In some embodiments, a TCV may carry a mixture of different RNPs comprising different gRNAs. In some embodiments, a TCV may carry a polynucleotide encoding an endonuclease protein such as Cas9 as a cargo. In some embodiments, a TCV may carry a polynucleotide encoding one or more gRNAs as a cargo. In some embodiments, a TCV may carry (i) a polynucleotide encoding an endonuclease protein such as Cas9 and (ii) a polynucleotide encoding one or more gRNAs as a cargo. In some embodiments, a TCV may carry (i) a polynucleotide encoding an endonuclease protein such as Cas9 and (ii) one or more gRNAs as a cargo. In some embodiments, a TCV may carry (i) an endonuclease protein such as Cas9 and (ii) a polynucleotide encoding one or more gRNAs as a cargo. In some embodiments, a TCV may carry a polynucleotide encoding both an endonuclease protein such as Cas9 and one or more gRNAs as a cargo. Optionally, any of such TCVs may further carry a template DNA and/or a polynucleotide encoding a template RNA.

The term “cholesterol derivative” as used herein, in its broadest sense, encompasses any derivatives of cholesterol. Non-limiting examples of cholesterol derivatives include: DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or imidazole cholesterol ester (ICE) (US20210220273A1).

Clustered regularly interspaced short palindromic repeats (CRISPR)/CRISPR-associated (Cas) systems are a class of genome-editing tools that target desired genomic sites in mammalian cells. A CRISPR/Cas system involves at least one Cas endonuclease and a gRNA. Typically, the Cas endonuclease may recognize a protospacer adjacent motif (PAM) sequence specific to the Cas endonuclease (with certain Cas types, a protospacer flanking site (PFS) instead of a PAM) in the target gene (sense or antisense strand) and if the gRNA is able to hybridize with a target sequence in the target gene proximate to the PAM or PFS site, the Cas endonuclease may mediate cleavage of the target gene at about 2-6 nucleotides upstream of the PAM site or a particular position relative to the PAM or PFS site. Class 2 Type II CRISPR/Cas systems use Cas9 endonuclease, and for example the PAM sequence for Streptococcus pyogenes Cas9 (SpCas9) is 5′-NGG-3′. SpCas9 is targeted to a genomic site by complexing with a guide RNA that hybridizes to an approximately 17-24-nucleotide DNA sequence immediately preceding an 5′-NGG-3′ motif (where “N” can be any nucleotide) recognized by SpCas9 (e.g., a (N)17-24NGG target DNA sequence; “N” represents any nucleotide). This results in a double-strand break (DSB) between the third and fourth nucleotides upstream of the NGG motif. The DSB instigates either non-homologous end-joining (NHEJ), which typically leads to the introduction of one or more nucleotide insertions or deletions resulting in frameshift mutations that knock out gene alleles, or homology-directed repair (HDR), which can be exploited with the use of an exogenously introduced double-strand or single-strand template DNA to knock in or correct a mutation in the genome.

Any appropriate Cas endonucleases may mediate CRISPR-mediated gene editing. In some embodiments, a Cas endonuclease (as a protein) or a Cas endonuclease-encoding polynucleotide (e.g., DNA or RNA) may be used. In some embodiments, the Cas endonuclease may be a Cas of Class 1 CRISPR/Cas system (Type I, III, or IV) or Class 2 CRISPR/Cas system (Type II, V, or VI). In some embodiments, the Cas endonuclease may be Cas 9, Cas3, Cas8a2, Cas8b, Cas8c, Cas10, Cas11, Cas12, Cas12a or Cpf1, Cas13, Cas13a, C2c1, C2c3, or C2c2. In some embodiments, the Cas endonuclease may be a class 2 Cas endonuclease. In some embodiments, the Cas endonuclease may be a type II, V, or VI Cas endonuclease. In certain embodiments, the Cas endonuclease is Cas9. In certain embodiments, the Cas9 may be Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, eSpCas9, HypaCas9, FokI-Fused dCas9, xCas9, SpRY (variant of SpCas9), or SpG (variant of SpCas9), which are collectively referred to as Cas9 herein. Cas endonucleases of different bacterial origins often recognize different PAM sequences and/or different cleavage accuracy or specificity. In some cases, the type of Cas endonuclease to use may be selected based on the presence or absence or a certain PAM sequence in the target gene.

In some embodiments, the Cas endonuclease may be a wild-type (WT) SpCas9. WT SpCas9 may comprise the amino acid sequence of SEQ ID NO: 600. In some embodiments, the Cas endonuclease may be a variant SpCas9. A variant SpCas9 may comprise one or more amino acid modifications relative to SEQ ID NO: 600.

In embodiments, the Cas9 variant may comprise a substitution at position 80 of SEQ ID NO: 600, e.g., includes a leucine at position 80 of SEQ ID NO: 600 (i.e., comprises, e.g., consists of, SEQ ID NO: 600 with a C80L substitution). In embodiments, the Cas9 variant may comprise a substitution at position 574 of SEQ ID NO: 600, e.g., includes a glutamic acid at position 574 of SEQ ID NO: 600 (i.e., comprises, e.g., consists of, SEQ ID NO: 600 with a C574E substitution). In embodiments, the Cas9 variant may comprise a substitution at position 80 and a substitution at position 574 of SEQ ID NO: 600, e.g., includes a leucine at position 80 of SEQ ID NO: 600, and a glutamic acid at position 574 of SEQ ID NO: 600 (i.e., comprises, e.g., consists of, SEQ ID NO: 600 with a C80L substitution and a C574E substitution). Without being bound by theory, it is believed that such substitutions improve the solution properties of Cas9.

In embodiments, the Cas9 variant may comprise a substitution at position 147 of SEQ ID NO: 600, e.g., includes a tyrosine at position 147 of SEQ ID NO: 600 (i.e., comprises, e.g., consists of, SEQ ID NO: 600 with a D147Y substitution). In embodiments, the Cas9 variant may comprise a substitution at position 411 of SEQ ID NO: 600, e.g., includes a threonine at position 411 of SEQ ID NO: 600 (i.e., comprises, e.g., consists of, SEQ ID NO: 600 with a P411T substitution). In embodiments, the Cas9 variant may comprise a substitution at position 147 and a substitution at position 411 of SEQ ID NO: 600, e.g., includes a tyrosine at position 147 of SEQ ID NO: 600, and a threonine at position 411 of SEQ ID NO: 600 (i.e., comprises, e.g., consists of, SEQ ID NO: 600 with a D147Y substitution and a P411T substitution). Without being bound by theory, it is believed that such substitutions improve the targeting efficiency of Cas9, e.g., in yeast.

In embodiments, the Cas9 variant may comprise a substitution at position 1135 of SEQ ID NO: 600, e.g., includes a glutamic acid at position 1135 of SEQ ID NO: 600 (i.e., comprises, e.g., consists of, SEQ ID NO: 600 with a D1135E substitution). Without being bound by theory, it is believed that such substitutions improve the selectivity of Cas9 for the NGG PAM sequence versus the NAG PAM sequence.

In embodiments, Cas9 may be a variant SpCas9 that includes one or more substitutions relative to SEQ ID NO: 600 that introduce an uncharged or nonpolar amino acid, e.g., alanine, at certain positions. In embodiments, Cas9 may be a variant SpCas9, which, relative to SEQ ID NO: 600, includes a substitution at position 497, a substitution at position 661, a substitution at position 695 and/or a substitution at position 926 of SEQ ID NO: 600, for example a substitution to alanine at position 497, position 661, position 695 and/or position 926 of SEQ ID NO: 600. In embodiments, Cas9 has a substitution only at position 497, position 661, position 695, and position 926 of SEQ ID NO: 600, relative to SEQ ID NO: 600, e.g., where each substitution is to an uncharged amino acid, for example, alanine. Without being bound by theory, it is believed that such substitutions reduce the cutting by Cas9 at off-target sites.

It will be understood that the substitutions described herein to Cas9 may be combined, and may be combined with any of the fusions or other modifications described herein. In certain embodiments, Cas9 may comprise or consist of any of the amino acid sequences of SEQ ID NOS: 600-611. In particular embodiments, Cas9 may comprise or consist of the amino acid sequence of SEQ ID NO: 600. Some of the Cas endonucleases and variants thereof that may be used in the present invention further include but are not limited to those described in, e.g., WO2017115268A1 or SEQ ID NO: 1-612 of U.S. Ser. No. 11/118,177.

The term “destabilizing agent” as used herein encompasses any agents that destabilizes the cargo of a TCV according to the present disclosure. In some embodiments, a destabilizing agent may destabilize or degrade a nucleic acid cargo such as a gRNA, a protein cargo such as a Cas endonuclease, and/or a RNP. Exemplary destabilizing agents include but are not limited to: organic solvents such as ethanol and detergents such as sodium dodecyl sulfate (SDS). In some embodiments, a TCV or a composition according to the present disclosure may be substantially free of destabilizing agents. In some embodiments, a TCV or a composition according to the present disclosure may be substantially free of organic solvents and detergents. In some embodiments, a TCV or a composition according to the present disclosure may be substantially free of organic solvents. In some embodiments, a TCV or a composition according to the present disclosure may be substantially free of detergents. In some embodiments, such a TCV or a composition according to the present disclosure may be substantially, essentially, or entirely free of ethanol, methanol, isopropanol, tetrahydrofuran (THF), dimethyl sulfoxide (DMSO), dimethyl formamide (DMF), and acetonitrile (ACN). In some embodiments, a TCV or a composition according to the present disclosure may have a final ethanol concentration of 5% (v/v) or below, preferably 0.5% (v/v).

“Guide RNA” or “gRNA”, as used herein in relation to the CRISPR/Cas gene editing (also referred to as “CRISPR-mediated gene editing), refers to a RNA fragment (e.g., single guide RNA (“sgRNA”)) or a hybrid of two RNA fragments (e.g., dual guide RNA (“dgRNA”)) that binds to a target DNA sequence and guide a Cas endonuclease protein to the specific site of a DNA (e.g., in a genome) to allow for Cas-mediated cleavage of a DNA molecule. In some embodiments, gRNA may be dgRNA comprising: (I) a crispr RNA (crRNA), which comprises (i) a targeting sequence of about 15-75 nucleotides that is complementary to (or comprising some mismatches relative to) the target DNA sequence and (ii) a crRNA flagpole sequence; and (II) a trans-activating crispr RNA (tracrRNA), which comprises (i) a tracrRNA flagpole sequence and (ii) tracrRNA endonuclease binding domain, which serves as a binding scaffold for the Cas endonuclease, wherein the crRNA and tracrRNA hybridize with each other via the flagpole sequences. In some embodiments, a gRNA may be sgRNA comprising (I) a crRNA sequence linked to (II) a tracrRNA sequence as a single polynucleotide.

In some embodiments, the dgRNA and sgRNA may have the following formats:
dgRNA
crRNA (polynucleotide 1 having a crRNA sequence):
[targeting sequence]-[crRNA flagpole sequence]-[(optional) crRNA first flagpole extension]-[(optional) crRNA second flagpole extension]
* the sequence of [crRNA flagpole sequence]-[(optional) crRNA first flagpole extension]-[(optional) crRNA second flagpole extension] may be referred to herein as “crRNA backbone sequence”.
tracrRNA (polynucleotide 2 having a tracrRNA):
[(optional) tracrRNA first extension]-[tracrRNA flagpole sequence]-[tracrRNA endonuclease binding domain]
sgRNA (having a crRNA sequence linked to a tracrRNA sequence)
[targeting sequence]-[crRNA flagpole sequence]-[(optional) crRNA first flagpole extension]-[(optional) linker]-[(optional) tracrRNA first extension]-[tracrRNA flagpole sequence]-[tracrRNA endonuclease binding domain]
* the sequence of [crRNA flagpole sequence]-[(optional) crRNA first flagpole extension]-[(optional) linker]-[(optional) tracrRNA first extension]-[tracrRNA flagpole sequence]-[tracrRNA endonuclease binding domain] may be referred to herein as “sgRNA backbone sequence”.

In some embodiments, the crRNA flagpole sequence may comprise SEQ ID NO: 101 or 102. In some embodiments, the optional crRNA first flagpole extension may comprise SEQ ID NO: 103. In some embodiments, the optional crRNA second flagpole extension may comprise SEQ ID NO: 104. In some embodiments, the optional tracrRNA first extension may comprise SEQ ID NO: 105. In some embodiments, the tracrRNA flagpole sequence may comprise SEQ ID NO: 106 or 107. In some embodiments, the tracrRNA endonuclease binding domain may comprise SEQ ID NO: 108. In some embodiments, the tracrRNA endonuclease binding domain may further comprise or may be followed by one or more uracil based, e.g., 5′-U-3′, 5′-UU-3′, 5′-UUU-3′, 5′-UUUU-3′, 5′-UUUUU-3′, 5′-UUUUUU-3′, 5′-UUUUUUU-3′, or 5′-UUUUUUUU-3′.

In certain embodiments, the crRNA flagpole sequence may comprise SEQ ID NO: 101 and the tracrRNA flagpole sequence may comprise SEQ ID NO: 106. In certain embodiments, the crRNA flagpole sequence may comprise SEQ ID NO: 102 and the tracrRNA flagpole sequence may comprise SEQ ID NO: 107. In some embodiments, the optional linker which links a crRNA and tracrRNA in a sgRNA may comprise or consist of SEQ ID NO: 109.

In some embodiments, a sgRNA may comprise a sgRNA backbone sequence (the sequence which is placed 3′ to a targeting sequence in a sgRNA) of any of SEQ ID NOS: 111-114. In certain embodiments, the sgRNA backbone sequence may be followed by one or more uracils. In particular embodiments, the sgRNA backbone sequence may be followed by 1-10 uracils, such as 3 uracils, 4 uracils, 5 uracils, 6 uracils, 7 uracils, or 8 uracils.

In some embodiments, a dgRNA may comprise (I) a crRNA sequence comprising a crRNA backbone sequence (the sequence which is placed 3′ to a targeting sequence in a crRNA) comprising SEQ ID NO: 115 and (II) a tracrRNA sequence comprising SEQ ID NO: 116. In some embodiments, a dgRNA may comprise (I) a crRNA sequence comprising a sgRNA backbone sequence (the sequence which is placed 3′ to a targeting sequence in a crRNA) comprising SEQ ID NO: 117 and (II) a tracrRNA sequence comprising SEQ ID NO: 118.

When Cas9 is used, in some embodiments, the targeting sequence may comprise a GC content in the range of 40-80%, and in some embodiments, and the targeting sequence may have a length of 17-24 nucleotides.

In some embodiments, a gRNA according to the present disclosure may comprise one or more modifications. In some embodiments, the modification may be selected from the group consisting of: 2′-O-C1-4alkyl such as 2′-O-methyl (2′-OMe), 2′-deoxy (2′-H), 2′-O-C1-3alkyl-O-C1-3alkyl such as 2′-methoxyethyl (2′-MOE), 2′-fluoro (2′-F), 2′-amino (2′-NH2), 2′-arabinosyl (2′-arabino) nucleotide, 2′-F-arabinosyl (2′-F-arabino) nucleotide, 2′-locked nucleic acid (LNA) nucleotide, 2′-unlocked nucleic acid (ULNA) nucleotide, a sugar in 1 form (1-sugar), and 4′-thioribosyl nucleotide. In some embodiments, the modification is an internucleotide linkage modification selected from the group consisting of: phosphorothioate, phosphonocarboxylate, thiophosphonocarboxylate, alkylphosphonate, and phosphorodithioate. In some embodiments, the modification is selected from the group consisting of: 2-thiouracil (2-thioU), 2-thiocytosine (2-thioC), 4-thiouracil (4-thioU), 6-thioguanine (6-thioG), 2-aminoadenine (2-aminoA), 2-aminopurine, pseudouracil, hypoxanthine, 7-deazaguanine, 7-deaza-8-azaguanine, 7-deazaadenine, 7-deaza-8-azaadenine, 5-methylcytosine (5-methylC), 5-methyluracil (5-methylU), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (5-allylU), 5-allylcytosine (5-allylC), 5-aminoallyluracil (5-aminoallylU), 5-aminoallyl-cytosine (5-aminoallylC), an abasic nucleotide, Z base, P base, Unstructured Nucleic Acid (UNA), isoguanine (isoG), isocytosine (isoC), and 5-methyl-2-pyrimidine. In particular embodiments, a gRNA may comprise (i-1) 2-O-methylation further optionally at first three and last three bases and/or (i-2) one or more 3′ phosphorothioate bonds, further optionally between first three and last two bases.

A targeting sequence of a gRNA may be any appropriate length. The most frequently used targeting sequence length is 20 nt. In some embodiments, a gRNA longer than 20 nt may be used. For example, Ran et al. demonstrated that longer gRNAs are commonly cleaved to a shorter length so that the targeting sequence is e.g., 20 nt and thus the complementarity in the segment in excess of 20 nt may not be important, i.e., may or may not be complementary to a target sequence (Ran et al., Cell. 2013 Sep. 12; 154(6):1380-9.). In some embodiments, a gRNA shorter than 20 nt may be used. For example, Fu et al. demonstrated that truncated (i.e., <20 nt) gRNAs, which is as short as 17, 18, or 19 nt, may also target the same target as a corresponding 20 nt-long gRNA and perhaps even may have decreased off-target effects (Fu et al. Nat Biotechnol. 2014 March; 32(3): 279-284.).

A targeting sequence of a gRNA may or may not comprise a mismatch relative to the target sequence. In some cases, a mismatch at a particular position may reduce gRNA specificity to the target sequence. For example, in the context of SpCas9, Cong et al demonstrated that complementarity at up to 11 nt from the 3′-end of a targeting sequence is more important than that at a more upstream region (Cong et al., Science. 2013 Feb. 15; 339(6121): 819-823.). Again in the context of SpCas9, Zheng et al demonstrated that the core sequence which is from the 4th to the 7th nt from the 3′-end is more sensitive to target mismatch compared to the rest of the targeting sequence (Zheng et al., Sci Rep. 2017 Jan. 18; 7:40638.). Therefore, in some embodiments, a gRNA targeting sequence may comprise a mismatch relative to its target sequence outside of such a core sequence.

The term “helper lipid” or “structural lipid” as used herein refers to a type of lipid that may be comprised in a TCV in addition to an ionizable cationic lipid. In some embodiments, a helper lipid may be a non-cationic lipid and may be neutral, zwitterionic, or anionic lipid. In some embodiments, a helper lipid may be a lipid that carries a net negative charge at a selected pH, such as physiological pH. Without wishing to be bound by theory, helper lipids in TCVs in general are used to provide particle stability and/or biocompatibility and/or to enhance cargo delivery efficiency. Non-limiting helper lipids include, but are not limited to dioleoylphosphatidylethanolamine (DOPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof. In some embodiments, a helper lipid is dioleoylphosphatidylethanolamine (DOPE).

“Huntington's disease” or “HD” is a genetically inherited, autosomal dominant neurodegenerative disorder characterized by progressive cognitive deficits, psychiatric disruption, and loss of motor coordination (Sandou and Humbert, Neuron. 2016 Mar. 2; 89(5):91026.). Cognitive symptoms can be detected up to a decade before diagnosis. The cognitive deficits include cognitive slowing and decreases in both attention and mental flexibility. Psychiatric symptoms and/or emotional deficits are also observed early in HD patients. HD patients are frequently depressed and show signs of apathy, irritability, impulsivity, and social disinhibition. Motor symptoms include choreiform movements with gait disturbances that tend to appear early in the course of the disease and motor impairments such as bradykinesia and rigidity that are observed in later stage patients. The neuropathology of HD is characterized by the dysfunction and death of specific neurons within the brain. Neurons of the striatum are the most susceptible to death. Neurons that project from the cortex to the striatum are also particularly affected, and reduction of the striatum and thinning of the cortex start a decade before the appearance of the symptoms. While alterations of the central nervous system (CNS) are the most prominent clinical features of HD, patients also suffer from metabolic and immune disturbances, skeletal-muscle wasting, weight loss, cardiac failure, testicular atrophy, and osteoporosis.

HD is caused by expansion of polyglutamine stretch with in the N-terminal domain of the huntingtin protein, caused by an abnormal expansion of a CAG repeat in exon 1 of the huntingtin gene. In the non-HD population, CAG is repeated 9 to times, with an average median of between 17 and 20 repeats. A CAG expansion exceeding 35 repeats results in HD. Rare carriers of 36 to 39 CAG repeats have lower penetrance and later onset of the disease than those with 40 or more CAG repeats. Homozygous patients show the same age at onset as heterozygotes, but disease progression can be more severe.

The disease-causing, mutant huntingtin protein (mhtt) form intracellular aggregates (both the nucleus and cytoplasm), which is found in the CNS and peripheral tissues (Cisbani and Cicchetti, Cell Death Dis 2012 Aug. 30; 3(8):e382.), causing neural death and other impacts on the periphery. For example, mhtt in the digestive tract may result in weight loss; mhtt in the muscle may result in muscular atrophy; mhtt in the endocrine system may result in impairment of insulin secretion and abnormal leptin release by adipose tissue; mhtt in the testes may cause decrease in testicular atrophy in germ cells; mhtt in the blood may cause abnormal immune responses such as inflammation, including increased production of cytokines (e.g., IL-4, IL-5, IL-6, IL-8, IL-10, and TNF-alpha) and/or increased susceptibility to apoptosis and/or autophagy and mitochondrial abnormalities in blood cells such as lymphocytes, macrophages, and/or monocytes; mhtt in the heart can cause heart failure, altered autonomic innervation, arrhythmia, and/or a coronary heart disease.

The “huntingtin” gene, also known as the “interesting transcript 15”, “IT5”, or “LOMARS” gene, is a gene that encodes the huntingtin protein. In humans, the huntingtin gene (described with the gene symbol “HTT”) is located on chromosome 4, with gene location 4p16.3 at nucleotide positions 3074681 to 3243960 (according to Gene Assembly GRCh38.p13), which encodes 67 exons (NCBI, Gene ID: 3064). In one aspect, HTT may have the polynucleotide sequence provided as NCBI Reference Sequence: NC_000004.12. For example, exon 5 corresponds to nucleotide positions 3105357 to 3105436 comprising the nucleic acid sequence of SEQ ID NO: 200; exon 8 corresponds to nucleotide positions 3116085 to 3116263 comprising the nucleic acid sequence of SEQ ID NO: 300; and exon 31 corresponds to nucleotide positions 3172908 to 3173131 comprising the nucleic acid sequence of SEQ ID NO: 400. In mice, the huntingtin gene (described with the gene symbol “Htt”) gene is located on chromosome 5, with gene location 5 B2; 5 17.92 cM at nucleotide positions 34919084 to 35069878 (according to Gene Assembly GRCm39), which encodes 67 exons (NCBI, Gene ID: 15194). In one aspect, Htt may have the polynucleotide sequence provided as NCBI Reference Sequence: NC_000071.7. For example, exon 31 corresponds to nucleotide positions 35004798 to 35005021 comprising the nucleic acid sequence of SEQ ID NO: 500.

The term “ionizable cationic lipid” as used herein, refers to any lipid that carries a net neutral charge at about physiological pH but is capable of becoming positively charged at a lower pH, e.g., pH below about 7, below about 6.5, below about 6, below about 5.5, below about 5, below about 4.5, below about 4, below about 3.5, or below about 3, typically at pH below about 6.5 or below about 6.5-7. Without wishing to be bound by theory, a net neutral charge helps toxicity, and positive charges under a low pH may be useful in forming a complex with a negatively charged cargo such as a nucleic acid molecule and/or protein. Becoming positive charges under as the pH decreases may also help release of the cargo from an endosome once in a cell (endosomal escape), e.g., by taking protons in an endosome thereby destabilizing and bursting the endosome. For example, N-dimethyl-2,3-dioleyloxy)propylamine (DODMA), 1,2-dioleoyl-3-dimethylammonium propane (“DODAP”), 1,2-Dilinoleoyl-3-dimethylaminopropane (DLinDAP), 1,2-DiLinoleyloxy-N,N-dimethylaminopropane (DLinDMA), N,N-dimethyl-2,2-di-(9Z,12Z)-9,12-octadecadien-1-yl-1,3-dioxolane-4-ethanamine (KC2), and (6Z,9Z,28Z,31Z)-heptatriaconta-6,9,28,31-tetraen-19-yl 4-(dimethylamino)butanoate (MC3) are ionizable cationic lipids.

The term “permanently cationic lipids” as used herein, refers to any lipid that carries a net positive charge without pKa or pKa>8. For example, N-(1-(2,3-dioleyloxyl)propyl)-N,N,N-trimethylammonium chloride (DOTMA), N-(1-(2,3-dioleyloxyl)propyl)-N-2-(sperminecarboxamido)ethyl)-N,N-dimethyl-ammonium trifluoroacetate (DOSPA) (which is a lipid component of Lipofectamine®), and N-(1-(2,3-dioleoyloxy)propyl)-N,N,N-trimethylammonium chloride (DOTAP) are permanently cationic lipids.

Examples of cationic lipids (including ionizable cationic lipids) may include, for example, N,N-dimethyl-2,3-dioleyloxy)propylamine (DODMA), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxyl)propyl)-N,N,N-trimethylammonium chloride (DOTMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAR.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLin-K-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3 aH-cyclopenta[d][1,3]dioxol-5-amine (ALNY-100), N,N-dimethyl-2,2-di-(9Z,12Z)-9,12-octadecadien-1-yl-1,3-dioxolane-4-ethanamine (KC2), (6Z,9Z,28Z,31Z)-heptatriaconta-6,9,28,31-tetraen-19-yl 4-(dimethylamino)butanoate (MC3), or a mixture thereof.

Additional examples of cationic lipids (including ionizable cationic lipids) include, but are not limited to, N-(2,3-dioleyloxyl)propyl-N,N-N-triethylammonium chloride (“DOTMA”); 1,2-Dioleyloxy-3-trimethylaminopropane chloride salt (“DOTAP.Cl”); 3.beta.-(N—(N′,N′-dimethylaminoethane)-carbamoyl)cholesterol (“DC-Chol”), N-(1-(2,3-dioleyloxyl)propyl)-N-2-(sperminecarboxamido)ethyl)-N,N-dimethyl-ammonium trifluoroacetate (“DOSPA”), dioctadecylamidoglycyl carboxyspermine (“DOGS”), 1,2-dioleoyl-3-dimethylammonium propane (“DODAP”), and N-(1,2-dimyristyloxyprop-3-yl)-N,N-dimethyl-N-hydroxyethyl ammonium bromide (“DMRIE”), and mixtures thereof. Additionally, a number of commercial preparations of cationic lipids can be used, such as, e.g., LIPOFECTIN® (available from GIBCO/BRL), and LIPOFECTAMINE® (available from GIBCO/BRL).

The term “complementary” or “complementarity” means that a nucleic acid can form hydrogen bond(s) with another nucleic acid sequence by either traditional Watson-Crick or other non-traditional types of interactions such as Wobble-base pairing which permits binding of guanine and uracil. A percent complementarity indicates the percentage of residues in a nucleic acid molecule that can form hydrogen bonds with a second nucleic acid sequence.

The term “mutation” or “point mutation” as used herein in relation to nucleic acid or nucleotide sequence means a change in a nucleotide in a DNA or RNA molecule. A mutation may be a change from a nucleotide to another nucleotide or deletion of a nucleotide or an insertion of a nucleotide. When a mutation causes replacement of a nucleotide with another nucleotide in an open reading frame, the mutation may cause an amino acid substitution (“missense mutation”) or appearance of an early stop codon (“nonsense mutation”) leading to a shorter protein product or may not cause any changes in the protein product (“silent mutation”). When a mutation causes insertion or deletion of a nucleotide in an open reading frame, unless the number of insertion or deletion is divisible by three, the mutation changes the grouping of the codons to be read (“frame shift mutation”), causing dramatic changes in the protein sequence.

“Lipid-based TCVs” as used in are TCVs that comprise at least one lipid and encompass lipid nanoparticles. In some embodiments, a lipid-based TCV may comprise at least one ionizable cationic lipid. In some embodiments, a lipid-based TCV may comprise at least one helper lipid. In some embodiments, a lipid-based TCV may comprise at least one phospholipid. In some embodiments, a lipid-based TCV may comprise at least one cholesterol (or cholesterol derivative). In some embodiments, a lipid-based TCV may comprise, essentially consist of, or consist of at least one ionizable cationic lipid, at least one helper lipid, at least one phospholipid, and at least one cholesterol (or cholesterol derivative), and optionally polyethylene glycol (PEG) or PEG-lipid. Exemplary TCVs include but not are limited to those described in Applicant's WO2020077007A1. In some embodiments, a lipid-based TCV may comprise, essentially consist of, or consist of an ionizable cationic lipid, one or more phospholipids, and cholesterol, the ratio of which are about 20:30:10:40 in mol %. In some embodiments, a lipid-based TCV may comprise, essentially consist of, or consist of an ionizable cationic lipid, one or more phospholipids, cholesterol, and PEG-lipid, the ratio of which are about 20:30:10:39:1 in mol %. TCVs may be generated using gentle mixing such as repeated manual reciprocation of the TCV-generating fluid in a pipette, staggered herringbone micromixer (SHM), T-junction mixing or extrusion methods, or other TCV-mixing methods as desired.

In some embodiments, a lipid-based TCV and/or a composition according to the present disclosure may substantially, essentially, or entirely lack organic solvents and/or detergents, which may help improve the stability and/or integrity of the TCV and/or its cargo. In some embodiments, the manufacturing method of a TCV according to the present disclosure may contribute to such a characteristic.

In some embodiments, a TCV and/or a composition may be stored at a freezing temperature. In some embodiments, when a TCV and/or a composition may be prepared, a cryoprotectant may be added. In some embodiments, a cryoprotectant may comprise a sugar-based molecule. Non-limiting examples of cryoprotectants include sucrose, trehalose, and a combination thereof. In certain embodiments, at least one cryoprotectant may be or may comprise a sugar-based molecule, e.g., a sugar molecule or a derivative thereof. In particular embodiments, the at least one cryoprotectant may be sucrose, trehalose, or a combination thereof. In a particular embodiment, the at least one cryoprotectant may be sucrose. In some embodiments, the concentration of the at least one cryoprotectant contained in the TCV or composition may be about 1% to about 40%, about 3% to about 30%, about 5% to about 30%, about 10% to about 20%, or about 15%.

In some embodiments, a TCV and/or a composition according to the present disclosure, which may comprise at least one cryoprotectant, may be stable at a freezing temperature, optionally at about −20° C. or about −80° C., optionally for at least about one week, at least about two weeks, at least about three weeks, at least about a month, at least about two months, at least about four months, at least about five months, at least about 6 months, at least about 9 months, at least about a year, or at least about two year, or longer, or about one week to about two year, about two weeks to about a year, about three weeks to about nine month, about one to about six months, about one to five months, about one to four months, about one to three months, or about one to two months.

A TCV according to the present disclosure may be prepared by any appropriate methods. In some embodiments, a TCV may be prepared by (a) generating a first solution by dissolving all components of the TCV in ethanol; (b) providing a second solution, which is aqueous; (c) combining the first and second solutions; and (d) removing ethanol, optionally by dialysis or evaporation. In some embodiments, the first solution in step (a) may contain the TCV lipid components at about 20-35 mM. In some embodiments, the second solution in step (b) may be an acidic buffer and optionally may contain acetate and/or citrate (e.g., sodium acetate and/or sodium citrate), which optionally may be at about 25 mM. In some embodiments, the pH of the second solution in step (b) may be about 3-8, about 4-7, about 3.5-4.5, or about 4. In some embodiments, the combining in step (c) may be by gentle mixing (optionally repeated manual reciprocation of the TCV-generating fluid in a pipette), mixing using a staggered herringbone micromixer (SHM), T-junction mixing, or extrusion. In a particular embodiment, the removing in step (d) is by dialysis. In a particular embodiment, the suspension resulting from step (c) may be dialyzed against an acidic buffer. In yet a particular embodiment, the acidic buffer may have a pH of about 3-5, about 3.5-4.5, or about 4. In some instances, the acidic buffer may contain acetate and/or citrate, such as sodium acetate and/sodium citrate. In a particular embodiment, the dialysis is performed against a 1000-fold volume of 25 mM sodium acetate (approximately pH 4) buffer.

Encapsulation of a cargo by a TCV may be performed by any appropriate methods. In some embodiments, wherein the TCV comprise a RNP as a cargo, the RNP encapsulation by TCVs may be performed by any appropriate methods. In some embodiments, the encapsulation may be performed by (i) providing an aqueous solution comprising the TCV, optionally in an acidic buffer (e.g., pH of about 3-5, about 3.5-4.5, or about 4); and (ii) mixing a RNP solution containing one or more RNPs with the aqueous solution. Mixing may be effected under conditions suitable for the at least one RNP to be encapsulate within the TCV. In some embodiments, the aqueous solution in step.

The term “nuclease” as used herein refers to an enzyme capable of catalyzing the cleavage of phosphodiester bonds between nucleotides of nucleic acids. An “endonuclease” cleaves phosphodiester bonds to separating nucleotides in a polynucleotide other than the two end nucleotides. In the CRISPR/Cas system, which involves a gRNA and a CRISPR-associated (Cas) endonuclease, the Cas endonuclease recognizes a PAM sequence in the target gene (sense or antisense) and if the gRNA is able to hybridize with a target sequence of the target gene proximate to the PAM sequence, the Cas endonuclease may mediate cleavage of the target gene at about 2-6 nucleotides upstream of the PAM. The PAM sequence is specific to the Cas endonuclease. Any appropriate Cas endonucleases may be used in the invention disclosed herein. Appropriate Cas endonucleases include but are not limited to Cas9 of different bacterial species such as Streptococcus pyogenes (SpCas9, which recognizes the PAM sequence of 5′-NGG-3′), Staphylococcus aureus Cas9 (SaCas9, which recognizes the PAM sequence of 5′-NNGRRT-3′), Streptococcus thermophilus (StCas9, which recognizes the PAM sequence of 5′-NGGNG-3′), Neisseria meningitidis (NmCas9, which recognizes the PAM sequence of 5′-NNNNGATT-3′), Francisella novicida (FnCas9, which recognizes the PAM sequence of 5′-NG-3′), Campylobacter jejuni (CjCas9), which recognizes the PAM sequence of 5′-NNNNACA-3′), Streptococcus canis (ScCas9, which recognizes the PAM sequence of 5′-NNGG-3′), Staphylococcus auricularis (SauriCas9, which recognizes the PAM sequence of 5′-NNG-3′), or any engineered variants thereof, including but not limited to SaCas9-HF, SpCas9-HF1, KKHSaCas9, eSpCas9, HypaCas9, FokI-Fused dCas9, xCas9, SpRY (variant of SpCas9), SpG (variant of SpCas9), which are collectively referred to as Cas9 herein. Other Cas endonuclease examples include Cas3, Cas8a2, Cas8b, Cas8c, Cas10, Cas11, Cas12, Cas12a or Cpf1, Cas13, Cas13a, C2c1, C2c3, and C2c2. In some embodiments, Cas9 may be a wild-type SpCas, e.g., comprising SEQ ID NO: 600 or its variant, e.g., comprising any of SEQ ID NOS: 601-611.

The terms “nucleic acid”, “nucleic acid molecule”, and “polynucleotide” are used interchangeably herein and encompass any compounds that comprise a polymer of nucleotides linked via a phosphodiester bond. Exemplary nucleic acids include but are not limited to RNA and DNA molecules, including molecules comprising cDNA, genomic DNA, synthetic DNA, and DNA or RNA molecules containing nucleic acid analogs. Nucleic acid molecules can have any three-dimensional structure. A nucleic acid molecule can be double-stranded or single-stranded (e.g., a sense strand or an antisense strand). Other non-limiting examples of nucleic acid molecules include genes, gene fragments, exons, introns, messenger RNA (mRNA), transfer RNA, ribosomal RNA, siRNA, micro-RNA, tracrRNAs, crRNAs, guide RNAs, ribozymes, cDNA, recombinant polynucleotides, branched polynucleotides, nucleic acid probes and nucleic acid primers. A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

The term “phospholipid” as used herein refers to any lipid comprising a phosphate group. Non-limiting examples of suitable phospholipids include: distearoylphosphatidylcholine (DSPC), dioleoyl phosphatidylethanolamine (DOPE), dipalmitoylphosphatidylcholine (DPPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and combinations thereof. In one embodiment, the phospholipid is distearoylphosphatidylcholine (DSPC).

The term “polyethylene glycol-lipid” or “PEG-lipid” as used herein refers to any lipid modified or conjugated to one or more polyethylene glycol (PEG) molecules. Without wishing to be bound by theory, containing PEG or a PEG-lipid in a TCV may help maintain TCV particle size (keep a TCV from getting too big) and/or help maintain particle stability in vivo. Some examples of PEG-lipids that are useful in the present invention may have a variety of “anchoring” lipid portions to secure the PEG to the surface of the lipid-based TCVs. Non-limiting examples of suitable PEG-lipids include PEG-myristoyl diglyceride (PEG-DMG) (e.g., 1,2-dimyristoyl-rac-glycero-3-methoxypolyethylene glycol-2000 (Avanti® Polar Lipids (Birmingham, AL)), which is a mixture of 1,2-DMG PEG2000 and 1,3-DMG PEG2000 (e.g., in about 97:3 ratio)), PEG-phosphatidylethanolamine and phosphatidic acid, PEG-ceramide conjugates (e.g., PEG-CerC14 or PEG-CerC20) which are described in U.S. Pat. No. 5,820,873, incorporated herein by reference, PEG-modified dialkylamines, and PEG-modified 1,2-diacyloxypropan-3-amines. Particularly examples include PEG-modified diacylglycerols and dialkylglycerols.

The phrase “pharmaceutically acceptable” refers to molecular entities and compositions that are physiologically tolerable and do not typically produce an unintended and intolerable response such as an allergic response, when administered to a human. In certain embodiments, the term “pharmaceutically acceptable”, as used herein, means approved by a regulatory agency of the Federal or a state government or listed in the U.S. Pharmacopeia or other generally recognized pharmacopeia for use in animals, and more particularly in humans. The phrase “pharmaceutically acceptable carrier” refers to substances or collections of substances capable of being combined with an active ingredient that is suitable for use in contact with the cells or tissues of mammals for purposes of a therapeutic treatment in the mammals under anticipated exposure conditions.

The term “ribonucleoprotein”, “RNP”, or “RNP complex” as used herein refers to a complex of one or more RNA molecules and an RNA-binding protein. In the context of the CRISPR/Cas system, an RNP may be a complex of a gRNA and a Cas endonuclease. The gRNA may be for example a sgRNA or a dgRNA. Such a RNP may be generated by any appropriate methods. In some embodiments, the RNP may be formed by mixing Cas9 and gRNA at an approximately equimolar ratio, optionally for about 5 minutes.

“Single-strand oligo DNA nucleotides” or “ssODN” as used herein refers to a short DNA fragment of a single strand comprising a particular polynucleotide sequence that may be useful for some of the embodiments disclosed herein. In one aspect, ssODN may be used as part of CRISPR/Cas-mediated gene editing disclosed herein and may function as a DNA template (may also referred to as a DNA repair template) to mediate a knock-in of a sequence of interest through the Cas9-mediated double-strand break site. Such a knock-in may be via homology-directed repair (HDR). In some embodiments, a ssODN may have homology to the strand that initiates repair in the direction of a desired modification. In some embodiments, a ssODN may comprise (i) a 5′ homology arm and (ii) a 3′ homology arm, and optionally (iii) a central region comprising one or more desired nucleic acids, sandwiched by the 5′ homology arm and the 3′ homology arm. Such a homology arm may comprise approximately 10-2500 nucleotides (nt). 5′ and 3′ homology arms often have the same or similar nucleotide lengths (e.g., 0 or 1 to 10 nt difference), but 5′ and 3′ homology arms that significantly differ in length may also be used as long as the ssODN mediate and/or assist an intended gene repair. 5′ and/or 3′ homology arms may be 100% complementary to the corresponding sequence in the original DNA sequence before gene editing or may have one or more (a few) mutations (e.g., silent mutation) relative to the corresponding sequence in the original DNA sequence before gene editing. In some embodiments, ssODN may have one or more mutations at the PAM sequence (or its reverse (or antisense) sequence of to the PAM sequence, i.e., the opposite strand) and/or at one or more of the 5′-neighbouring bases of the PAM (or the 3′-neighbouring bases of the reverse (or antisense) sequence corresponding to the PAM). In some cases, such a mutation(s) helps prevent or reduce Cas-mediated cleavage of the ssODN itself or of a gene-edited DNA molecule. In some embodiments, a ssODN may comprise complementarity to the gRNA strand. In some embodiments, a ssODN may comprise a total length of approximately 40-5000 nucleotides (nt). As a template DNA, a double-stranded DNA template may also be used instead. In such a case, one of the strands of the template DNA may comprise the same sequence as a desired ssODN and the other strand has a sequence complementary thereto.

A “subject” as used herein, which may be interchangeably referred to as “patient”, “individual”, or “animal”, refers to a vertebrate including members of the mammalian species, such as canine, feline, lupine, mustela, rodent (racine, murine, etc.), equine, bovine, ovine, caprine, porcine species, and primates including humans. In specific embodiments, the subject is a human. In some embodiments, a subject may have or have a risk of developing a target disease. In specific embodiments, a subject may have or have a risk of developing HD.

The term “target cell” or “host cell” as used herein refers to a cell in which the cargo of a TCV according to the present disclosure is intended to function. A TCV according to the present disclosure may be engineered to specifically carry its cargo in a target cell, for example by comprising one or more targeting moiety on the surface.

The term “target disease”, as used herein, which may be used interchangeably with “target disorder” or “target condition”, refers to a disease, disease, or condition that a TCV containing a cargo or a composition containing such a TCV according to the present disclosure is intended to treat, prevent, or ameliorate. A TCV according to the present disclosure may carry its cargo into a target cell, thereby altering a target gene or target gene expression and thus prevent, treat, or ameliorate a target disease.

The term “target gene” or “target gene of interest” as used herein is a gene (including the gene itself and in some cases a polynucleotide region that regulates the expression of the gene such as a promoter and/or an enhancer of the gene) whose sequence is to be altered (e.g., disrupted, partially or entirely removed, or partially or entirely replaced with an intended sequence, for example by a endonuclease (such as Cas9) and a guide RNA) by a cargo of a TCV according to the present disclosure. In general, “target gene” may be any gene of interest in a target cell. The sequence of “target gene” may be the sense strand sequence or the antisense strand sequence of the gene.

The term “target sequence” or “target polynucleotide sequence” as used herein is the sequence of a polynucleotide that a targeting sequence of a gRNA according to the present disclosure may interact with in a cell. A target sequence may be fully complementary with a targeting sequence or there may be one or more mismatches. In some embodiments, there may be one, two, three, four, or five mismatches. In some embodiments, a target sequence and a targeting sequence may share 100% sequence identity, about 99% sequence identity, about 98% sequence identity, about 97% sequence identity, about 96% sequence identity, about 95% sequence identity, about 94% sequence identity, about 93% sequence identity, about 92% sequence identity, about 91% sequence identity, about 90% identity, about 89% sequence identity, about 87% sequence identity, about 86% sequence identity, about 85% sequence identity, about 84% sequence identity, about 83% sequence identity, about 82% sequence identity, about 81% sequence identity, about 80% identity, about 79% sequence identity, about 78% sequence identity, about 77% sequence identity, about 76% sequence identity, or about 75% sequence identity.

The term “therapeutically effective amount/dose” refers to the quantity of a TCV or a pharmaceutical composition comprising such a TCV or its cargo that is sufficient to provide a therapeutic effect (which may be based on, e.g., the number or percentage of target cells in which the intended target gene alteration occurred, the overall change in the target gene expression, the amelioration of one or more symptom, the number or percentage of target cells exhibiting an intended phenotype such as morphology, etc) upon administration to a subject.

The term “transfection competent vesicle” or “TCV” as used herein, in its broadest sense, encompasses any materials capable of carrying one or more cargoes, such as but not limited to a nucleic acid molecule (e.g., a DNA or a RNA) and/or a nucleic acid molecule complexed with a protein or peptide, into a cell. Examples of TCVs include but are not limited to: compounds, such as calcium phosphate, polycations, cationic lipids, phospholipids, organic and nonorganic polymers, dendrimers, organic and nonorganic nanoparticles and nanobeads, and any combinations thereof, lipid-based compositions capable of carrying a nucleic acid molecule, such as liposomes and lipid nanoparticles (LNPs); plasmids; virus-like particles (VLPs); and viral vectors, such as retroviral, lentiviral, and adenoviral vectors. In some embodiments, a TCV may comprise a targeting moiety (e.g., antibody or antibody fragment such as a Fab fragment), which allows the TCV to carry its cargo preferentially into a target cell.

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. In specific embodiments, the effect of the “treatment” may be evaluated by the amelioration of at least one measurable physical parameter of a disease, resulting from the administration of one or more therapies. The parameter may be, for example, gene expression profiles, the number of disease-affected cells, the percentage or frequency of disease-affected cells among the cells of the same lineage, disease-associated marker levels, and/or the presence or absence or levels of certain cytokines or chemokines or other disease-associated molecules and may not necessarily discernible by the patient. In some embodiments “treat”, “treatment,” or “treating” may result in and/or be evaluated based on the inhibition of the progression of a disease, either physically by, e.g., stabilization of a discernible symptom, physiologically by, e.g., stabilization of a physical parameter, or both. In some embodiments the terms “treat”, “treatment” and “treating” refer to the reduction or stabilization of cancerous tissue or cells. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

A “vector” is a compound or a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, viruses, and virus-like particles (VLPs). Thus, the term “vector” includes an autonomously replicating plasmid, a self-replicating RNA, or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-articles refers to groups having 1, 2, 3, 4, or 5 articles, and so forth.

The scope of the present devices, systems and methods, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “step” is specifically recited in a claim.

It should be understood that, unless clearly indicated otherwise, in any methods disclosed or claimed herein that comprise more than one step, the order of the steps to be performed is not restricted by the order of the steps specifically cited.

From the foregoing, it will be appreciated that, although specific embodiments of the present invention referring to certain molecules, compositions, methods, or protocols, etc., have been discussed herein for purposes of illustration, various modifications may be made without deviating from the spirit and scope of the discussion herein. Accordingly, it is to be understood that the present invention is not limited to such specific embodiments and rather also encompass modifications thereof as well as all permutations and combinations of the subject matter set forth herein. It is also to be understood that the terminology used in the description is for the purpose of describing the particular versions or embodiments only and is not intended to limit the scope of the present invention which will be limited only by the appended claims or other claims supported by the disclosure herein.

### EXEMPLARY EMBODIMENTS

Described herein below are some more exemplary embodiments according to the present disclosure.
Embodiment 1. A composition comprising a mixture of multiple different guide RNAs (gRNAs) complexed to RNA-guided endonucleases to provide a plurality of different ribonucleoprotein complexes (RNP complexes), wherein the RNP complexes are contained within lipid-based transfection competent vesicles (TCVs), wherein the TCVs comprise ionizable cationic lipids and the composition lacks any solvent or detergent.
Embodiment 2. The composition of Embodiment 1 wherein the multiple different guide RNAs are all directed to a same region of a target disease-associated nucleic acid sequence.
Embodiment 3. The composition of Embodiment 2 wherein the target disease-associated nucleic acid sequence is a gene.
Embodiment 4. The composition of Embodiment 3 wherein the gene is a native gene.
Embodiment 5. The composition of Embodiment 4 wherein the native gene is a mutant gene associated with causing a disease or condition in the animal.
Embodiment 6. The composition of Embodiment 5 wherein the disease or condition is Huntington's Disease.
Embodiment 7. The composition of any one of Embodiments 2 to 6 wherein the composition comprises at least three different guide RNAs (gRNAs) directed to the same target nucleic acid sequence.
Embodiment 8. The composition of any one of Embodiments 1 to 7 wherein the guide RNAs comprise at least two different complementary regions that are each complementary to at least a portion of exon (31) of the HTT gene.
Embodiment 9. The composition of any one of Embodiments 1 to 7 wherein the guide RNAs comprise at least three different complementary regions, wherein each complementary region is at least 85% complementary to SEQ ID no. 1, SEQ ID no. 2 and SEQ ID no. 3, respectively.
Embodiment 10. The composition of any one of Embodiments 1 to 7 wherein the guide RNAs comprise at least three different complementary regions, wherein each complementary region is at least 85% complementary to SEQ ID no. 4, SEQ ID no. 5 and SEQ ID no. 6, respectively.
Embodiment 11. The composition of any one of Embodiments 1 to 7 wherein the guide RNAs comprise at least three different complementary regions, wherein each complementary region is at least 85% complementary to SEQ ID no. 7, SEQ ID no. 8 and SEQ ID no. 9, respectively.
Embodiment 12. The composition of any one of Embodiments 1 to 7 wherein the guide RNAs comprise at least two different complementary regions selected from SEQ ID no. 1 to SEQ ID no. 9, wherein each complementary region in the guide RNA is at least 85% complementary to the respective different complementary regions selected from SEQ ID no. 1 to SEQ ID no. 9.
Embodiment 13. The composition of any one of Embodiments 1 to 7 wherein the guide RNAs comprise at 3 to 15 different complementary regions that are complementary to different regions of the HTT gene.
Embodiment 14. The composition of any one of Embodiments 1 to 13 wherein the nuclease comprises at least one of Cas9 Cpf1, Cas12a, CasX or dCas9.
Embodiment 15. The composition of any one of Embodiments 1 to 14 wherein the TCV comprises a mixture of 1,2-Dioleyloxy-3-dimethylamino-propane (DODMA), 1,2-dioleoyl-sn-glycero-3-phosphoethanolamine (DOPE), 1,2-Distearoyl-sn-glycero-3-phosphocholine (DSPC), and cholesterol (Chol) at about 20/30/10/40 mol %, respectively.
Embodiment 16. The composition of any one of Embodiments 1 to 15 wherein the guide RNA sequence comprises at least two of SEQ ID nos. 1-9.
Embodiment 17. A method of altering a gene expression level in an animal comprising administering to the animal a therapeutically effective amount of a therapeutic agent configured to selectively alter expression of a target disease-associated nucleic acid sequence in the animal, wherein the therapeutic agent comprises a cationic lipid-based transfection competent vesicle (TCV) containing a ribonucleoprotein (RNP), wherein:
the TCV comprises ionizable cationic lipids without any solvent, and the RNP comprises an RNA-guided endonuclease, and
wherein the endonuclease was mixed with at least a first guide RNA (gRNA) comprising a first guide nucleotide sequence configured to guide the nuclease to the target disease-associated nucleic acid sequence and a second guide RNA (gRNA) comprising a second nucleotide sequence different from the first nucleotide sequence, the second nucleotide sequence configured to guide the nuclease to the target disease-associated nucleic acid sequence to provide RNP complexes, and then the RNP complexes are then mixed with TCVs, wherein
the mixing of the TCVs with the RNP complexes are performed without the presence of an organic solvent or detergent under conditions suitable and for a time sufficient for the RNP complexes to encapsulate within the TCV to provide the ionizable cationic lipid-based TCV containing the RNP complexes and first guide and second guide without any organic solvent or detergent within the TCV.
Embodiment 18. The method of Embodiment 17 wherein the target disease-associated nucleic acid sequence is a gene.
Embodiment 19. The method of Embodiment 18 wherein the gene is a native gene.
Embodiment 20. The method of Embodiment 18 wherein the native gene is a mutant gene associated with causing a disease or condition in the animal.
Embodiment 21. The method of Embodiment 18 wherein the disease or condition is Huntington's Disease.
Embodiment 22. The method of Embodiment 18 wherein the TCV further comprises a third guide RNA (gRNA) comprising a third nucleotide sequence different from the first nucleotide sequence and the second nucleotide sequence.
Embodiment 23. The method of any one of Embodiments 17 to 22 wherein the first and second guide RNAs comprise at least two different complementary regions that are each complementary to at least a portion of exon (31) of the HTT gene.
Embodiment 24. The method of any one of Embodiments 17 to 23 wherein the guide RNA comprises at least three different complementary regions, wherein each complementary region is at least 85% complementary to SEQ ID no. 1, SEQ ID no. 2 and SEQ ID no. 3, respectively.
Embodiment 25. The method of any one of Embodiments 17 to 23 wherein the guide RNA comprises at least three different complementary regions, wherein each complementary region is at least 85% complementary to SEQ ID no. 4, SEQ ID no. 5 and SEQ ID no. 6, respectively.
Embodiment 26. The method of any one of Embodiments 17 to 23 wherein the guide RNA comprises at least three different complementary regions, wherein each complementary region is at least 85% complementary to SEQ ID no. 7, SEQ ID no. 8 and SEQ ID no. 9, respectively.
Embodiment 27. The method of any one of Embodiments 17 to 23 wherein the guide RNA comprises at least two different complementary regions selected from SEQ ID no. 1 to SEQ ID no. 9, wherein each complementary region in the guide RNA is at least 85% complementary to the respective different complementary regions selected from SEQ ID no. 1 to SEQ ID no. 9.
Embodiment 28. The method of any one of Embodiments 17 to 23 wherein the guide RNA comprises 3 to 15 different complementary regions that are complementary to different regions of the HTT gene.
Embodiment 29. The method of any one of Embodiments 17 to 28 wherein the nuclease comprises Cas9 Cpf1, Cas12a, CasX, dCas9.
Embodiment 30. The method of any one of Embodiments 17 to 29 wherein the TCV comprises a mixture of 1,2-Dioleyloxy-3-dimethylamino-propane (DODMA), 1,2-dioleoyl-sn-glycero-3-phosphoethanolamine (DOPE), 1,2-Distearoyl-sn-glycero-3-phosphocholine (DSPC), and cholesterol (Chol) at about 20/30/10/40 mol %, respectively.
Embodiment 31. The method of Embodiment 17 wherein the guide RNA sequence comprises at least one of SEQ ID nos. 1-9.
Embodiment 32. The method of any one of Embodiments 17 to 31 wherein the animal is a mammal.
Embodiment 33. The method of any one of Embodiments 17 to 32 wherein the wherein the method further comprises at least two different administrations to the animal of the therapeutic agent comprising the cationic lipid-based TCV containing the RNP.
Embodiment 34. The method of Embodiment 33 wherein the at least two administrations occur sequentially at different points in time.
Embodiment 35. The method of Embodiment 33 or 34 wherein the at least two administrations occur simultaneously at different locations on the body of the animal.
Embodiment 36. The method of any one of Embodiments 17 to 35 wherein the administrating is into a central nervous system of the animal.
Embodiment 37. The method of Embodiment 36 wherein the administrating is via direct injection into a central nervous system of the animal.
Embodiment 38. The method of any one of Embodiments 17 to 37 wherein the administrating targets a cell located within a brain of the animal.
Embodiment 39. The method of any one of Embodiments 17 to 38 wherein the administrating results in amelioration or halt in progression of one of more symptoms of Huntington's Disease in a patient.
Embodiment 40. The method of any one of Embodiments 17 to 39 wherein the water-based solution is a 25 mM to 100 mM acetate buffer.
Embodiment 41. The method of any one of Embodiments 17 to 40 wherein the lipid-based TCV is empty prior to the encapsulation.
Embodiment 42. The method of Embodiment 41 wherein the cationic lipid comprises an ionizable cationic lipid.
Embodiment 43. The method of any one of Embodiments 17 to 42 wherein the lipid-based TCV comprises about 20 mol % to 50 mol % cationic lipid.
Embodiment 44. The method of Embodiment 42 wherein the ionizable cationic lipid comprises 1,2-Dioleyloxy-3-dimethylamino-propane (DODMA).
Embodiment 45. The method of Embodiment 42 wherein the lipid-based TCV comprises a mixture of 1,2-Dioleyloxy-3-dimethylamino-propane (DODMA), 1,2-dioleoyl-sn-glycero-3-phosphocholine (DOPC), 1,2-dioleoyl-sn-glycero-3-phosphoethanolamine (DOPE), and 1,2-distearoyl-sn-glycero-3-phosphocholine (DSPC).
Embodiment 46. The method of any one of Embodiments 17 to 45 wherein the mixture further comprises at least one of polyethylene glycol (PEG) or cholesterol.
Embodiment 47. The method of any one of Embodiments 17 to 46 wherein the selected cargo comprises a protein.
Embodiment 48. The method of Embodiment 47 wherein the RNP is a functional ribonucleoprotein.
Embodiment 49. The method of Embodiment 47 or Embodiment 48 wherein the RNP comprises at least one of a Cas9 protein or a guide RNA.
Embodiment 50. The method of Embodiment 47 or Embodiment 48 wherein the RNP comprises both a Cas9 protein and a guide RNA.
Embodiment 51. The method of any one of Embodiments 17 to 50 wherein the lipid-based TCV and the selected cargo are mixed at an about 473:1 to 1173:1 molar ratio of lipid-based TCV:selected cargo.
Embodiment 52. The method of any one of Embodiments 17 to 50 wherein the lipid-based TCV and the selected cargo are mixed at an about 473:1 to 5000:1 molar ratio of lipid-based TCV:selected cargo.
Embodiment 53. The method of any one of Embodiments 17 to 52 wherein the selected cargo is a ribonucleoprotein (RNP).
Embodiment 54. The method of any one of Embodiments 17 to 53 wherein the mixing is performed using staggered herringbone micromixing or T-junction mixing.
Embodiment 55. The method of any one of Embodiments 17 to 54 wherein the mixing is performed using extrusion mixing.
Embodiment 56. The method of any one of Embodiments 17 to 54 wherein the mixing is performed via reciprocation in a pipette.
Embodiment 57. A method of transfection, the method comprising transfecting a target cell with a lipid-based transfection competent vesicle (TCV)-encapsulated selected cargo produced according to any one of Embodiments 17 to 56.
Embodiment 58. The method of Embodiment 57 wherein the target cell is a mammalian cell.
Embodiment 59. The method of Embodiment 57 wherein the target cell is a mammalian primary neuronal cell.
Embodiment 60. The method of Embodiment 57 wherein the target cell is a cell from a mammalian patient.
Embodiment 61. The method of any one of Embodiments 57 to 60 wherein the method is performed in a laboratory.
Embodiment 62. The method of any one of Embodiments 57 to 60 wherein the method is performed in a factory to produce commercial quantities of transfected cells.
Embodiment 63. The method of any one of Embodiments 57 to 60 wherein the method is performed as a part of an in vivo procedure.
Embodiment 64. The method of any one of Embodiments 57 to 60 wherein the method is performed as a part of a medical procedure.
Embodiment 65. The method of any one of Embodiments 57 to 60 wherein the method is performed as a part of a therapeutic procedure.
Embodiment 66. The method of any one of Embodiments 57 to 60 wherein the method is performed as a part of a gene therapy procedure.
Embodiment 67. The method of any one of Embodiments 57 to 60 wherein the method is performed as a part of treating Huntington's disease.
Embodiment 68. The method of any one of Embodiments 57 to 67 wherein the method further comprises delivering the lipid-based TCV-encapsulated selected cargo to a brain of the patient.
Embodiment 69. A kit comprising the composition of any one of Embodiments 1 to 17, wherein the composition is in a vessel and the kit comprises instructions for use of the composition.
Embodiment 70. The kit of Embodiment 68 wherein the instructions direct use of the composition according to any one of Embodiments 57 to 63.
Embodiment 71. The kit of Embodiment 69 or 70 wherein the vessel is configured to administer at least one dose of the composition to a mammal, the kit further comprising at least one label comprising instructions for the administration.
Embodiment 72. An isolated and purified composition according to any one of Embodiments 1 to 17 for use in the manufacture of a medicament for inhibiting, preventing, or treating a disease or condition in a patient.
Embodiment 73. The composition of Embodiment 72 wherein the patient is a mammal.

What is claimed is:

1. An isolated guide RNA (gRNA) for Clustered Regularly Interspaced Short Palindromic Repeats (CRISPR)-mediated gene editing, wherein the gRNA comprises a targeting sequence comprising at least or consisting of 17 nucleotides, optionally 17, 18, 19, 20, 21, 22, 23, 24, 25, 26, 27, 28, 29, or 30 nucleotides, wherein:
(I) the targeting sequence comprises or consists of:
(i) (i-1) a sequence of at least 17 consecutive nucleotides, optionally 17, 18, 19, 20, 21, 22, 23, 24, 25, 26, 27, 28, 29, or 30 consequent nucleotides, contained in one or more of SEQ ID NO: 200, 201, 202, 205, 206, and/or 207, and/or (i-2) a sequence of:
(a) SEQ ID NO: 4, 210, 211, 212, or 213,
(b) SEQ ID NO: 5, 220, 221, 222, or 223, or
(c) SEQ ID NO: 6, 230, 231, 232, or 233,
(ii) a sequence of at least 17 nucleotides comprising one or more mutations, optionally one, two, three, four, or five mutations, said mutations relative to the at least 17 consecutive nucleotides, optionally 17, 18, 19, 20, 21, 22, 23, 24, 25, 26, 27, 28, 29, or 30 consequent nucleotides, of (i), optionally wherein the one or more mutations are at any nucleotide position(s) or are at position(s) other than the 4th to the 7th nucleotide positions from the 3′-end of the at least 17 consecutive nucleotides, or
(iii) a sequence of at least 17 nucleotides which comprises at least 85, 90, 95, 96, 97, 98 or 99% sequence identity to the sequence of at least 17 consecutive nucleotides, optionally 17, 18, 19, 20, 21, 22, 23, 24, 25, 26, 27, 28, 29, or 30 consecutive nucleotides, of (i),
optionally wherein the at least 17 consecutive nucleotides are;
(a) immediately upstream of a protospacer adjacent motif (PAM) or protospacer flanking site (PFS) of a CRISPR-associated (Cas) endonuclease in one or more of SEQ ID NO: 200, 201, 202, 205, 206, and/or 207, optionally wherein the Cas endonuclease is Cas9, further optionally wherein the Cas endonuclease is Streptococcus pyogenes Cas9 (SpCas9) and/or the PAM sequence is 5′-NGG-3′ wherein N represents any nucleotide; or
(b) immediately downstream of the PAM or PFS of a Cas endonuclease in one or more of SEQ ID NO: 200, 201, 202, 205, 206, and/or 207, optionally wherein the Cas endonuclease is Cpf1 and/or the PAM sequence is 5′-TTTN-3′ wherein N represents any nucleotide; and/or
(II) when the gRNA is complexed with a Cas endonuclease, the targeting sequence guides the Cas endonuclease to allow for cleavage of exon 5 of the huntingtin (HTT) gene or within 30 nucleotides upstream or downstream of the exon 5, optionally wherein:
(i) the cleavage is within one or more of SEQ ID NOS: 200, 201, 202, 205, 206, and/or 207, optionally at any one or more of the following positions:
(i-1) between the 9^{th} and 10^{th} nucleotides from the 5′ end of SEQ ID NO: 200,
(i-2) between the 55^{th} and 56^{th} nucleotides from the 5′ end of SEQ ID NO: 200,
(i-3) between the 72^{th} and 73^{th} nucleotides from the 5′ end of SEQ ID NO: 200,
(i-4) between the 9^{th} and 10^{th} nucleotides from the 3′ end of SEQ ID NO: 205,
(i-5) between the 55^{th} and 56^{th} nucleotides from the 3′ end of SEQ ID NO: 205, and/or
(i-6) between the 72^{th} and 73^{th} nucleotides from the 3′ end of SEQ ID NO: 200; and/or
(ii) the Cas endonuclease is Cas 9, Cas3, Cas8a2, Cas8b, Cas8c, Cas10, Cas11, Cas12, Cas12a or Cpf1, Cas13, Cas13a, C2c1, C2c3, or C2c2, further optionally Cas9 or Cpf1, yet further optionally SpCas9,
optionally wherein the gRNA comprises one or more of the following features:
(A) (A-1) the gRNA is a single guide RNA (sgRNA) comprising in a single strand:
(i) a CRISPR RNA (crRNA) sequence comprising the targeting sequence and a crRNA backbone sequence, and
(ii) a trans-activating CRISPR RNA (tracrRNA) sequence,
optionally wherein the crRNA sequence and the tracrRNA sequence are linked via a linker optionally comprising SEQ ID NO: 109, further optionally wherein the gRNA comprises the targeting sequence followed by, optionally immediately followed by, a sgRNA backbone sequence of any of SEQ ID NOS: 111-114, optionally wherein the sgRNA backbone sequence is followed by one or more uracils, further optionally 1-10 uracils, or
(A-2) the gRNA is a dual guide RNA (dgRNA) formed by hybridization between:
(i) a crRNA comprising the targeting sequence and a crRNA backbone sequence, and
(ii) a tracrRNA,
optionally wherein:
(a) the targeting sequence is followed by, optionally immediately followed by, a sgRNA backbone sequence of SEQ ID NOS: 115 and the tracrRNA comprises SEQ ID NO: 116, or
(b) the targeting sequence is followed by, optionally immediately followed by, a sgRNA backbone sequence of SEQ ID NOS: 117 and the tracrRNA comprises SEQ ID NO: 118;
(B) the gRNA is synthetic or recombinant; and/or
(C) the gRNA is a synthetic sgRNA and comprises at least one chemical modification, which is optionally:
(C-1) 2-O-methylation further optionally at first three and last three bases, and/or
(C-2) one or more 3′ phosphorothioate bonds, further optionally between first three and last two bases.

2. An isolated gRNA for CRISPR-mediated gene editing, wherein the gRNA comprises a targeting sequence comprising at least or consisting of 17 nucleotides, optionally 17, 18, 19, 20, 21, 22, 23, 24, 25, 26, 27, 28, 29, or 30 nucleotides, wherein:
(I) the targeting sequence comprises or consists of:
(i) (i-1) a sequence of at least 17 consecutive nucleotides, optionally 17, 18, 19, 20, 21, 22, 23, 24, 25, 26, 27, 28, 29, or 30 consequent nucleotides, contained in one or more of SEQ ID NO: 300, 301, 302, 305, 306, and/or 307, and/or (i-2) a sequence of:
(a) SEQ ID NO: 7, 310, 311, 312, or 313,
(b) SEQ ID NO: 8, 320, 321, 322, or 323, or
(c) SEQ ID NO: 9, 330, 331, 332, or 333,
(ii) a sequence of at least 17 nucleotides comprising one or more mutations, optionally one, two, three, four, or five mutations, said mutations relative to the at least 17 consecutive nucleotides, optionally 17, 18, 19, 20, 21, 22, 23, 24, 25, 26, 27, 28, 29, or 30 consequent nucleotides, of (i), optionally wherein the one or more mutations are at any nucleotide position(s) or are at position(s) other than the 4th to the 7th nucleotide positions from the 3′-end of the at least 17 consecutive nucleotides, or
(iii) a sequence of at least 17 nucleotides which comprises at least 85, 90, 95, 96, 97, 98 or 99% sequence identity to the sequence of at least 17 consecutive nucleotides, optionally 17, 18, 19, 20, 21, 22, 23, 24, 25, 26, 27, 28, 29, or 30 consecutive nucleotides, of (i),
optionally wherein the at least 17 consecutive nucleotides are;
(a) immediately upstream of a PAM or PFS of a Cas endonuclease in one or more of SEQ ID NO: 300, 301, 302, 305, 306, and/or 307, optionally wherein the Cas endonuclease is Cas9, further optionally wherein the Cas endonuclease is SpCas9 and/or the PAM sequence is 5′-NGG-3′ wherein N represents any nucleotide; or
(b) immediately downstream of the PAM or PFS of a Cas endonuclease in one or more of SEQ ID NO: 300, 301, 302, 305, 306, and/or 307, optionally wherein the Cas endonuclease is Cpf1 and/or the PAM sequence is 5′-TTTN-3′ wherein N represents any nucleotide; and/or
(II) when the gRNA is complexed with a Cas endonuclease, the targeting sequence guides the Cas endonuclease to allow for cleavage of exon 8 of the HTT gene or within 30 nucleotides upstream or downstream of the exon 8, optionally wherein:
(i) the cleavage is within one or more of SEQ ID NOS: 300, 301, 302, 305, 306, and/or 307, optionally at any one or more of the following positions:
(i-1) between the 15^{th} and 16^{th} nucleotides from the 5′ end of SEQ ID NO: 300,
(i-2) between the 69^{th} and 70^{th} nucleotides from the 5′ end of SEQ ID NO: 300,
(i-3) between the 112^{th} and 113^{th} nucleotides from the 5′ end of SEQ ID NO: 300,
(i-4) between the 15^{th} and 16^{th} nucleotides from the 3′ end of SEQ ID NO: 305,
(i-5) between the 69^{th} and 70^{th} nucleotides from the 3′ end of SEQ ID NO: 305, and/or
(i-6) between the 112^{th} and 113^{th} nucleotides from the 3′ end of SEQ ID NO: 305; and/or
(ii) the Cas endonuclease is Cas 9, Cas3, Cas8a2, Cas8b, Cas8c, Cas10, Cas11, Cas12, Cas12a or Cpf1, Cas13, Cas13a, C2c1, C2c3, or C2c2, further optionally Cas9 or Cpf1, yet further optionally SpCas9,
optionally wherein the gRNA comprises one or more of the following features:
(A) (A-1) the gRNA is a sgRNA comprising in a single strand:
(i) a crRNA sequence comprising the targeting sequence and a crRNA backbone sequence, and
(ii) a tracrRNA sequence,
optionally wherein the crRNA sequence and the tracrRNA sequence are linked via a linker optionally comprising SEQ ID NO: 109, further optionally wherein the gRNA comprises the targeting sequence followed by, optionally immediately followed by, a sgRNA backbone sequence of any of SEQ ID NOS: 111-114, optionally wherein the sgRNA backbone sequence is followed by one or more uracils, further optionally 1-10 uracils, or
(A-2) the gRNA is a dgRNA formed by hybridization between:
(i) a crRNA comprising the targeting sequence and a crRNA backbone sequence, and
(ii) a tracrRNA,
optionally wherein:
(a) the targeting sequence is followed by, optionally immediately followed by, a sgRNA backbone sequence of SEQ ID NOS: 115 and the tracrRNA comprises SEQ ID NO: 116, or
(b) the targeting sequence is followed by, optionally immediately followed by, a sgRNA backbone sequence of SEQ ID NOS: 117 and the tracrRNA comprises SEQ ID NO: 118;
(B) the gRNA is synthetic or recombinant; and/or
(C) the gRNA is a synthetic sgRNA and comprises at least one chemical modification, which is optionally:
(C-1) 2-O-methylation further optionally at first three and last three bases, and/or
(C-2) one or more 3′ phosphorothioate bonds, further optionally between first three and last two bases.

3. An isolated gRNA for CRISPR-mediated gene editing, wherein the gRNA comprises a targeting sequence comprising at least or consisting of 17 nucleotides, optionally 17, 18, 19, 20, 21, 22, 23, 24, 25, 26, 27, 28, 29, or 30 nucleotides, wherein:
(I) the targeting sequence comprises or consists of:
(i) (i-1) a sequence of at least 17 consecutive nucleotides, optionally 17, 18, 19, 20, 21, 22, 23, 24, 25, 26, 27, 28, 29, or 30 consequent nucleotides, contained in one or more of SEQ ID NO: 400, 401, 402, 405, 406, and/or 407, or (i-2) a sequence of:
(a) SEQ ID NO: 1, 410, 411, 412, or 413,
(b) SEQ ID NO: 2, 420, 421, 422, or 423,
(c) SEQ ID NO: 3, 430, 431, 432, or 433, or
(d) SEQ ID NO: 440, 441, 442, or 443,
(e) SEQ ID NO: 450, 451, 452, or 453,
(ii) a sequence of at least 17 nucleotides comprising one or more mutations, optionally one, two, three, four, or five mutations, said mutations relative to the at least 17 consecutive nucleotides, optionally 17, 18, 19, 20, 21, 22, 23, 24, 25, 26, 27, 28, 29, or 30 consequent nucleotides, of (i), optionally wherein the one or more mutations are at any nucleotide position(s) or are at position(s) other than the 4th to the 7th nucleotide positions from the 3′-end of the at least 17 consecutive nucleotides, or
(iii) a sequence of at least 17 nucleotides which comprises at least 85, 90, 95, 96, 97, 98 or 99% sequence identity to the sequence of at least 17 consecutive nucleotides, optionally 17, 18, 19, 20, 21, 22, 23, 24, 25, 26, 27, 28, 29, or 30 consecutive nucleotides, of (i),
optionally wherein the at least 17 consecutive nucleotides are;
(a) immediately upstream of a PAM or PFS of a Cas endonuclease in one or more of SEQ ID NO: 400, 401, 402, 405, 406, and/or 407, optionally wherein the Cas endonuclease is Cas9, further optionally wherein the Cas endonuclease is SpCas9 and/or the PAM sequence is 5′-NGG-3′ wherein N represents any nucleotide; or
(b) immediately downstream of the PAM or PFS of a Cas endonuclease in one or more of SEQ ID NO: 400, 401, 402, 405, 406, and/or 407, optionally wherein the Cas endonuclease is Cpf1 and/or the PAM sequence is 5′-TTTN-3′ wherein N represents any nucleotide; and/or
(II) when the gRNA is complexed with a Cas endonuclease, the targeting sequence guides the Cas endonuclease to allow for cleavage of exon 31 of the HTT gene or within 30 nucleotides upstream or downstream of the exon 31, optionally wherein:
(i) the cleavage is within one or more of SEQ ID NOS: 400, 401, 402, 405, 406, and/or 407, optionally at any one or more of the following positions:
(i-1) between the 36^{th} and 37^{th} nucleotides from the 5′ end of SEQ ID NO: 400, (i-2) between the 106^{th} and 107^{th} nucleotides from the 5′ end of SEQ ID NO: 400, (i-3) between the 185^{th} and 186^{th} nucleotides from the 5′ end of SEQ ID NO: 400, (i-4) between the 112^{th} and 113^{th} nucleotides from the 5′ end of SEQ ID NO: 400, (i-5) between the 101^{th} and 102^{th} nucleotides from the 5′ end of SEQ ID NO: 400, (i-6) between the 36^{th} and 37^{th} nucleotides from the 3′ end of SEQ ID NO: 405, (i-7) between the 106^{th} and 107^{th} nucleotides from the 3′ end of SEQ ID NO: 405, (i-8) between the 185^{th} and 186^{th} nucleotides from the 3′ end of SEQ ID NO: 405,
(i-9) between the 112^{th} and 113^{th} nucleotides from the 3′ end of SEQ ID NO: 405, and/or
(i-10) between the 101^{th} and 102^{th} nucleotides from the 3′ end of SEQ ID NO: 405; and/or
(ii) the Cas endonuclease is Cas 9, Cas3, Cas8a2, Cas8b, Cas8c, Cas10, Cas11, Cas12, Cas12a or Cpf1, Cas13, Cas13a, C2c1, C2c3, or C2c2, further optionally Cas9 or Cpf1, yet further optionally SpCas9,
optionally wherein the gRNA comprises one or more of the following features:
(A) (A-1) the gRNA is a sgRNA comprising in a single strand:
(i) a crRNA sequence comprising the targeting sequence and a crRNA backbone sequence, and
(ii) a tracrRNA sequence,
optionally wherein the crRNA sequence and the tracrRNA sequence are linked via a linker optionally comprising SEQ ID NO: 109, further optionally wherein the gRNA comprises the targeting sequence followed by, optionally immediately followed by, a sgRNA backbone sequence of any of SEQ ID NOS: 111-114, optionally wherein the sgRNA backbone sequence is followed by one or more uracils, further optionally 1-10 uracils, or
(A-2) the gRNA is a dgRNA formed by hybridization between:
(i) a crRNA comprising the targeting sequence and a crRNA backbone sequence, and
(ii) a tracrRNA,
optionally wherein:
(a) the targeting sequence is followed by, optionally immediately followed by, a sgRNA backbone sequence of SEQ ID NOS: 115 and the tracrRNA comprises SEQ ID NO: 116, or
(b) the targeting sequence is followed by, optionally immediately followed by, a sgRNA backbone sequence of SEQ ID NOS: 117 and the tracrRNA comprises SEQ ID NO: 118;
(B) the gRNA is synthetic or recombinant; and/or
(C) the gRNA is a synthetic sgRNA and comprises at least one chemical modification, which is optionally:
(C-1) 2′-O-methylation further optionally at first three and last three bases, and/or
(C-2) one or more 3′ phosphorothioate bonds, further optionally between first three and last two bases.

4. A combination of isolated gRNAs for effecting CRISPR gene editing of target sequences in at least 2 exons selected from exon 5, exon 8 and exon 31, wherein said combination of isolated gRNAs comprises at least 2 or all 3 of the following:
(i) at least one gRNA according to claim 1,
(ii) at least one gRNA according to claim 2, and
(iii) at least one gRNA according to claim 3.

5. A polynucleotide or polynucleotides encoding one or more of the isolated gRNAs of claim 1.

6. A polynucleotide or polynucleotides encoding one or more of the isolated gRNAs of claim 2.

7. A polynucleotide or polynucleotides encoding one or more of the isolated gRNAs of claim 3.

8. A polynucleotide or polynucleotides encoding the combination of isolated gRNAs according to claim 4.

9. A vector or vectors comprising the polynucleotide or polynucleotides of claim 5, 6, 7 or 8, operably linked to one or more regulatory sequences, optionally wherein the vector or vectors are individually selected from plasmids, RNA replicons, virus-like particles (VLPs), and viral vectors, optionally retroviral, lentiviral, or adenoviral vectors.

10. A ribonucleoprotein (RNP), which comprises:
(a) one or more isolated gRNAs of any one of claims 1-3; which is/are complexed with (b) a Cas endonuclease,
optionally wherein: the Cas endonuclease is:
(i) selected from the group consisting of Cas9, Cas3, Cas8a2, Cas8b, Cas8c, Cas10, Cas11, Cas12, Cas12a or Cpf1, Cas13, Cas13a, C2c1, C2c3, and C2c2;
(ii) a class 2 Cas endonuclease, optionally a type II, type V, or type VI Cas nuclease;
(iii) Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, eSpCas9, HypaCas9, FokI-Fused dCas9, xCas9, SpRY (variant of SpCas9), and SpG (variant of SpCas9); and/or
(iv) Cas9, optionally comprising any one of SEQ ID NOS: 600-611,
and optionally wherein the RNP is formed by mixing at an approximately equimolar ratio (I) a solution comprising the one or more isolated gRNAs, optionally wherein the pH of the solution is about 6 to 8, about 6.5 to 7.5, further optionally about 7, and (II) a solution comprising the Cas endonuclease, optionally wherein the pH of the solution is about 6 to 8, about 6.5 to 7.5, further optionally about 7, further optionally wherein the mixing is for about 5 minutes.

11. A composition comprising:
(A) a pharmaceutically acceptable carrier; and
(B) one or more RNPs according to claim 6; and
(C) optionally one or more template DNAs,
optionally wherein:
(I) the one or more RNP comprises one or more of (I-1)-(I-3):
(I-1) a first isolated gRNA according to claim 1, which comprises a first targeting sequence comprising or consisting of:
(i) a sequence of SEQ ID NO: 210, 211, 212, or 213,
(ii) a sequence comprising one or more mutations, optionally one, two, three, four, or five mutations, relative to SEQ ID NO: 210, 211, 212, or 213, optionally wherein the one or more mutations are at any nucleotide position(s) or are at position(s) other than the 4th to the 7th nucleotide positions from the 3′-end of SEQ ID NO: 210, 211, 212, or 213, or
(iii) a sequence comprising at least 85, 90, 95, 96, 97, 98 or 99% sequence identity to SEQ ID NO: 210, 211, 212, or 213;
(I-2) a second isolated gRNA according to claim 1, which comprises a second targeting sequence comprising or consisting of:
(i) a sequence of SEQ ID NO: 220, 221, 222, or 223,
(ii) a sequence comprising one or more mutations, optionally one, two, three, four, or five mutations, relative to SEQ ID NO: 220, 221, 222, or 223, optionally wherein the one or more mutations are at any nucleotide position(s) or are at position(s) other than the 4th to the 7th nucleotide positions from the 3′-end of SEQ ID NO: 220, 221, 222, or 223, or
(iii) a sequence comprising at least 85, 90, 95, 96, 97, 98 or 99% sequence identity to SEQ ID NO: 220, 221, 222, or 223; and/or
(I-3) a third isolated gRNA according to claim 1, which comprises a third targeting sequence comprising or consisting of:
(i) a sequence of SEQ ID NO: 230, 231, 232, or 233,
(ii) a sequence comprising one or more mutations, optionally one, two, three, four, or five mutations, relative to SEQ ID NO: 230, 231, 232, or 233, optionally wherein the one or more mutations are at any nucleotide position(s) or are at position(s) other than the 4th to the 7th nucleotide positions from the 3′-end of SEQ ID NO: 230, 231, 232, or 233, or
(iii) a sequence comprising at least 85, 90, 95, 96, 97, 98 or 99% sequence identity to SEQ ID NO: 230, 231, 232, or 233,
further optionally wherein the one or more RNP comprises all of:
(I-1) the first isolated gRNA which comprises the first targeting sequence of SEQ ID NO: 210, 211, 212, or 213;
(I-2) the second isolated gRNA comprising the second targeting sequence of SEQ ID NO: 220, 221, 222, or 223; and
(I-3) the third isolated gRNA comprising the third targeting sequence of SEQ ID NO: 230, 231, 232, or 233,
and optionally (I-4) one or more isolated gRNAs according to claim 1;
(II) the one or more RNP comprises one or more of (II-1)-(II-3):
(II-1) a fourth isolated gRNA according to claim 2, which comprises a fourth targeting sequence comprising or consisting of:
(i) a sequence of SEQ ID NO: 310, 311, 312, or 313,
(ii) a sequence comprising one or more mutations, optionally one, two, three, four, or five mutations, relative to SEQ ID NO: 310, 311, 312, or 313, optionally wherein the one or more mutations are at any nucleotide position(s) or are at position(s) other than the 4th to the 7th nucleotide positions from the 3′-end of SEQ ID NO: 310, 311, 312, or 313, or
(iii) a sequence comprising at least 85, 90, 95, 96, 97, 98 or 99% sequence identity to SEQ ID NO: 310, 311, 312, or 313;
(II-2) a fifth isolated gRNA according to claim 2, which comprises a fifth targeting sequence comprising or consisting of:
(i) a sequence of SEQ ID NO: 320, 321, 322, or 323,
(ii) a sequence comprising one or more mutations, optionally one, two, three, four, or five mutations, relative to SEQ ID NO: 320, 321, 322, or 323, optionally wherein the one or more mutations are at any nucleotide position(s) or are at position(s) other than the 4th to the 7th nucleotide positions from the 3′-end of SEQ ID NO: 320, 321, 322, or 323, or
(iii) a sequence comprising at least 85, 90, 95, 96, 97, 98 or 99% sequence identity to SEQ ID NO: 320, 321, 322, or 323; and/or
(II-3) a sixth isolated gRNA according to claim 2, which comprises a sixth targeting sequence comprising or consisting of:
(i) a sequence of SEQ ID NO: 330, 331, 332, or 333,
(ii) a sequence comprising one or more mutations, optionally one, two, three, four, or five mutations, relative to SEQ ID NO: 330, 331, 332, or 333, optionally wherein the one or more mutations are at any nucleotide position(s) or are at position(s) other than the 4th to the 7th nucleotide positions from the 3′-end of SEQ ID NO: 330, 331, 332, or 333, or
(iii) a sequence comprising at least 85, 90, 95, 96, 97, 98 or 99% sequence identity to SEQ ID NO: 330, 331, 332, or 333,
further optionally wherein the one or more RNP comprises all of:
(II-1) the fourth isolated gRNA which comprises the fourth targeting sequence of SEQ ID NO: 310, 311, 312, or 313;
(II-2) the fifth isolated gRNA comprising the fifth targeting sequence of SEQ ID NO:
320, 321, 322, or 323; and
(II-3) the sixth isolated gRNA comprising the sixth targeting sequence of SEQ ID NO: 330, 331, 332, or 333,
and optionally (II-4) one or more isolated gRNAs according to claim 2; and/or
(III) the one or more RNP comprises one or more of (III-1)-(III-4):
(III-1) a seventh isolated gRNA according to claim 3, which comprises a seventh targeting sequence comprising or consisting of:
(i) a sequence of SEQ ID NO: 410, 411, 412, or 413,
(ii) a sequence comprising one or more mutations, optionally one, two, three, four, or five mutations, relative to SEQ ID NO: 410, 411, 412, or 413, optionally wherein the one or more mutations are at any nucleotide position(s) or are at position(s) other than the 4th to the 7th nucleotide positions from the 3′-end of 410, 411, 412, or 413, or
(iii) a sequence comprising at least 85, 90, 95, 96, 97, 98 or 99% sequence identity to SEQ ID NO: 410, 411, 412, or 413;
(III-2) an eighth isolated gRNA according to claim 3, which comprises an eighth targeting sequence comprising or consisting of:
(i) a sequence of SEQ ID NO: 420, 421, 422, or 423,
(ii) a sequence comprising one or more mutations, optionally one, two, three, four, or five mutations, relative to SEQ ID NO: 420, 421, 422, or 423, optionally wherein the one or more mutations are at any nucleotide position(s) or are at position(s) other than the 4th to the 7th nucleotide positions from the 3′-end of SEQ ID NO: 420, 421, 422, or 423, or
(iii) a sequence comprising at least 85, 90, 95, 96, 97, 98 or 99% sequence identity to SEQ ID NO: 420, 421, 422, or 423;
(III-3) a ninth isolated gRNA according to claim 3, which comprises a ninth targeting sequence comprising or consisting of:
(i) a sequence of SEQ ID NO: 430, 431, 432, or 433,
(ii) a sequence comprising one or more mutations, optionally one, two, three, four, or five mutations, relative to SEQ ID NO: 430, 431, 432, or 433, optionally wherein the one or more mutations are at any nucleotide position(s) or are at position(s) other than the 4th to the 7th nucleotide positions from the 3′-end of SEQ ID NO: 430, 431, 432, or 433, or
(iii) a sequence comprising at least 85, 90, 95, 96, 97, 98 or 99% sequence identity to SEQ ID NO: 430, 431, 432, or 433; and/or
(III-4) a tenth isolated gRNA according to claim 3, which comprises a tenth targeting sequence comprising or consisting of:
(i) a sequence of SEQ ID NO: 440, 441, 442, or 443 or 450, 451, 452, or 453,
(ii) a sequence comprising one or more mutations, optionally one, two, three, four, or five mutations, relative to SEQ ID NO: 440, 441, 442, or 443 or 450, 451, 452, or 453, optionally wherein the one or more mutations are at any nucleotide position(s) or are at position(s) other than the 4th to the 7th nucleotide positions from the 3′-end of SEQ ID NO: 440, 441, 442, or 443 or 450, 451, 452, or 453, or
(iii) a sequence comprising at least 85, 90, 95, 96, 97, 98 or 99% sequence identity to SEQ ID NO: 440, 441, 442, or 443 or 450, 451, 452, or 453,
further optionally wherein the one or more RNP comprises:
all of (III-1) the seventh isolated gRNA which comprises the seventh targeting sequence of SEQ ID NO: 410, 411, 412, or 413,
(III-2) the eighth isolated gRNA comprising the eighth targeting sequence of SEQ ID NO: 420, 421, 422, or 423, and
(III-3) the ninth isolated gRNA comprising the ninth targeting sequence of SEQ ID NO: 430, 431, 432, or 433,
and optionally (III-5) one or more isolated gRNAs according to claim 3; or
all of (III-1) the seventh isolated gRNA which comprises the seventh targeting sequence of SEQ ID NO: 410, 411, 412, or 413,
(III-3) the ninth isolated gRNA comprising the ninth targeting sequence of SEQ ID NO: 430, 431, 432, or 433, and
(III-4) the tenth isolated gRNA comprising the tenth targeting sequence of SEQ ID NO: 440, 441, 442, or 443 or 450, 451, 452, or 453,
and optionally (III-5) one or more isolated gRNAs according to claim 3, and optionally wherein the pharmaceutically acceptable carrier comprises a lipid-based transfection competent vesicle (TCV).

12. The composition of claim 11, wherein:
(a) the pharmaceutically acceptable carrier comprises a lipid-based TCV, and
(b) the one or more template DNAs, if present, and/or the one or more RNPs are encapsulated in the TCV, optionally by:
(i) providing an aqueous solution comprising the TCV, optionally wherein the aqueous solution:
(i-1) has the pH of about 3 to about 8, about 4 to about 7.5, about 3.5 to 4.5, or about 4, optionally wherein said aqueous solution comprises an acetate buffer, optionally an about 25-100 mM or about 25 mM acetate buffer; and/or
(i-2) is: substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS); optionally substantially, essentially, or entirely free of organic solvents and/or detergents; further optionally substantially, essentially, or entirely free of destabilizing agents; and
(ii) mixing the one or more template DNAs, if present, and/or one or more of the one or more RNPs with the aqueous solution, optionally wherein:
(ii-1) the mixing comprises gentle mixing (optionally repeated manual reciprocation of the TCV-generating fluid in a pipette), mixing using a staggered herringbone micromixer (SHM), T-junction mixing, or extrusion, and optionally wherein the mixing time is about 0.1 second to about 20 minutes;
(ii-2) the mixing is performed: substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS); optionally substantially, essentially, or entirely free of organic solvents and/or detergents; further optionally substantially, essentially, or entirely free of destabilizing agents;
(iii-3) when more than one RNPs are encapsulated in the TCV, the mixing comprises mixing an equimolar ratio of the more than one RNPs with the aqueous solution; and/or
(iii-4) in the mixing step, the lipid-based TCV and the RNP(s) are mixed at an about 100:1 to about 10000:1 molar ratio, optionally at about 500:1 to about 5000:1 molar ratio, further optionally at about 1000:1 to about 2000:1 molar ratio, of the total lipid components of the lipid-based TCV: the RNP(s),
optionally wherein the one or more template DNAs, if present, is/are co-encapsulated with or separately encapsulated from one or more of the one or more RNPs.

13. A composition comprising:
(A) a pharmaceutically acceptable carrier; and
(B) (a) one or more isolated gRNAs according to any one of claims 1-4 or one or more polynucleotides encoding the one or more isolated gRNAs, and
(b) a Cas endonuclease or a polynucleotide encoding a Cas endonuclease; and
(C) optionally one or more template DNAs or one or more polynucleotides encoding one or more template DNAs,
optionally wherein:
(I) the one or more isolated gRNAs comprises one or more of (I-1)-(I-3):
(I-1) a first isolated gRNA according to claim 1, which comprises a first targeting sequence comprising or consisting of:
(i) a sequence of SEQ ID NO: 210, 211, 212, or 213,
(ii) a sequence comprising one or more mutations, optionally one, two, three, four, or five mutations, relative to SEQ ID NO: 210, 211, 212, or 213, optionally wherein the one or more mutations are at any nucleotide position(s) or are at position(s) other than the 4th to the 7th nucleotide positions from the 3′-end of SEQ ID NO: 210, 211, 212, or 213, or
(iii) a sequence comprising at least 85, 90, 95, 96, 97, 98 or 99% sequence identity to SEQ ID NO: 210, 211, 212, or 213;
(I-2) a second isolated gRNA according to claim 1, which comprises a second targeting sequence comprising or consisting of:
(i) a sequence of SEQ ID NO: 220, 221, 222, or 223,
(ii) a sequence comprising one or more mutations, optionally one, two, three, four, or five mutations, relative to SEQ ID NO: 220, 221, 222, or 223, optionally wherein the one or more mutations are at any nucleotide position(s) or are at position(s) other than the 4th to the 7th nucleotide positions from the 3′-end of SEQ ID NO: 220, 221, 222, or 223, or
(iii) a sequence comprising at least 85, 90, 95, 96, 97, 98 or 99% sequence identity to SEQ ID NO: 220, 221, 222, or 223; and/or
(I-3) a third isolated gRNA according to claim 1, which comprises a third targeting sequence comprising or consisting of:
(i) a sequence of SEQ ID NO: 230, 231, 232, or 233,
(ii) a sequence comprising one or more mutations, optionally one, two, three, four, or five mutations, relative to SEQ ID NO: 230, 231, 232, or 233, optionally wherein the one or more mutations are at any nucleotide position(s) or are at position(s) other than the 4th to the 7th nucleotide positions from the 3′-end of SEQ ID NO: 230, 231, 232, or 233, or
(iii) a sequence comprising at least 85, 90, 95, 96, 97, 98 or 99% sequence identity to SEQ ID NO: 230, 231, 232, or 233,
further optionally wherein the one or more isolated gRNAs comprises all of:
(I-1) the first isolated gRNA which comprises the first targeting sequence of SEQ ID NO: 210, 211, 212, or 213;
(I-2) the second isolated gRNA comprising the second targeting sequence of SEQ ID NO: 220, 221, 222, or 223; and
(I-3) the third isolated gRNA comprising the third targeting sequence of SEQ ID NO: 230, 231, 232, or 233,
and optionally (I-4) one or more isolated gRNAs according to claim 1;
(II) the one or more isolated gRNAs comprises one or more of (II-1)-(II-3):
(II-1) a fourth isolated gRNA according to claim 2, which comprises a fourth targeting sequence comprising or consisting of:
(i) a sequence of SEQ ID NO: 310, 311, 312, or 313,
(ii) a sequence comprising one or more mutations, optionally one, two, three, four, or five mutations, relative to SEQ ID NO: 310, 311, 312, or 313, optionally wherein the one or more mutations are at any nucleotide position(s) or are at position(s) other than the 4th to the 7th nucleotide positions from the 3′-end of SEQ ID NO: 310, 311, 312, or 313, or
(iii) a sequence comprising at least 85, 90, 95, 96, 97, 98 or 99% sequence identity to SEQ ID NO: 310, 311, 312, or 313;
(II-2) a fifth isolated gRNA according to claim 2, which comprises a fifth targeting sequence comprising or consisting of:
(i) a sequence of SEQ ID NO: 320, 321, 322, or 323,
(ii) a sequence comprising one or more mutations, optionally one, two, three, four, or five mutations, relative to SEQ ID NO: 320, 321, 322, or 323, optionally wherein the one or more mutations are at any nucleotide position(s) or are at position(s) other than the 4th to the 7th nucleotide positions from the 3′-end of SEQ ID NO: 320, 321, 322, or 323, or
(iii) a sequence comprising at least 85, 90, 95, 96, 97, 98 or 99% sequence identity to SEQ ID NO: 320, 321, 322, or 323; and/or
(II-3) a sixth isolated gRNA according to claim 2, which comprises a sixth targeting sequence comprising or consisting of:
(i) a sequence of SEQ ID NO: 330, 331, 332, or 333,
(ii) a sequence comprising one or more mutations, optionally one, two, three, four, or five mutations, relative to SEQ ID NO: 330, 331, 332, or 333, optionally wherein the one or more mutations are at any nucleotide position(s) or are at position(s) other than the 4th to the 7th nucleotide positions from the 3′-end of SEQ ID NO: 330, 331, 332, or 333, or
(iii) a sequence comprising at least 85, 90, 95, 96, 97, 98 or 99% sequence identity to SEQ ID NO: 330, 331, 332, or 333,
further optionally wherein the one or more isolated gRNAs comprises all of:
(II-1) the first isolated gRNA which comprises the first targeting sequence of SEQ ID NO: 310, 311, 312, or 313;
(II-2) the second isolated gRNA comprising the second targeting sequence of SEQ ID NO: 320, 321, 322, or 323; and
(II-3) the third isolated gRNA comprising the third targeting sequence of SEQ ID NO: 330, 331, 332, or 333,
and optionally (II-4) one or more isolated gRNAs according to claim 2; and/or
(III) the one or more isolated gRNAs comprises one or more of (III-1)-(III-3):
(III-1) a seventh isolated gRNA according to claim 3, which comprises a seventh targeting sequence comprising or consisting of:
(i) a sequence of SEQ ID NO: 410, 411, 412, or 413,
(ii) a sequence comprising one or more mutations, optionally one, two, three, four, or five mutations, relative to SEQ ID NO: 410, 411, 412, or 413, optionally wherein the one or more mutations are at any nucleotide position(s) or are at position(s) other than the 4th to the 7th nucleotide positions from the 3′-end of 410, 411, 412, or 413, or
(iii) a sequence comprising at least 85, 90, 95, 96, 97, 98 or 99% sequence identity to SEQ ID NO: 410, 411, 412, or 413;
(III-2) an eighth isolated gRNA according to claim 3, which comprises an eighth targeting sequence comprising or consisting of:
(i) a sequence of SEQ ID NO: 420, 421, 422, or 423,
(ii) a sequence comprising one or more mutations, optionally one, two, three, four, or five mutations, relative to SEQ ID NO: 420, 421, 422, or 423, optionally wherein the one or more mutations are at any nucleotide position(s) or are at position(s) other than the 4th to the 7th nucleotide positions from the 3′-end of SEQ ID NO: 420, 421, 422, or 423, or
(iii) a sequence comprising at least 85, 90, 95, 96, 97, 98 or 99% sequence identity to SEQ ID NO: 420, 421, 422, or 423;
(III-3) a ninth isolated gRNA according to claim 3, which comprises a ninth targeting sequence comprising or consisting of:
(i) a sequence of SEQ ID NO: 430, 431, 432, or 433,
(ii) a sequence comprising one or more mutations, optionally one, two, three, four, or five mutations, relative to SEQ ID NO: 430, 431, 432, or 433, optionally wherein the one or more mutations are at any nucleotide position(s) or are at position(s) other than the 4th to the 7th nucleotide positions from the 3′-end of SEQ ID NO: 430, 431, 432, or 433, or
(iii) a sequence comprising at least 85, 90, 95, 96, 97, 98 or 99% sequence identity to SEQ ID NO: 430, 431, 432, or 433; and/or
(III-4) a tenth isolated gRNA according to claim 3, which comprises a tenth targeting sequence comprising or consisting of:
(i) a sequence of SEQ ID NO: 440, 441, 442, or 443 or 450, 451, 452, or 453,
(ii) a sequence comprising one or more mutations, optionally one, two, three, four, or five mutations, relative to SEQ ID NO: 440, 441, 442, or 443 or 450, 451, 452, or 453, optionally wherein the one or more mutations are at any nucleotide position(s) or are at position(s) other than the 4th to the 7th nucleotide positions from the 3′-end of SEQ ID NO: 440, 441, 442, or 443 or 450, 451, 452, or 453, or
(iii) a sequence comprising at least 85, 90, 95, 96, 97, 98 or 99% sequence identity to SEQ ID NO: 440, 441, 442, or 443 or 450, 451, 452, or 453,
further optionally wherein the one or more isolated gRNAs comprises:
all of (III-1) the seventh isolated gRNA which comprises the seventh targeting sequence of SEQ ID NO: 410, 411, 412, or 413,
(III-2) the eighth isolated gRNA comprising the eighth targeting sequence of SEQ ID NO: 420, 421, 422, or 423, and
(III-3) the ninth isolated gRNA comprising the ninth targeting sequence of SEQ ID NO: 430, 431, 432, or 433,
and optionally (III-5) one or more isolated gRNAs according to claim 3; or
all of (III-1) the seventh isolated gRNA which comprises the seventh targeting sequence of SEQ ID NO: 410, 411, 412, or 413,
(III-3) the ninth isolated gRNA comprising the ninth targeting sequence of SEQ ID NO: 430, 431, 432, or 433, and
(III-4) the tenth isolated gRNA comprising the tenth targeting sequence of SEQ ID NO: 440, 441, 442, or 443 or 450, 451, 452, or 453,
and optionally (III-5) one or more isolated gRNAs according to claim 3,
and optionally wherein the Cas endonuclease is:
(i) selected from the group consisting of Cas9, Cas3, Cas8a2, Cas8b, Cas8c, Cas10, Cas11, Cas12, Cas12a or Cpf1, Cas13, Cas13a, C2c1, C2c3, and C2c2;
(ii) a class 2 Cas endonuclease, optionally a type II, type V, or type VI Cas nuclease;
(iii) Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, eSpCas9, HypaCas9, FokI-Fused dCas9, xCas9, SpRY (variant of SpCas9), and SpG (variant of SpCas9), and/or
(iv) Cas9, optionally comprising any one of SEQ ID NOS: 600-611,
and optionally wherein the pharmaceutically acceptable carrier comprises a lipid-based TCV.

14. The composition of claim 13, wherein in (B) the composition comprises:
(I) (a) the one or more isolated gRNAs, and
(b) the Cas endonuclease;
(II) (a) the one or more isolated gRNAs, and
(b) a vector comprising the polynucleotide encoding a Cas endonuclease;
(III) (a) one or more vectors comprising the one or more polynucleotides encoding the one or more isolated gRNAs, and
(b) the Cas endonuclease; or
(IV) (a) one or more vectors comprising the one or more polynucleotides encoding the one or more isolated gRNAs, and
(b) a vector comprising the polynucleotide encoding a Cas endonuclease,
optionally wherein the composition comprises one or more of the following features:
(i) the one or more vectors of (III)(a) and (IV)(a) and the vector of (II)(b) and (IV)(b) are individually selected from plasmids, RNA replicons, virus-like particles (VLPs), and viral vectors, optionally retroviral, lentiviral, or adenoviral vectors;
(ii) in (III)(a) and/or (IV)(a), when the one or more isolated gRNAs are more than one isolated gRNAs, the more than one gRNAs are encoded in a single vector or in separate vectors; and/or
(iii) in (IV), the one or more isolated gRNAs and the Cas endonuclease may be encoded in a single vector or in separate vectors.

15. The composition of any one of claims 11-14, wherein the one or more template DNAs comprise(s):
(A) one or more single-strand oligo DNA nucleotide molecules (ssODNs), each comprising or consisting of a 5′ homology arm, an optional central region, and a 3′ homology arm, wherein:
(A-I) (a) (i) the 5′ homology arm comprises or consists of (i-1) the sequence corresponding to the first nucleotide to at least the 10th nucleotide counting from the 3′-end of SEQ ID NO: 268, (i-2) the sequence of any of SEQ ID NOS: 261-268, or (i-3) a sequence comprising at least one mutation, optionally one, two, three, four, five, six, seven, eight, nine, or ten mutation(s), relative to the sequence of (i-1) or (i-2),
(ii) the optional central region is 1-100 nucleotides (nt) in length; and
(iii) the 3′ homology arm comprises or consists of (iii-1) the sequence corresponding to the first nucleotide to at least the 10th nucleotide counting from the 5′-end of SEQ ID NO: 278, (iii-2) the sequence of any of SEQ ID NOS: 271-278, or (iii-3) a sequence comprising at least one mutation, optionally one, two, three, four, five, six, seven, eight, nine, or ten mutation(s), relative to the sequence of (iii-1) or (iii-2),
optionally wherein the ssODN comprises or consists of the sequence of any of SEQ ID NOs: 281-288;
(b) the sequence of the ssODN is fully complementary to the sequence any of the ssODNs of (a), optionally wherein the ssODN comprises or consists of the sequence of any of SEQ ID NOs: 291-298;
(A-II) (a) (i) the 5′ homology arm comprises or consists of (i-1) the sequence corresponding to the first nucleotide to at least the 10th nucleotide counting from the 3′-end of SEQ ID NO: 368, (i-2) the sequence of any of SEQ ID NOS: 361-368, or (i-3) a sequence comprising at least one mutation, optionally one, two, three, four, five, six, seven, eight, nine, or ten mutation(s), relative to the sequence of (i-1) or (i-2),
(ii) the optional central region is 0-100 nucleotides (nt) in length; and
(iii) the 3′ homology arm comprises or consists of (iii-1) the sequence corresponding to the first nucleotide to at least the 10th nucleotide counting from the 5′-end of SEQ ID NO: 378, (iii-2) the sequence of any of SEQ ID NOS: 371-378, or (iii-3) a sequence comprising at least one mutation, optionally one, two, three, four, five, six, seven, eight, nine, or ten mutation(s), relative to the sequence of (iii-1) or (iii-2),
optionally wherein the ssODN comprises or consists of the sequence of any of SEQ ID NOs: 381-388;
(b) the sequence of the ssODN is fully complementary to the sequence any of the ssODNs of (a), optionally wherein the ssODN comprises or consists of the sequence of any of SEQ ID NOs: 391-388; or
(A-III) (a) (i) the 5′ homology arm comprises or consists of (i-1) the sequence corresponding to the first nucleotide to at least the 10th nucleotide counting from the 3′-end of SEQ ID NO: 468, (i-2) the sequence of any of SEQ ID NOS: 461-468, or (i-3) a sequence comprising at least one mutation, optionally one, two, three, four, five, six, seven, eight, nine, or ten mutation(s), relative to the sequence of (i-1) or (i-2),
(ii) the optional central region is 0-100 nucleotides (nt) in length; and
(iii) the 3′ homology arm comprises or consists of (iii-1) the sequence corresponding to the first nucleotide to at least the 10th nucleotide counting from the 5′-end of SEQ ID NO: 478, (iii-2) the sequence of any of SEQ ID NOS: 471-478, or (iii-3) a sequence comprising at least one mutation, optionally one, two, three, four, five, six, seven, eight, nine, or ten mutation(s), relative to the sequence of (iii-1) or (iii-2),
optionally wherein the ssODN comprises or consists of the sequence of any of SEQ ID NOs: 481-488;
(b) the sequence of the ssODN is fully complementary to the sequence any of the ssODNs of (a), optionally wherein the ssODN comprises or consists of the sequence of any of SEQ ID NOs: 491-488; and/or
(B) one or more double-strand DNA molecules, which comprise:
(B-I) a first strand comprising any of the ssODN sequences of (A-I) and a second strand complementary to the first strand;
(B-II) a first strand comprising any of the ssODN sequences of (A-II) and a second strand complementary to the first strand; or
(B-III) a first strand comprising any of the ssODN sequences of (A-III) and a second strand complementary to the first strand.

16. The composition of any one of claims 11-15, wherein the TCV comprises at least one cationic or ionizable cationic lipid, optionally wherein the TCV comprises one or more of the following features:
(i) the at least one cationic or ionizable cationic lipid comprises, essentially consists of, or consists of a lipid selected from the group consisting of N,N-dimethyl-2,3-dioleyloxy)propylamine (DODMA), 1,2-dioleoyl-3-dimethylammonium propane (“DODAP”), 1,2-Dilinoleoyl-3-dimethylaminopropane (DLinDAP), N,N-dimethyl-2,2-di-(9Z,12Z)-9,12-octadecadien-1-yl-1,3-dioxolane-4-ethanamine (KC2), (6Z,9Z,28Z,31Z)-heptatriaconta-6,9,28,31-tetraen-19-yl 4-(dimethylamino)butanoate (MC3), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxyl)propyl)-N,N,N-trimethylammonium chloride (DOTMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAR.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLin-K-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3 aH-cyclopenta[d][1,3]dioxol-5-amine (ALNY-100), N-(2,3-dioleyloxyl)propyl-N,N-N-triethylammonium chloride (“DOTMA”); 1,2-Dioleyloxy-3-trimethylaminopropane chloride salt (“DOTAP.Cl”); 3.beta.-(N—(N′,N′-dimethylaminoethane)-carbamoyl)cholesterol (“DC-Chol”), N-(1-(2,3-dioleyloxyl)propyl)-N-2-(sperminecarboxamido)ethyl)-N,N-dimethyl-ammonium trifluoroacetate (“DOSPA”), dioctadecylamidoglycyl carboxyspermine (“DOGS”), and N-(1,2-dimyristyloxyprop-3-yl)-N,N-dimethyl-N-hydroxyethyl ammonium bromide (“DMRIE”), and any combinations thereof;
(ii) the TCV further comprises at least one helper lipid, optionally wherein the at least one helper lipid comprises, essentially consists of, or consists of a lipid selected from the group consisting of dioleoylphosphatidylethanolamine (DOPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), and any combinations thereof,
(iii) the TCV further comprises at least one phospholipid, optionally wherein the at least one phospholipid comprises, essentially consists of, or consists of a lipid selected from the group consisting of distearoylphosphatidylcholine (DSPC), dioleoyl phosphatidylethanolamine (DOPE), dipalmitoylphosphatidylcholine (DPPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and any combinations thereof,
(iv) the TCV further comprises at least one cholesterol or cholesterol derivative, optionally wherein the at least one cholesterol or cholesterol derivative comprises, essentially consists of, or consists of a cholesterol or cholesterol derivative selected from the group consisting of cholesterol, N,N-dimethyl-N-ethylcarboxamidocholesterol (DC-Chol), 1,4-bis(3-N-oleylamino-propyl)piperazine, imidazole cholesterol ester (ICE), and any combinations thereof;
(v) the TCV further comprises at least one PEG or PEG-lipid, optionally wherein the at least one PEG-lipid comprises, essentially consists of, or consists of a PEG-lipid selected from the group consisting of PEG-myristoyl diglyceride (PEG-DMG) (e.g., 1,2-dimyristoyl-rac-glycero-3-methoxypolyethylene glycol-2000 (Avanti® Polar Lipids (Birmingham, AL)), which is a mixture of 1,2-DMG PEG2000 and 1,3-DMG PEG2000 (e.g., in about 97:3 ratio)), PEG-phosphatidylethanolamine and phosphatidic acid, PEG-ceramide conjugates (e.g., PEG-CerC14 or PEG-CerC20), PEG-modified dialkylamines, PEG-modified 1,2-diacyloxypropan-3-amines, and any combinations thereof,
(vi) the TCV is: substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS); optionally substantially, essentially, or entirely free of organic solvents and/or detergents; further optionally substantially, essentially, or entirely free of destabilizing agents, and/or
(vii) the TCV is substantially, essentially, or entirely free of any permanently cationic lipids and/or any permanently anionic lipids;
or any combination of (i) to (vii),
further optionally wherein the TCV is formed by:
(a) providing a first solution comprising all components of the TCV, optionally at about 20-35 mM, in ethanol;
(b) providing a second solution, which is aqueous and contains acetate and/or citrate, optionally sodium acetate and/or sodium citrate, optionally at about 25 mM, optionally wherein the pH of the solution is about 3.5 to 4.5 or about 4;
(c) combining the first and second solutions by gentle mixing (optionally repeated manual reciprocation of the TCV-generating fluid in a pipette), mixing using a staggered herringbone micromixer (SHM), T-junction mixing, or extrusion; and
(d) removing ethanol, optionally by dialysis or evaporation,
further optionally wherein:
(a) the size of the TCV before encapsulation is in a range of about 9 nm to about 80 nm, optionally about 10-40 nm, further optionally about 20-35 nm, at pH of about 3.5 to 4.5 or about 4;
(b) the size of the TCV after encapsulation of the at least one cargo is in a range of about 80 nm to about 1500 nm, optionally about 800 nm to about 1400 or about 1000 nm to about 1200 nm or about 80 nm to about 300 nm or about 100 nm to about 250 nm;
(c) the TCV after encapsulation is further comprised in a matrix vesicle, which is optionally for gradual release of the TCV; and/or
(d) the final ethanol concentration of the composition is 5% (v/v) or below, 2.5% (v/v) or below, 1.5% (v/v) or below, and preferably 0.5% (v/v) or below.

17. The composition of claim 16, wherein:
(a) the amount of the at least one cationic or ionizable cationic lipid relative to the total components of the TCV is: (a-1) about 10 mol % to about 70 mol %, about 10 mol % to about 60 mol %, about 10 mol % to about 50 mol %, about 10 mol % to about 40 mol %, about 10 mol % to about 30 mol %, about 15 mol % to about 25 mol %, about 18 mol % to about 22 mol %, about 19 mol % to about 21 mol %, about 19.5 mol % to about 20.5 mol %, about 19.8 mol % to about 20.2 mol %, or about 20 mol %; or (a-2) about 10 mol % to about 70 mol %, about 20 mol % to about 70 mol %, about 30 mol % to about 70 mol %, about 40 mol % to about 70 mol %, about 40 mol % to about 60 mol %, about 45 mol % to about 55 mol %, about 48 mol % to about 52 mol %, about 49 mol % to about 51 mol %, about 49.5 mol % to about 50.5 mol %, about 49.8 mol % to about 50.2 mol %, or about 50 mol %;
(b) in (ii), the amount of the at least one helper lipid relative to the total components of the TCV is about 10 mol % to about 60 mol %, about 10 mol % to about 50 mol %, about 10 mol % to about 40 mol %, about 20 mol % to about 40 mol %, about 25 mol % to about 35 mol %, about 28 mol % to about 32 mol %, about 29 mol % to about 31 mol %, about 29.5 mol % to about 30.5 mol %, about 29.8 mol % to about 30.2 mol %, or about 30 mol %;
(c) in (iii), the amount of the at least one phospholipid relative to the total components of the TCV is about 5 mol % to about 65 mol %, about 5 mol % to about 55 mol %, about 5 mol % to about 45 mol %, about 5 mol % to about 35 mol %, about 5 mol % to about 25 mol %, about 5 mol % to about 15 mol %, about 8 mol % to about 12 mol %, about 9 mol % to about 11 mol %, about 9.5 mol % to about 10.5 mol %, about 9.8 mol % to about 10.2 mol %, or about 10 mol %;
(d) in (iv), the amount of the at least one cholesterol or cholesterol derivative relative to the total components of the TCV is about 20 mol % to about 60 mol %, about 25 mol % to about 55 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %, or about 39%; and/or
(e) in (v), the amount of the at least one PEG or PEG-lipid relative to the total components of the TCV is about 0.1 mol % to about 5 mol %, 0.1 mol % to about 4 mol %, 0.1 mol % to about 3 mol %, 0.1 mol % to about 2 mol %, 0.5 mol % to about 1.5 mol %, 0.8 mol % to about 1.2 mol %, 0.9 mol % to about 1.1 mol %, or about 1 mol %.

18. The composition of any one of claims 11-17, wherein:
(I) the TCV comprises, essentially consists of, or consists of:
(i) at least one ionizable cationic lipid, which is optionally DODMA;
(ii) at least one helper lipid, which is optionally DOPE;
(iii) at least one phospholipid, which is optionally DSPC; and
(iv) at least one cholesterol or cholesterol derivative,
optionally wherein the amounts of the at least one ionizable cationic lipid, the at least one helper lipid, the at least one phospholipid, and the at least one cholesterol or cholesterol derivative, relative to the total components of the TCV, is about 20 mol %, about 30 mol %, about 10 mol %, and about 40 mol %, respectively; or
(II) the TCV comprises, essentially consists of, or consists of:
(i) at least one ionizable cationic lipid, which is optionally DODMA;
(ii) at least one helper lipid, which is optionally DOPE;
(iii) at least one phospholipid, which is optionally DSPC;
(iv) at least one cholesterol or cholesterol derivative; and
(v) at least one PEG or PEG-lipid, which is optionally PEG-DMG,
optionally wherein the amounts of the at least one ionizable cationic lipid, the at least one helper lipid, the at least one phospholipid, the at least one cholesterol or cholesterol derivative, and the at least one PEG or PEG-lipid, relative to the total components of the TCV, is about 20 mol %, about 30 mol %, about 10 mol %, about 39 mol %, and about 1 mol %, respectively,
and wherein the TCV is: substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS),
optionally wherein the TCV is:
(A) substantially, essentially, or entirely free of organic solvents and/or detergents;
(B) substantially, essentially, or entirely free of destabilizing agents;
(C) substantially, essentially, or entirely free of any permanently cationic lipids and/or any permanently anionic lipids;
and/or
(D) stable for prolonged periods of time at about 1 to about 40° C., about 5 to about 35° C., about 10 to about 30° C., or about 15 to about 25° C.,
and further optionally wherein the TCV or the composition further comprises and/or is stored in the presence of at least one cryoprotectant, optionally wherein:
(a) the cryoprotectant comprises a sugar-based molecule, which is optionally sucrose, trehalose, or a combination thereof;
(b) the concentration of the cryoprotectant is about 1% to about 40%, about 3% to about 30%, about 5% to about 30%, about 10% to about 20%, or about 15%;
(c) the TCV is stable at a freezing temperature, optionally at about −20° C. or about −80° C., optionally for at least about one week, at least about two weeks, at least about three weeks, at least about a month, at least about two months, at least about four months, at least about five months, at least about six months, at least about nine months, at least about a year, or at least about two years, or longer, further optionally for about one week to about two year, about two weeks to about a year, about three weeks to about nine month, about one to about six months, about one to five months, about one to four months, about one to three months, or about one to two months; or
(d) any combination of (a)-(c).

19. The composition of any one of claims 11-18, further comprising one or more agents for preventing, ameliorating, slowing the progression of, and/or treating Huntington's disease (HD), which optionally comprise(s):
(a) a drug for controlling movements, optionally tetrabenazine, deutetrabenazine, haloperidol, fluphenazine, risperidone, olanzapine, quetiapine, amantadine, levetiracetam, and/or clonazepam; and/or
(b) a drug for controlling psychiatric disorders, optionally (i) an antidepressant, optionally citalopram, escitalopram, fluoxetine, and/or sertraline, (ii) an antipsychotic drug, optionally quetiapine, risperidone, and olanzapine, and/or (iii) a mood-stabilizing drug, optionally divalproex, carbamazepine, and/or lamotrigine.

20. A method of effecting CRISPR-mediated gene editing of the HTT gene in one or more target cells and/or one or more target tissues, the method comprising applying the composition of any one of claims 11-19 to the one or more cells or one or more tissues, optionally wherein the one or more target cells comprise one or more of the following:
(i) a mammalian cell, a human cell, a cell line, a stem cell-derived cell, an induced pluripotent stem cell (iPSC)-derived cell, a primary cell, and/or a cell derived from a subject who has or has a risk of developing HD;
(ii) a brain cell, optionally a neural cell, a cortical neuron, a cell of the basal ganglia, a cell of the striatum, a cell of the caudate nucleus, and/or a cell of the putamen,
(iii) a myocyte, optionally a skeletal myocyte, a cardiomyocyte, and/or a smooth myocyte;
(iv) a cell of an endocrinal system, optionally a pancreatic cell and/or an adipocyte;
(v) a blood cell, optionally a lymphocyte, a macrophage, and/or a monocyte; and/or
(vi) a fibroblast and/or a lymphoblast;
and optionally wherein the one or more target tissues comprise one or more of the following:
(vii) a mammalian tissue, a human tissue, a primary tissue, and/or a tissue from a subject who has or has a risk of developing HD;
(viii) a brain tissue, optionally a nervous tissue, a tissue of the basal ganglia, a tissue of the striatum, a tissue of the caudate nucleus, and/or a tissue of the putamen;
(ix) a muscle tissue, optionally a skeletal muscle tissue, a cardiac tissue, and/or a smooth muscle tissue;
(x) an endocrine tissue, optionally a pancreatic tissue and/or an adipose tissue; and/or
(xi) a lymphoid and/or myeloid tissue, optionally a tissue of the bone marrow, a thymic tissue, a lymph node tissue, a spleen tissue, a tissue of the tonsil, and/or a tissue of the Peyer's patches,
further optionally wherein the method further comprises analyzing the level of CRISPR-mediated gene editing events in said one or more target cells or said one or more target tissues.

21. A method of effecting CRISPR-mediated gene editing of the HTT gene in one or more target cells and/or one or more target tissues in vivo in a subject in need thereof, the method comprising administering the composition of any one of claims 11-19 to the subject, optionally wherein:
(I) the one or more target cells comprise one or more of the following:
(i) a mammalian cell, a human cell, a cell line, a stem cell-derived cell, an induced pluripotent stem cell (iPSC)-derived cell, a primary cell, and/or a cell derived from a subject who has or has a risk of developing HD;
(ii) a brain cell, optionally a neural cell, a cortical neuron, a cell of the basal ganglia, a cell of the striatum, a cell of the caudate nucleus, and/or a cell of the putamen,
(iii) a myocyte, optionally a skeletal myocyte, a cardiomyocyte, and/or a smooth myocyte;
(iv) a cell of an endocrinal system, optionally a pancreatic cell and/or an adipocyte;
(v) a blood cell, optionally a lymphocyte, a macrophage, and/or a monocyte; and/or
(vi) a fibroblast and/or a lymphoblast; and/or
(II) the one or more target tissues comprise one or more of the following:
(vii) a mammalian tissue, a human tissue, a primary tissue, and/or a tissue from a subject who has or has a risk of developing HD;
(viii) a CNS tissue, a brain tissue, optionally a nervous tissue, a tissue of the basal ganglia, a tissue of the striatum, a tissue of the caudate nucleus, and/or a tissue of the putamen;
(ix) a muscle tissue, optionally a skeletal muscle tissue, a cardiac tissue, and/or a smooth muscle tissue;
(x) an endocrine tissue, optionally a pancreatic tissue and/or an adipose tissue; and/or
(xi) a lymphoid and/or myeloid tissue, optionally a tissue of the bone marrow, a thymic tissue, a lymph node tissue, a spleen tissue, a tissue of the tonsil, and/or a tissue of the Peyer's patches,
and optionally wherein the administrating is effected to reach a minimum of about 10%, about 15%, about 20%, about 30%, or an about final 15-30% or about final 20-40% target cells and/or tissue(s) with successful gene editing among the total target cells and/or tissue(s), further optionally wherein the method is for preventing, ameliorating, slowing the progression of, and/or treating HD and/or one or more symptoms of HD in the subject.

22. The method of claim 21, wherein the effect of the method is evaluated based on any one or more of the following:
(I) the number of the target cells with successful gene editing;
(II) the % of the target cells with successful gene editing among total target cells;
(III) the % of the target tissue(s) with successful gene editing out of total target tissue(s);
(IV) the expression level of the HTT gene in the one or more target cells;
(V) the expression level of the HTT gene in the one or more target tissues; and/or
(VI) changes in any one or more of the following:
(a) the level of aggregation of huntingtin protein, optionally in the one or more target cells, further optionally in neural cells, brain cells, and/or muscle cells,
(b) the level of aggregation of huntingtin protein in one or more target tissues, optionally in the brain,
(c) the level of cell death, optionally of the one or more target cells, further optionally of neural cells, brain cells, and/or muscle cells, optionally via apoptosis or autophagy,
(d) the level of cell death in the one or more target tissues, optionally in the brain, the basal ganglia, the striatum, the caudate nucleus, and/or the putamen, optionally via apoptosis or autophagy,
(e) the level of inflammation, optionally the level of one or more cytokines (optionally IL-4, IL-6, and/or TNF-alpha), optionally produced or released by the one or more target cells, further optionally neural cells, brain cells, immune cells, macrophages, and/or monocytes,
(f) the level of inflammation, optionally the level of one or more cytokines (optionally IL-4, IL-6, and/or TNF-alpha), in the one or more target tissues, optionally the brain, the basal ganglia, the striatum, the caudate nucleus, and/or the putamen,
(g) mitochondrial function, optionally in the one or more target cells, further optionally in lymphocytes and/or myocytes, further optionally cardiomyocytes and/or skeletal myocytes,
(h) activity or activation of caspase, optionally caspase 3 and/or caspase 9, optionally in the one or more target cells, further optionally in myocytes,
(i) the level of insulin expression and/or release by pancreatic cells, and/or
(j) the level of leptin release by adipose tissue.

23. A method of preventing, ameliorating, slowing the progression of, and/or treating HD and/or one or more symptoms of HD in a subject in need thereof, the method comprising administering the composition of any one of claims 11-19 to the subject, optionally according to the method of claim 17 or 18,
optionally wherein:
(I) the method further comprises administering one or more agents for preventing, ameliorating, slowing the progression of, or treating HD, which optionally comprise(s):
(A) a drug for controlling movements, optionally tetrabenazine, deutetrabenazine, haloperidol, fluphenazine, risperidone, olanzapine, quetiapine, amantadine, levetiracetam, and/or clonazepam; and/or
(B) a drug for controlling psychiatric disorders, optionally (a) an antidepressant, optionally citalopram, escitalopram, fluoxetine, and/or sertraline, (b) an antipsychotic drug, optionally quetiapine, risperidone, and olanzapine, and/or (c) a mood-stabilizing drug, optionally divalproex, carbamazepine, and/or lamotrigine,
optionally wherein the one or more agents is/are administered (i) separately from the composition or (ii) together with the composition; and/or
(II) the effect of the method is evaluated based on the changes in (the) one or more symptoms of HD,
and optionally wherein the one or more symptoms of HD is any one or more of the following:
(a) cognitive deficits, optionally (i) cognitive slowing, (ii) decreases in attention, and/or (iii) decreases in mental flexibility;
(b) psychiatric disruption and/or emotional deficits, optionally (i) depression, (ii) apathy, (iii) irritability, (iv) impulsivity, and/or (v) social disinhibition;
(c) loss of motor coordination, optionally (i) choreiform movements, (ii) gait disturbances, and/or (ii) bradykinesia, and/or (iv) rigidity;
(d) weight loss; and/or
(e) inflammation, optionally neural inflammation or inflammation in the brain.

24. The method of any one of claims 21-23, comprising any one or more of the following features:
(I) the subject is a mammal, optionally:
(i) a human subject, optionally a human subject who has or has a risk of developing HD; or
(ii) a non-human subject, optionally a non-human primate or selected from a rodent (mouse, rat, guinea pig, hamster), rabbit, cat, dog, pig, goat, sheep, horse, or monkey, further optionally a mouse or a rat.
(II) the composition is administered:
(A) parenterally, optionally:
(i) by injection, further optionally: direct injection into the central nervous system (CNS); intracranial injection; direct injection into the brain; direct injection into the basal ganglia; direct injection into the striatum; direct injection into the caudate nucleus and/or putamen; intrathecal (IT) injection; intraspinal (IS) injection; intravenous (IV) injection; subcutaneous (SC) injection; intramuscular (IM) injection; intradermal (ID) injection; intra-arterial (IA) injection; intraperitoneal (IP) injection; or intravitreal injection,
(ii) by inhalation or via a pulmonary route, or
(iii) transdermally;
(B) locally, optionally topically to the skin or mucous membrane; or
(C) enterally, optionally orally, sublingually/buccally, or rectally;
(III) the amount of the gRNA(s) or the gRNA-encoding polynucleotide(s) comprised in the composition per mL is about 300 to 30000 nmol, optionally about 500 to 10000 nmol, about 1000 to 5000 nmol, about 2000 to 4000 nmol, about 2500 to 3000 nmol, or about 2700 nmol;
(IV) in the administering step, the total volume comprising the composition administered is about 0.1-10000 μL, about 1-5000 μL, about 2-2000 μL, about 4-1000 μL, about 10-500 μL, or about 20-200 μL;
(V) the administering comprises injecting the composition in a continuous flow of about 0.1 μL to 2000 μL per minute, optionally about 1 μL to 1000 μL per minute, about 2 μL to 500 μL per minute, or about 5 μL to about 100 μL per minute into the brain of the subject;
(VI) the administering is effected twice or more, optionally about 3-5 times, optionally at different time points, further optionally with an interval of about a week, about two weeks, or about three weeks, about a month, about three months, about six months, about a year, about three years, or about six years;
(VII) the administering comprises at least two simultaneous or essentially simultaneous administrations at multiple sites of the subject, optionally in each hemisphere of the brain, further optionally in each striatum;
(VIII) the subject (a) is about 20 years old or older, about 25 years old or older, about 30 years old or older, about 35 years old or older, about 40 years old or older or (b) is juvenile; and/or
(IX) the subject (a) has not fully developed HD and/or is has not manifested a symptom or complication or (b) is at initial onset of HD.

25. A kit comprising:
(i) the composition of any one of claims 11-19, optionally in a vessel; and
(ii) an instruction and/or a label,
optionally wherein the instruction is for use of the composition, optionally according to the method of any one of claims 20-24.

26. The composition of any one of claims 11-19, for use as a medicament, optionally for preventing, ameliorating, slowing the progression of, and/or treating HD and/or for preventing, ameliorating, slowing the progression of, and/or treating one or more symptoms of HD, further optionally according to the method of any one of claims 20-24.

27. Use of the composition of any one of claims 11-19 for the manufacture of a medicament for preventing, ameliorating, slowing the progression of, and/or treating HD or for preventing, ameliorating, slowing the progression of, and/or treating one or more symptoms of HD, optionally according to the method of any one of claims 20-24.


SEQUENCE LISTING (611 sequences)

SEQ ID NO: 1 - DNA, 20 bp, Artificial Sequence
  Ex31-1 gRNA targeting sequence (20nt) as DNA
agataagccatcaaactggg

SEQ ID NO: 2 - DNA, 20 bp, Artificial Sequence
  Ex31-2 gRNA targeting sequence (20nt) as DNA
gtacaagcctggcctcacac

SEQ ID NO: 3 - DNA, 20 bp, Artificial Sequence
  Ex31-3 gRNA targeting sequence (20nt) as DNA
ccgcctgcaccatgttcctc

SEQ ID NO: 4 - DNA, 20 bp, Artificial Sequence
  Ex5-1 gRNA targeting sequence (20nt) as DNA
cccctcagaatggtgcccct

SEQ ID NO: 5 - DNA, 20 bp, Artificial Sequence
  Ex5-2 gRNA targeting sequence (20nt) as DNA
gtttgctgagctggctcacc

SEQ ID NO: 6 - DNA, 20 bp, Artificial Sequence
  Ex5-3 gRNA targeting sequence (20nt) as DNA
acaacttacctgcatttctg

SEQ ID NO: 7 - DNA, 20 bp, Artificial Sequence
  Ex8-1 gRNA targeting sequence (20nt) as DNA
aggcttactcgttcctgtcg

SEQ ID NO: 8 - DNA, 20 bp, Artificial Sequence
  Ex8-2 gRNA targeting sequence (20nt) as DNA
gctgctcaccctgaggtatt

SEQ ID NO: 9 - DNA, 20 bp, Artificial Sequence
  Ex8-3 gRNA targeting sequence (20nt) as DNA
gtcaaggacacaagcctgaa

SEQ ID NO: 10 - RNA, 20 nt, Artificial Sequence
  Ex31-1 gRNA targeting sequence (20nt) as RNA
agauaagccaucaaacuggg

SEQ ID NO: 11 - RNA, 20 nt, Artificial Sequence
  Ex31-2 gRNA targeting sequence (20nt) as RNA
guacaagccuggccucacac

SEQ ID NO: 12 - RNA, 20 nt, Artificial Sequence
  Ex31-3 gRNA targeting sequence (20nt) as RNA
ccgccugcaccauguuccuc

SEQ ID NO: 13 - RNA, 20 nt, Artificial Sequence
  Ex5-1 gRNA targeting sequence (20nt) as RNA
ccccucagaauggugccccu

SEQ ID NO: 14 - RNA, 20 nt, Artificial Sequence
  Ex5-2 gRNA targeting sequence (20nt) as RNA
guuugcugagcuggcucacc

SEQ ID NO: 15 - RNA, 20 nt, Artificial Sequence
  Ex5-3 gRNA targeting sequence (20nt) as RNA
acaacuuaccugcauuucug

SEQ ID NO: 16 - RNA, 20 nt, Artificial Sequence
  Ex8-1 gRNA targeting sequence (20nt) as RNA
aggcuuacucguuccugucg

SEQ ID NO: 17 - RNA, 19 nt, Artificial Sequence
  Ex8-2 gRNA targeting sequence (20nt) as RNA
gcugcucacccugagguau

SEQ ID NO: 18 - RNA, 20 nt, Artificial Sequence
  Ex8-3 gRNA targeting sequence (20nt) as RNA
gucaaggacacaagccugaa

SEQ ID NO: 19 - RNA, 20 nt, Artificial Sequence
  m-Ex31-1 gRNA targeting sequence (20nt) as RNA
agauaagccaucaaacugug

SEQ ID NO: 20 - RNA, 20 nt, Artificial Sequence
  m-Ex31-2 gRNA targeting sequence (20nt) as RNA
gccucacacuugaagagcca

SEQ ID NO: 21 - RNA, 20 nt, Artificial Sequence
  m-Ex31-3 gRNA targeting sequence (20nt) as RNA
ccgccugcaccauguuccuc

SEQ ID NO: 22 - RNA, 20 nt, Artificial Sequence
  Lucuferase gRNA targeting sequence (20nt) as RNA
cuucgaaauguccguugggu

SEQ ID NO: 23 - RNA, 20 nt, Artificial Sequence
  Ex2-1 gRNA targeting sequence (20nt) as RNA
augauucacacggucuuucu

SEQ ID NO: 24-29 - skipped (6 entries)

SEQ ID NO: 30 - DNA, 20 bp, Artificial Sequence
  Ex2-1 gRNA targeting sequence (20nt) as DNA
atgattcacacggtctttct

SEQ ID NO: 31-100 - skipped (70 entries)

SEQ ID NO: 101 - RNA, 12 nt, Artificial Sequence
  crRNA flagpole (option 1)
guuuuagagcua

SEQ ID NO: 102 - RNA, 12 nt, Artificial Sequence
  crRNA flagpole (option 2)
guuuaagagcua

SEQ ID NO: 103 - RNA, 5 nt, Artificial Sequence
  crRNA first flagpole extension (optional)
ugcug

SEQ ID NO: 104 - RNA, 5 nt, Artificial Sequence
  crRNA second flagpole extension (optional)
uuuug

SEQ ID NO: 105 - RNA, 5 nt, Artificial Sequence
  tracrRNA first extension (optional)
cagca

SEQ ID NO: 106 - RNA, 13 nt, Artificial Sequence
  tracrRNA flagpole (option 1)
uagcaaguuaaaa

SEQ ID NO: 107 - RNA, 13 nt, Artificial Sequence
  tracrRNA flagpole (option 2)
uagcaaguuuaaa

SEQ ID NO: 108 - RNA, 47 nt, Artificial Sequence
  tracrRNA nuclease binding domain (may be followed by multiple uracils)
uaaggcuaguccguuaucaacuugaaaaaguggcaccgagucggugc

SEQ ID NO: 109 - RNA, 4 nt, Artificial Sequence
  linker (for linking crRNA sequence and tracrRNA sequence)
gaaa

SEQ ID NO: 110 - skipped

SEQ ID NO: 111 - RNA, 76 nt, Artificial Sequence
  sgRNA backbone (option 1; placed 3′ to targeting sequence; may be followed by multiple uracils)
guuuuagagcuagaaauagcaaguuaaaauaaggcuaguccguuaucaacuugaaaaaguggcaccgagucggugc

SEQ ID NO: 112 - RNA, 76 nt, Artificial Sequence
  sgRNA backbone (option 2; placed 3′ to targeting sequence; may be followed by multiple uracils)
guuuaagagcuagaaauagcaaguuuaaauaaggcuaguccguuaucaacuugaaaaaguggcaccgagucggugc

SEQ ID NO: 113 - RNA, 86 nt, Artificial Sequence
  sgRNA backbone (option 3; placed 3′ to targeting sequence; may be followed by multiple uracils)
guuuuagagcuaugcuggaaacagcauagcaaguuaaaauaaggcuaguccguuaucaacuugaaaaaguggcaccgagucggugc

SEQ ID NO: 114 - RNA, 86 nt, Artificial Sequence
  sgRNA backbone (option 4; placed 3′ to targeting sequence; may be followed by multiple uracils)
guuuaagagcuaugcuggaaacagcauagcaaguuuaaauaaggcuaguccguuaucaacuugaaaaaguggcaccgagucggugc

SEQ ID NO: 115 - RNA, 22 nt, Artificial Sequence
  dgRNA crRNA backbone (option 1)
guuuuagagcuaugcuguuuug

SEQ ID NO: 116 - RNA, 74 nt, Artificial Sequence
  dgRNA tracrRNA (option 1)
aacagcauagcaaguuaaaauaaggcuaguccguuaucaacuugaaaaaguggcaccgagucggugcuuuuuuu

SEQ ID NO: 117 - RNA, 22 nt, Artificial Sequence
  dgRNA crRNA backbone (option 2)
guuuaagagcuaugcuguuuug

SEQ ID NO: 118 - RNA, 74 nt, Artificial Sequence
  dgRNA tracrRNA (option 2)
aacagcauagcaaguuuaaauaaggcuaguccguuaucaacuugaaaaaguggcaccgagucggugcuuuuuuu

SEQ ID NO: 119-199 - skipped (81 entries)

SEQ ID NO: 200 - DNA, 80 bp, Artificial Sequence
  Human HTT Exon 5
aatggtgcccctcggagtttgcgtgctgccctgtggaggtttgctgagctggctcacctggttcggcctcagaaatgcag

SEQ ID NO: 201 - DNA, 1494 bp, Artificial Sequence
  Human HTT Exon 5-containing segment
gggagattgaggtgggaggattaattgagcctggaaggttgaatctataggtagctgagattgtgccactgcccttcagcctgggcgaccaagtgagaccctgtctcaaaagaaaaacaaaaaaacaaaaaacaaaccactattatcgactatatattattgtctatgatccctctgctgtgctgtcgaataccaggtcttgggcccttatttccatcactgagcaaacttcactctgttaagcagcaggtgtgggatttcatcgttattcagtaattcacaatgttagaaggaaatgctgtttggtagacgattgctttacttttcttcaaaaggttactctttattagatgagatgagaattaaaaatggtaacttactttatatctttataattgaagcccactagaccttaaagtagttaccagatgttttatgcatttaaatggccttttctctaaaattagaaagtaacaaggaaagaaaatgcttcgtttctatgcaaccctcttggtgactagtatgtgactcttaatgcaaccctcattgcaccccctcagaatggtgcccctcggagtttgcgtgctgccctgtggaggtttgctgagctggctcacctggttcggcctcagaaatgcaggtaagttgtacactctggatgttggtttttgtcgggggccagctgctactgatcctttatgtctcagctcagatgtcatttcaaaagtctgctctgccctctccaaattgcagtcgaccttgccctgtttatgtttccctcatagcactaatccatgtcagaaattgtcacgtacagtctatctgtgtgcttgtttattttctatcccacccttccgcaagagacttatgggatgtgtgccccaggacagcaggggtcttactgtcttatgctctgttgcagcccagcagcgataacagtgtctgcacatagtacttgcttaaaagatacttgccaaattgttgaaggttgaggtaccaatttcattattgctgactataggagttatagcaaaatatccatttgtctgttacatgagttaaaaatatggttgttgcactgtgaatagtttggtttagtcaaaacagttgtatcttaacggattgagaaacaaaagcaggaccacttttcatcagctccctccttctccttaaccagcaatacatgctgatgctgatatcccatagaccctcagctccatcctgagtcactgggaatgtggtctaaaccctcactattaatatgaactgagtttcaataagaatcttatatgggtcgggcatagtggctcatacctttgatcccagcacttcaggaggccaaggcaggtggattgcttgacccagactaggcaacatggtgaaacgccgcctctacaaaaaatacaaaacttagccaggcatggtggtgcgtgcctgtggtcacagccactcgagaggctgaggtgggaggatcacttgagcctgggaggtgga

SEQ ID NO: 202 - DNA, 140 bp, Artificial Sequence
  Human HTT Exon 5 plus 30 nt each end
cttaatgcaaccctcattgcaccccctcagaatggtgcccctcggagtttgcgtgctgccctgtggaggtttgctgagctggctcacctggttcggcctcagaaatgcaggtaagttgtacactctggatgttggttttt

SEQ ID NO: 203-204 - skipped (2 entries)

SEQ ID NO: 205 - DNA, 80 bp, Artificial Sequence
  Reverse complementary sequence of SEQ ID NO: 200
ctgcatttctgaggccgaaccaggtgagccagctcagcaaacctccacagggcagcacgcaaactccgaggggcaccatt

SEQ ID NO: 206 - DNA, 1494 bp, Artificial Sequence
  Reverse complementary sequence of SEQ ID NO: 201
tccacctcccaggctcaagtgatcctcccacctcagcctctcgagtggctgtgaccacaggcacgcaccaccatgcctggctaagttttgtattttttgtagaggcggcgtttcaccatgttgcctagtctgggtcaagcaatccacctgccttggcctcctgaagtgctgggatcaaaggtatgagccactatgcccgacccatataagattcttattgaaactcagttcatattaatagtgagggtttagaccacattcccagtgactcaggatggagctgagggtctatgggatatcagcatcagcatgtattgctggttaaggagaaggagggagctgatgaaaagtggtcctgcttttgtttctcaatccgttaagatacaactgttttgactaaaccaaactattcacagtgcaacaaccatatttttaactcatgtaacagacaaatggatattttgctataactcctatagtcagcaataatgaaattggtacctcaaccttcaacaatttggcaagtatcttttaagcaagtactatgtgcagacactgttatcgctgctgggctgcaacagagcataagacagtaagacccctgctgtcctggggcacacatcccataagtctcttgcggaagggtgggatagaaaataaacaagcacacagatagactgtacgtgacaatttctgacatggattagtgctatgagggaaacataaacagggcaaggtcgactgcaatttggagagggcagagcagacttttgaaatgacatctgagctgagacataaaggatcagtagcagctggcccccgacaaaaaccaacatccagagtgtacaacttacctgcatttctgaggccgaaccaggtgagccagctcagcaaacctccacagggcagcacgcaaactccgaggggcaccattctgagggggtgcaatgagggttgcattaagagtcacatactagtcaccaagagggttgcatagaaacgaagcattttctttccttgttactttctaattttagagaaaaggccatttaaatgcataaaacatctggtaactactttaaggtctagtgggcttcaattataaagatataaagtaagttaccatttttaattctcatctcatctaataaagagtaaccttttgaagaaaagtaaagcaatcgtctaccaaacagcatttccttctaacattgtgaattactgaataacgatgaaatcccacacctgctgcttaacagagtgaagtttgctcagtgatggaaataagggcccaagacctggtattcgacagcacagcagagggatcatagacaataatatatagtcgataatagtggtttgttttttgtttttttgtttttcttttgagacagggtctcacttggtcgcccaggctgaagggcagtggcacaatctcagctacctatagattcaaccttccaggctcaattaatcctcccacctcaatctccc

SEQ ID NO: 207 - DNA, 140 bp, Artificial Sequence
  Reverse complementary sequence of SEQ ID NO: 202
aaaaaccaacatccagagtgtacaacttacctgcatttctgaggccgaaccaggtgagccagctcagcaaacctccacagggcagcacgcaaactccgaggggcaccattctgagggggtgcaatgagggttgcattaag

SEQ ID NO: 208-209 - skipped (2 entries)

SEQ ID NO: 210 - DNA, 20 bp, Artificial Sequence
  Ex5-1 gRNA targeting sequence (20nt)
cccctcagaatggtgcccct

SEQ ID NO: 211 - DNA, 19 bp, Artificial Sequence
  Ex5-1 gRNA targeting sequence (19nt)
ccctcagaatggtgcccct

SEQ ID NO: 212 - DNA, 18 bp, Artificial Sequence
  Ex5-1 gRNA targeting sequence (18nt)
cctcagaatggtgcccct

SEQ ID NO: 213 - DNA, 17 bp, Artificial Sequence
  Ex5-1 gRNA targeting sequence (17nt)
ctcagaatggtgcccct

SEQ ID NO: 214-219 - skipped (6 entries)

SEQ ID NO: 220 - DNA, 20 bp, Artificial Sequence
  Ex5-2 gRNA targeting sequence (20nt)
gtttgctgagctggctcacc

SEQ ID NO: 221 - DNA, 19 bp, Artificial Sequence
  Ex5-2 gRNA targeting sequence (19nt)
tttgctgagctggctcacc

SEQ ID NO: 222 - DNA, 18 bp, Artificial Sequence
  Ex5-2 gRNA targeting sequence (18nt)
ttgctgagctggctcacc

SEQ ID NO: 223 - DNA, 17 bp, Artificial Sequence
  Ex5-2 gRNA targeting sequence (17nt)
tgctgagctggctcacc

SEQ ID NO: 224-229 - skipped (6 entries)

SEQ ID NO: 230 - DNA, 20 bp, Artificial Sequence
  Ex5-3 gRNA targeting sequence (20nt)
acaacttacctgcatttctg

SEQ ID NO: 231 - DNA, 19 bp, Artificial Sequence
  Ex5-3 gRNA targeting sequence (19nt)
caacttacctgcatttctg

SEQ ID NO: 232 - DNA, 18 bp, Artificial Sequence
  Ex5-3 gRNA targeting sequence (18nt)
aacttacctgcatttctg

SEQ ID NO: 233 - DNA, 17 bp, Artificial Sequence
  Ex5-3 gRNA targeting sequence (17nt)
acttacctgcatttctg

SEQ ID NO: 234-260 - skipped (27 entries)

SEQ ID NO: 261 - DNA, 10 bp, Artificial Sequence
  Ex5 ssODN 5′ homology arm (10 nt)
agaatggtgc

SEQ ID NO: 262 - DNA, 20 bp, Artificial Sequence
  Ex5 ssODN 5′ homology arm (20 nt)
gcaccccctcagaatggtgc

SEQ ID NO: 263 - DNA, 30 bp, Artificial Sequence
  Ex5 ssODN 5′ homology arm (30 nt)
aaccctcattgcaccccctcagaatggtgc

SEQ ID NO: 264 - DNA, 40 bp, Artificial Sequence
  Ex5 ssODN 5′ homology arm (40 nt)
ctcttaatgcaaccctcattgcaccccctcagaatggtgc

SEQ ID NO: 265 - DNA, 50 bp, Artificial Sequence
  Ex5 ssODN 5′ homology arm (50 nt)
tagtatgtgactcttaatgcaaccctcattgcaccccctcagaatggtgc

SEQ ID NO: 266 - DNA, 60 bp, Artificial Sequence
  Ex5 ssODN 5′ homology arm (60 nt)
tcttggtgactagtatgtgactcttaatgcaaccctcattgcaccccctcagaatggtgc

SEQ ID NO: 267 - DNA, 70 bp, Artificial Sequence
  Ex5 ssODN 5′ homology arm (70 nt)
tatgcaaccctcttggtgactagtatgtgactcttaatgcaaccctcattgcaccccctcagaatggtgc

SEQ ID NO: 268 - DNA, 80 bp, Artificial Sequence
  Ex5 ssODN 5′ homology arm (80 nt)
gcttcgtttctatgcaaccctcttggtgactagtatgtgactcttaatgcaaccctcattgcaccccctcagaatggtgc

SEQ ID NO: 269-270 - skipped (2 entries)

SEQ ID NO: 271 - DNA, 10 bp, Artificial Sequence
  Ex5 ssODN 3′ homology arm (10 nt)
aatgcaggta

SEQ ID NO: 272 - DNA, 20 bp, Artificial Sequence
  Ex5 ssODN 3′ homology arm (20 nt)
aatgcaggtaagttgtacac

SEQ ID NO: 273 - DNA, 30 bp, Artificial Sequence
  Ex5 ssODN 3′ homology arm (30 nt)
aatgcaggtaagttgtacactctggatgtt

SEQ ID NO: 274 - DNA, 40 bp, Artificial Sequence
  Ex5 ssODN 3′ homology arm (40 nt)
aatgcaggtaagttgtacactctggatgttggtttttgtc

SEQ ID NO: 275 - DNA, 50 bp, Artificial Sequence
  Ex5 ssODN 3′ homology arm (50 nt)
aatgcaggtaagttgtacactctggatgttggtttttgtcgggggccagc

SEQ ID NO: 276 - DNA, 60 bp, Artificial Sequence
  Ex5 ssODN 3′ homology arm (60 nt)
aatgcaggtaagttgtacactctggatgttggtttttgtcgggggccagctgctactgat

SEQ ID NO: 277 - DNA, 70 bp, Artificial Sequence
  Ex5 ssODN 3′ homology arm (70 nt)
aatgcaggtaagttgtacactctggatgttggtttttgtcgggggccagctgctactgatcctttatgtc

SEQ ID NO: 278 - DNA, 80 bp, Artificial Sequence
  Ex5 ssODN 3′ homology arm (80 nt)
aatgcaggtaagttgtacactctggatgttggtttttgtcgggggccagctgctactgatcctttatgtctcagctcaga

SEQ ID NO: 279-280 - skipped (2 entries)

SEQ ID NO: 281 - DNA, 20 bp, Artificial Sequence
  Ex5 ssODN sequence 1 (10 nt each arm)
agaatggtgcaatgcaggta

SEQ ID NO: 282 - DNA, 40 bp, Artificial Sequence
  Ex5 ssODN sequence 2 (20 nt each arm)
gcaccccctcagaatggtgcaatgcaggtaagttgtacac

SEQ ID NO: 283 - DNA, 60 bp, Artificial Sequence
  Ex5 ssODN sequence 3 (30 nt each arm)
aaccctcattgcaccccctcagaatggtgcaatgcaggtaagttgtacactctggatgtt

SEQ ID NO: 284 - DNA, 80 bp, Artificial Sequence
  Ex5 ssODN sequence 4 (40 nt each arm)
ctcttaatgcaaccctcattgcaccccctcagaatggtgcaatgcaggtaagttgtacactctggatgttggtttttgtc

SEQ ID NO: 285 - DNA, 100 bp, Artificial Sequence
  Ex5 ssODN sequence 5 (50 nt each arm)
tagtatgtgactcttaatgcaaccctcattgcaccccctcagaatggtgcaatgcaggtaagttgtacactctggatgttggtttttgtcgggggccagc

SEQ ID NO: 286 - DNA, 120 bp, Artificial Sequence
  Ex5 ssODN sequence 6 (60 nt each arm)
tcttggtgactagtatgtgactcttaatgcaaccctcattgcaccccctcagaatggtgcaatgcaggtaagttgtacactctggatgttggtttttgtcgggggccagctgctactgat

SEQ ID NO: 287 - DNA, 140 bp, Artificial Sequence
  Ex5 ssODN sequence 7 (70 nt each arm)
tatgcaaccctcttggtgactagtatgtgactcttaatgcaaccctcattgcaccccctcagaatggtgcaatgcaggtaagttgtacactctggatgttggtttttgtcgggggccagctgctactgatcctttatgtc

SEQ ID NO: 288 - DNA, 160 bp, Artificial Sequence
  Ex5 ssODN sequence 8 (80 nt each arm)
gcttcgtttctatgcaaccctcttggtgactagtatgtgactcttaatgcaaccctcattgcaccccctcagaatggtgcaatgcaggtaagttgtacactctggatgttggtttttgtcgggggccagctgctactgatcctttatgtctcagctcaga

SEQ ID NO: 289-290 - skipped (2 entries)

SEQ ID NO: 291 - DNA, 20 bp, Artificial Sequence
  Ex5 ssODN sequence 1a (10 nt each arm)
tacctgcattgcaccattct

SEQ ID NO: 292 - DNA, 40 bp, Artificial Sequence
  Ex5 ssODN sequence 2a (20 nt each arm)
gtgtacaacttacctgcattgcaccattctgagggggtgc

SEQ ID NO: 293 - DNA, 60 bp, Artificial Sequence
  Ex5 ssODN sequence 3a (30 nt each arm)
aacatccagagtgtacaacttacctgcattgcaccattctgagggggtgcaatgagggtt

SEQ ID NO: 294 - DNA, 80 bp, Artificial Sequence
  Ex5 ssODN sequence 4a (40 nt each arm)
gacaaaaaccaacatccagagtgtacaacttacctgcattgcaccattctgagggggtgcaatgagggttgcattaagag

SEQ ID NO: 295 - DNA, 100 bp, Artificial Sequence
  Ex5 ssODN sequence 5a (50 nt each arm)
gctggcccccgacaaaaaccaacatccagagtgtacaacttacctgcattgcaccattctgagggggtgcaatgagggttgcattaagagtcacatacta

SEQ ID NO: 296 - DNA, 120 bp, Artificial Sequence
  Ex5 ssODN sequence 6a (60 nt each arm)
atcagtagcagctggcccccgacaaaaaccaacatccagagtgtacaacttacctgcattgcaccattctgagggggtgcaatgagggttgcattaagagtcacatactagtcaccaaga

SEQ ID NO: 297 - DNA, 140 bp, Artificial Sequence
  Ex5 ssODN sequence 7a (70 nt each arm)
gacataaaggatcagtagcagctggcccccgacaaaaaccaacatccagagtgtacaacttacctgcattgcaccattctgagggggtgcaatgagggttgcattaagagtcacatactagtcaccaagagggttgcata

SEQ ID NO: 298 - DNA, 160 bp, Artificial Sequence
  Ex5 ssODN sequence 8a (80 nt each arm)
tctgagctgagacataaaggatcagtagcagctggcccccgacaaaaaccaacatccagagtgtacaacttacctgcattgcaccattctgagggggtgcaatgagggttgcattaagagtcacatactagtcaccaagagggttgcatagaaacgaagc

SEQ ID NO: 299 - skipped

SEQ ID NO: 300 - DNA, 179 bp, Artificial Sequence
  Human HTT Exon 8
gcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccag

SEQ ID NO: 301 - DNA, 1491 bp, Artificial Sequence
  Human HTT Exon 8-containing segment
taaggtggaggcatatgagtggaagagtctccagcatgtactcaagatagacctttgaaataaataaaaccagatgatccctcagcttctagaccaggctatttggcactggttgattgaatgtgaactgcactggggctgctgtgagcccgcatgggtctctgtgaccctgcagatgcagccgtgcccagggactgggcagtgggtgtgggctggtgtgagccctgtctgccacccagggcctggccctctgtctgtgtcggccatgactatggtgagtcttgtaggcttgagactgtgcctcgggttcctgcgggttctctgtaggtcagttgacagtttctcctgttgtttgggtaactgtggaaacgaacactggcaagtgctgaagcgagcatgtggacgtgcgatatgaaataacgacctggctttcaaaggcagtgaggctctctggaaaggaccttgctgagctagggatgtgggtgtgtagccattcccagtgggcctcatggcgtactcgttcatgatcatgtttgtgccatcttgatctctcaggatctcttcttttttaacagattaagccgggaatctccaaacagtgagtcagatgttaagatgtcttgcttccacccccacaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtaggagcacagggtttactctaggccctgcatgtgaatgactgacattcaaagaaccgattaatttggaagagaagcggcagaaccgagagttagaggtgtggactctggagctgcgctgctcgtttccaaccctaggtgctgacctctagctgtcttccctctgtatgtccctgtcaccgtgagtcaaatgcgggtgatgcctcctcaggtgccgtgttacctaagcctctcagagaccactgctaccctgtttctaaaaccagaggtcacgatatgtgttcatccacccagtaaatactgattgagcacccactgtgtgctaggctctgggataggggctgggtatacaatggtgagtatttcagctgcagcttctgccccgtggaggctgtggcctagcacactggtctaggcacggtggtatatgctcactcaaggagatagggacgtggtcgtttggggtgtcggaacaaaatgtcggaacttctctttccaatgcagagaaaccttgcagtaattctaatgtactgtgattggcagttgacttcagttctttgtagcacgcttactcaggttatttcactaactatgtaaccatgcagcctcattttaagcaattggattttttgaactttacttaaaatgttatgtcagggtttttattgtgctta

SEQ ID NO: 302 - DNA, 242 bp, Artificial Sequence
  Human HTT Exon 8 plus 30 nt each end
agatgttaagatgtcttgcttccacccccacaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtaggagcacagggtttactctaggccctg

SEQ ID NO: 303-304 - skipped (2 entries)

SEQ ID NO: 305 - DNA, 179 bp, Artificial Sequence
  Reverse complementary sequence of SEQ ID NO: 300
ctggacaagctgctctgcagaaggagagacttccatttctttccttgtcactccgaagctgcctttcaggcttgtgtccttgacctgctgctgcagcaagggcaccaaatacctcagggtgagcagcacgccaagaatcagcagagtggagtgttcatcctcgacaggaacgagtaagc

SEQ ID NO: 306 - DNA, 1491 bp, Artificial Sequence
  Reverse complementary sequence of SEQ ID NO: 301
taagcacaataaaaaccctgacataacattttaagtaaagttcaaaaaatccaattgcttaaaatgaggctgcatggttacatagttagtgaaataacctgagtaagcgtgctacaaagaactgaagtcaactgccaatcacagtacattagaattactgcaaggtttctctgcattggaaagagaagttccgacattttgttccgacaccccaaacgaccacgtccctatctccttgagtgagcatataccaccgtgcctagaccagtgtgctaggccacagcctccacggggcagaagctgcagctgaaatactcaccattgtatacccagcccctatcccagagcctagcacacagtgggtgctcaatcagtatttactgggtggatgaacacatatcgtgacctctggttttagaaacagggtagcagtggtctctgagaggcttaggtaacacggcacctgaggaggcatcacccgcatttgactcacggtgacagggacatacagagggaagacagctagaggtcagcacctagggttggaaacgagcagcgcagctccagagtccacacctctaactctcggttctgccgcttctcttccaaattaatcggttctttgaatgtcagtcattcacatgcagggcctagagtaaaccctgtgctcctacctggacaagctgctctgcagaaggagagacttccatttctttccttgtcactccgaagctgcctttcaggcttgtgtccttgacctgctgctgcagcaagggcaccaaatacctcagggtgagcagcacgccaagaatcagcagagtggagtgttcatcctcgacaggaacgagtaagcctgtgggggtggaagcaagacatcttaacatctgactcactgtttggagattcccggcttaatctgttaaaaaagaagagatcctgagagatcaagatggcacaaacatgatcatgaacgagtacgccatgaggcccactgggaatggctacacacccacatccctagctcagcaaggtcctttccagagagcctcactgcctttgaaagccaggtcgttatttcatatcgcacgtccacatgctcgcttcagcacttgccagtgttcgtttccacagttacccaaacaacaggagaaactgtcaactgacctacagagaacccgcaggaacccgaggcacagtctcaagcctacaagactcaccatagtcatggccgacacagacagagggccaggccctgggtggcagacagggctcacaccagcccacacccactgcccagtccctgggcacggctgcatctgcagggtcacagagacccatgcgggctcacagcagccccagtgcagttcacattcaatcaaccagtgccaaatagcctggtctagaagctgagggatcatctggttttatttatttcaaaggtctatcttgagtacatgctggagactcttccactcatatgcctccacctta

SEQ ID NO: 307 - DNA, 242 bp, Artificial Sequence
  Reverse complementary sequence of SEQ ID NO: 302
cagggcctagagtaaaccctgtgctcctacctggacaagctgctctgcagaaggagagacttccatttctttccttgtcactccgaagctgcctttcaggcttgtgtccttgacctgctgctgcagcaagggcaccaaatacctcagggtgagcagcacgccaagaatcagcagagtggagtgttcatcctcgacaggaacgagtaagcctgtgggggtggaagcaagacatcttaacatct

SEQ ID NO: 308-309 - skipped (2 entries)

SEQ ID NO: 310 - DNA, 20 bp, Artificial Sequence
  Ex8-1 gRNA targeting sequence (20nt)
aggcttactcgttcctgtcg

SEQ ID NO: 311 - DNA, 19 bp, Artificial Sequence
  Ex8-1 gRNA targeting sequence (19nt)
ggcttactcgttcctgtcg

SEQ ID NO: 312 - DNA, 18 bp, Artificial Sequence
  Ex8-1 gRNA targeting sequence (18nt)
gcttactcgttcctgtcg

SEQ ID NO: 313 - DNA, 17 bp, Artificial Sequence
  Ex8-1 gRNA targeting sequence (17nt)
cttactcgttcctgtcg

SEQ ID NO: 314-319 - skipped (6 entries)

SEQ ID NO: 320 - DNA, 20 bp, Artificial Sequence
  Ex8-2 gRNA targeting sequence (20nt)
gctgctcaccctgaggtatt

SEQ ID NO: 321 - DNA, 19 bp, Artificial Sequence
  Ex8-2 gRNA targeting sequence (19nt)
ctgctcaccctgaggtatt

SEQ ID NO: 322 - DNA, 18 bp, Artificial Sequence
  Ex8-2 gRNA targeting sequence (18nt)
tgctcaccctgaggtatt

SEQ ID NO: 323 - DNA, 17 bp, Artificial Sequence
  Ex8-2 gRNA targeting sequence (17nt)
gctcaccctgaggtatt

SEQ ID NO: 324-329 - skipped (6 entries)

SEQ ID NO: 330 - DNA, 20 bp, Artificial Sequence
  Ex8-3 gRNA targeting sequence (20nt)
gtcaaggacacaagcctgaa

SEQ ID NO: 331 - DNA, 19 bp, Artificial Sequence
  Ex8-3 gRNA targeting sequence (19nt)
tcaaggacacaagcctgaa

SEQ ID NO: 332 - DNA, 18 bp, Artificial Sequence
  Ex8-3 gRNA targeting sequence (18nt)
caaggacacaagcctgaa

SEQ ID NO: 333 - DNA, 17 bp, Artificial Sequence
  Ex8-3 gRNA targeting sequence (17nt)
aaggacacaagcctgaa

SEQ ID NO: 334-360 - skipped (27 entries)

SEQ ID NO: 361 - DNA, 10 bp, Artificial Sequence
  Ex8 ssODN 5′ homology arm (10 nt)
actcgttcct

SEQ ID NO: 362 - DNA, 20 bp, Artificial Sequence
  Ex8 ssODN 5′ homology arm (20 nt)
ccacaggcttactcgttcct

SEQ ID NO: 363 - DNA, 30 bp, Artificial Sequence
  Ex8 ssODN 5′ homology arm (30 nt)
gcttccacccccacaggcttactcgttcct

SEQ ID NO: 364 - DNA, 40 bp, Artificial Sequence
  Ex8 ssODN 5′ homology arm (40 nt)
aagatgtcttgcttccacccccacaggcttactcgttcct

SEQ ID NO: 365 - DNA, 50 bp, Artificial Sequence
  Ex8 ssODN 5′ homology arm (50 nt)
gtcagatgttaagatgtcttgcttccacccccacaggcttactcgttcct

SEQ ID NO: 366 - DNA, 60 bp, Artificial Sequence
  Ex8 ssODN 5′ homology arm (60 nt)
caaacagtgagtcagatgttaagatgtcttgcttccacccccacaggcttactcgttcct

SEQ ID NO: 367 - DNA, 70 bp, Artificial Sequence
  Ex8 ssODN 5′ homology arm (70 nt)
cgggaatctccaaacagtgagtcagatgttaagatgtcttgcttccacccccacaggcttactcgttcct

SEQ ID NO: 368 - DNA, 80 bp, Artificial Sequence
  Ex8 ssODN 5′ homology arm (80 nt)
cagattaagccgggaatctccaaacagtgagtcagatgttaagatgtcttgcttccacccccacaggcttactcgttcct

SEQ ID NO: 369-370 - skipped (2 entries)

SEQ ID NO: 371 - DNA, 10 bp, Artificial Sequence
  Ex8 ssODN 3′ homology arm (10 nt)
tgaaaggcag

SEQ ID NO: 372 - DNA, 20 bp, Artificial Sequence
  Ex8 ssODN 3′ homology arm (20 nt)
tgaaaggcagcttcggagtg

SEQ ID NO: 373 - DNA, 30 bp, Artificial Sequence
  Ex8 ssODN 3′ homology arm (30 nt)
tgaaaggcagcttcggagtgacaaggaaag

SEQ ID NO: 374 - DNA, 40 bp, Artificial Sequence
  Ex8 ssODN 3′ homology arm (40 nt)
tgaaaggcagcttcggagtgacaaggaaagaaatggaagt

SEQ ID NO: 375 - DNA, 50 bp, Artificial Sequence
  Ex8 ssODN 3′ homology arm (50 nt)
tgaaaggcagcttcggagtgacaaggaaagaaatggaagtctctccttct

SEQ ID NO: 376 - DNA, 60 bp, Artificial Sequence
  Ex8 ssODN 3′ homology arm (60 nt)
tgaaaggcagcttcggagtgacaaggaaagaaatggaagtctctccttctgcagagcagc

SEQ ID NO: 377 - DNA, 70 bp, Artificial Sequence
  Ex8 ssODN 3′ homology arm (70 nt)
tgaaaggcagcttcggagtgacaaggaaagaaatggaagtctctccttctgcagagcagcttgtccaggt

SEQ ID NO: 378 - DNA, 80 bp, Artificial Sequence
  Ex8 ssODN 3′ homology arm (80 nt)
tgaaaggcagcttcggagtgacaaggaaagaaatggaagtctctccttctgcagagcagcttgtccaggtaggagcacag

SEQ ID NO: 379-380 - skipped (2 entries)

SEQ ID NO: 381 - DNA, 20 bp, Artificial Sequence
  Ex8 ssODN sequence 1 (10 nt each arm)
actcgttccttgaaaggcag

SEQ ID NO: 382 - DNA, 40 bp, Artificial Sequence
  Ex8 ssODN sequence 2 (20 nt each arm)
ccacaggcttactcgttccttgaaaggcagcttcggagtg

SEQ ID NO: 383 - DNA, 60 bp, Artificial Sequence
  Ex8 ssODN sequence 3 (30 nt each arm)
gcttccacccccacaggcttactcgttccttgaaaggcagcttcggagtgacaaggaaag

SEQ ID NO: 384 - DNA, 80 bp, Artificial Sequence
  Ex8 ssODN sequence 4 (40 nt each arm)
aagatgtcttgcttccacccccacaggcttactcgttccttgaaaggcagcttcggagtgacaaggaaagaaatggaagt

SEQ ID NO: 385 - DNA, 100 bp, Artificial Sequence
  Ex8 ssODN sequence 5 (50 nt each arm)
gtcagatgttaagatgtcttgcttccacccccacaggcttactcgttccttgaaaggcagcttcggagtgacaaggaaagaaatggaagtctctccttct

SEQ ID NO: 386 - DNA, 120 bp, Artificial Sequence
  Ex8 ssODN sequence 6 (60 nt each arm)
caaacagtgagtcagatgttaagatgtcttgcttccacccccacaggcttactcgttccttgaaaggcagcttcggagtgacaaggaaagaaatggaagtctctccttctgcagagcagc

SEQ ID NO: 387 - DNA, 140 bp, Artificial Sequence
  Ex8 ssODN sequence 7 (70 nt each arm)
cgggaatctccaaacagtgagtcagatgttaagatgtcttgcttccacccccacaggcttactcgttccttgaaaggcagcttcggagtgacaaggaaagaaatggaagtctctccttctgcagagcagcttgtccaggt

SEQ ID NO: 388 - DNA, 160 bp, Artificial Sequence
  Ex8 ssODN sequence 8 (80 nt each arm)
cagattaagccgggaatctccaaacagtgagtcagatgttaagatgtcttgcttccacccccacaggcttactcgttccttgaaaggcagcttcggagtgacaaggaaagaaatggaagtctctccttctgcagagcagcttgtccaggtaggagcacag

SEQ ID NO: 389-390 - skipped (2 entries)

SEQ ID NO: 391 - DNA, 20 bp, Artificial Sequence
  Ex8 ssODN sequence 1a (10 nt each arm)
ctgcctttcaaggaacgagt

SEQ ID NO: 392 - DNA, 40 bp, Artificial Sequence
  Ex8 ssODN sequence 2a (20 nt each arm)
cactccgaagctgcctttcaaggaacgagtaagcctgtgg

SEQ ID NO: 393 - DNA, 60 bp, Artificial Sequence
  Ex8 ssODN sequence 3a (30 nt each arm)
ctttccttgtcactccgaagctgcctttcaaggaacgagtaagcctgtgggggtggaagc

SEQ ID NO: 394 - DNA, 80 bp, Artificial Sequence
  Ex8 ssODN sequence 4a (40 nt each arm)
acttccatttctttccttgtcactccgaagctgcctttcaaggaacgagtaagcctgtgggggtggaagcaagacatctt

SEQ ID NO: 395 - DNA, 100 bp, Artificial Sequence
  Ex8 ssODN sequence 5a (50 nt each arm)
agaaggagagacttccatttctttccttgtcactccgaagctgcctttcaaggaacgagtaagcctgtgggggtggaagcaagacatcttaacatctgac

SEQ ID NO: 396 - DNA, 120 bp, Artificial Sequence
  Ex8 ssODN sequence 6a (60 nt each arm)
gctgctctgcagaaggagagacttccatttctttccttgtcactccgaagctgcctttcaaggaacgagtaagcctgtgggggtggaagcaagacatcttaacatctgactcactgtttg

SEQ ID NO: 397 - DNA, 140 bp, Artificial Sequence
  Ex8 ssODN sequence 7a (70 nt each arm)
acctggacaagctgctctgcagaaggagagacttccatttctttccttgtcactccgaagctgcctttcaaggaacgagtaagcctgtgggggtggaagcaagacatcttaacatctgactcactgtttggagattcccg

SEQ ID NO: 398 - DNA, 160 bp, Artificial Sequence
  Ex8 ssODN sequence 8a (80 nt each arm)
ctgtgctcctacctggacaagctgctctgcagaaggagagacttccatttctttccttgtcactccgaagctgcctttcaaggaacgagtaagcctgtgggggtggaagcaagacatcttaacatctgactcactgtttggagattcccggcttaatctg

SEQ ID NO: 399 - skipped

SEQ ID NO: 400 - DNA, 224 bp, Artificial Sequence
  Human HTT Exon 31
ttgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcggg

SEQ ID NO: 401 - DNA, 820 bp, Artificial Sequence
  Human HTT Exon 31-containing segment
tgtggagggcagaggacaccagaagctcctctgcaacctcgctagttaacttccagtccctcggagtttctgtttagaatgctcaatctcatttagaattgcaaggaaacccaaaacgcctatttaaggtacaaacagcacttcatacaatatctcatgaggtattaatagtgattcacaggaagaatttcacgctgtgagtctttgctaacatatccagttatttacagatggatttgatatttgtgtgggagattcttaaaagtgttgttcacgccacattgttgatgcctcatttttttcactgtagttgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcggggtaacagttgtggcaagaatgctgtcgttggtggaagcacgaaagagcaagcaggaaatactttgtaaaagaataaaaacgaaaaatgttagcgaacatcttctaatagtctgctgtattcagagaactctaggagatatatatggttgatgcaaagatgatttaaggcatagcccggccttccaagaagtgtgtggccagtgagtgagatgggcttgggacttacacatctcagaggtgggggtagaggaggaggaacactgagtgggctgagaagcagccagct

SEQ ID NO: 402 - DNA, 284 bp, Artificial Sequence
  Human HTT Exon 31 plus 30 nt each end
attgttgatgcctcatttttttcactgtagttgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcggggtaacagttgtggcaagaatgctgtcgttg

SEQ ID NO: 403-404 - skipped (2 entries)

SEQ ID NO: 405 - DNA, 224 bp, Artificial Sequence
  Reverse complementary sequence of Human HTT Exon 31 sequence
cccgaggtgtcgttctcctgctccgcctgcaccatgttcctcaggctggcgtcagcgagggcctgggtgaagtgggtgtacggggccatgaagcagtagtggtacaagcctggcctcacactggaggagccaaggcgctgtgctcggccttgtgacttgctggggttggaagataagccatcaaactgggaggccaagtttgtgccaaagagagtcttcaacaa

SEQ ID NO: 406 - DNA, 820 bp, Artificial Sequence
  Reverse complementary sequence of Human HTT Exon 31-containing segment sequence
agctggctgcttctcagcccactcagtgttcctcctcctctacccccacctctgagatgtgtaagtcccaagcccatctcactcactggccacacacttcttggaaggccgggctatgccttaaatcatctttgcatcaaccatatatatctcctagagttctctgaatacagcagactattagaagatgttcgctaacatttttcgtttttattcttttacaaagtatttcctgcttgctctttcgtgcttccaccaacgacagcattcttgccacaactgttaccccgaggtgtcgttctcctgctccgcctgcaccatgttcctcaggctggcgtcagcgagggcctgggtgaagtgggtgtacggggccatgaagcagtagtggtacaagcctggcctcacactggaggagccaaggcgctgtgctcggccttgtgacttgctggggttggaagataagccatcaaactgggaggccaagtttgtgccaaagagagtcttcaacaactacagtgaaaaaaatgaggcatcaacaatgtggcgtgaacaacacttttaagaatctcccacacaaatatcaaatccatctgtaaataactggatatgttagcaaagactcacagcgtgaaattcttcctgtgaatcactattaatacctcatgagatattgtatgaagtgctgtttgtaccttaaataggcgttttgggtttccttgcaattctaaatgagattgagcattctaaacagaaactccgagggactggaagttaactagcgaggttgcagaggagcttctggtgtcctctgccctccaca

SEQ ID NO: 407 - DNA, 284 bp, Artificial Sequence
  Reverse complementary sequence of Human HTT Exon 31 plus 30 nt each end sequence
caacgacagcattcttgccacaactgttaccccgaggtgtcgttctcctgctccgcctgcaccatgttcctcaggctggcgtcagcgagggcctgggtgaagtgggtgtacggggccatgaagcagtagtggtacaagcctggcctcacactggaggagccaaggcgctgtgctcggccttgtgacttgctggggttggaagataagccatcaaactgggaggccaagtttgtgccaaagagagtcttcaacaactacagtgaaaaaaatgaggcatcaacaat

SEQ ID NO: 408-409 - skipped (2 entries)

SEQ ID NO: 410 - DNA, 20 bp, Artificial Sequence
  Ex31-1 gRNA targeting sequence (20nt)
agataagccatcaaactggg

SEQ ID NO: 411 - DNA, 19 bp, Artificial Sequence
  Ex31-1 gRNA targeting sequence (19nt)
gataagccatcaaactggg

SEQ ID NO: 412 - DNA, 18 bp, Artificial Sequence
  Ex31-1 gRNA targeting sequence (18nt)
ataagccatcaaactggg

SEQ ID NO: 413 - DNA, 17 bp, Artificial Sequence
  Ex31-1 gRNA targeting sequence (17nt)
taagccatcaaactggg

SEQ ID NO: 414-419 - skipped (6 entries)

SEQ ID NO: 420 - DNA, 20 bp, Artificial Sequence
  Ex31-2 gRNA targeting sequence (20nt)
gtacaagcctggcctcacac

SEQ ID NO: 421 - DNA, 19 bp, Artificial Sequence
  Ex31-2 gRNA targeting sequence (19nt)
tacaagcctggcctcacac

SEQ ID NO: 422 - DNA, 18 bp, Artificial Sequence
  Ex31-2 gRNA targeting sequence (18nt)
acaagcctggcctcacac

SEQ ID NO: 423 - DNA, 17 bp, Artificial Sequence
  Ex31-2 gRNA targeting sequence (17nt)
caagcctggcctcacac

SEQ ID NO: 424-429 - skipped (6 entries)

SEQ ID NO: 430 - DNA, 20 bp, Artificial Sequence
  Ex31-3 gRNA targeting sequence (20nt)
ccgcctgcaccatgttcctc

SEQ ID NO: 431 - DNA, 19 bp, Artificial Sequence
  Ex31-3 gRNA targeting sequence (19nt)
cgcctgcaccatgttcctc

SEQ ID NO: 432 - DNA, 18 bp, Artificial Sequence
  Ex31-3 gRNA targeting sequence (18nt)
gcctgcaccatgttcctc

SEQ ID NO: 433 - DNA, 17 bp, Artificial Sequence
  Ex31-3 gRNA targeting sequence (17nt)
cctgcaccatgttcctc

SEQ ID NO: 434-439 - skipped (6 entries)

SEQ ID NO: 440 - DNA, 20 bp, Artificial Sequence
  Ex31-2a gRNA targeting sequence (20nt)
gcctcacactggaggagcca

SEQ ID NO: 441 - DNA, 19 bp, Artificial Sequence
  Ex31-2a gRNA targeting sequence (19nt)
cctcacactggaggagcca

SEQ ID NO: 442 - DNA, 18 bp, Artificial Sequence
  Ex31-2a gRNA targeting sequence (18nt)
ctcacactggaggagcca

SEQ ID NO: 443 - DNA, 17 bp, Artificial Sequence
  Ex31-2a gRNA targeting sequence (17nt)
tcacactggaggagcca

SEQ ID NO: 444-449 - skipped (6 entries)

SEQ ID NO: 450 - DNA, 20 bp, Artificial Sequence
  Ex31-2b gRNA targeting sequence (20nt) containing 2 mismatches
gcctcacacttgaagagcca

SEQ ID NO: 451 - DNA, 19 bp, Artificial Sequence
  Ex31-2b gRNA targeting sequence (19nt) containing 2 mismatches
cctcacacttgaagagcca

SEQ ID NO: 452 - DNA, 18 bp, Artificial Sequence
  Ex31-2b gRNA targeting sequence (18nt) containing 2 mismatches
ctcacacttgaagagcca

SEQ ID NO: 453 - DNA, 17 bp, Artificial Sequence
  Ex31-2b gRNA targeting sequence (17nt) containing 2 mismatches
tcacacttgaagagcca

SEQ ID NO: 454-460 - skipped (7 entries)

SEQ ID NO: 461 - DNA, 10 bp, Artificial Sequence
  Ex31 ssODN 5′ homology arm (10 nt)
tggcctccca

SEQ ID NO: 462 - DNA, 20 bp, Artificial Sequence
  Ex31 ssODN 5′ homology arm (20 nt)
ggcacaaacttggcctccca

SEQ ID NO: 463 - DNA, 30 bp, Artificial Sequence
  Ex31 ssODN 5′ homology arm (30 nt)
gactctctttggcacaaacttggcctccca

SEQ ID NO: 464 - DNA, 40 bp, Artificial Sequence
  Ex31 ssODN 5′ homology arm (40 nt)
agttgttgaagactctctttggcacaaacttggcctccca

SEQ ID NO: 465 - DNA, 50 bp, Artificial Sequence
  Ex31 ssODN 5′ homology arm (50 nt)
ttttcactgtagttgttgaagactctctttggcacaaacttggcctccca

SEQ ID NO: 466 - DNA, 60 bp, Artificial Sequence
  Ex31 ssODN 5′ homology arm (60 nt)
tgcctcatttttttcactgtagttgttgaagactctctttggcacaaacttggcctccca

SEQ ID NO: 467 - DNA, 70 bp, Artificial Sequence
  Ex31 ssODN 5′ homology arm (70 nt)
acattgttgatgcctcatttttttcactgtagttgttgaagactctctttggcacaaacttggcctccca

SEQ ID NO: 468 - DNA, 80 bp, Artificial Sequence
  Ex31 ssODN 5′ homology arm (80 nt)
tgttcacgccacattgttgatgcctcatttttttcactgtagttgttgaagactctctttggcacaaacttggcctccca

SEQ ID NO: 469-470 - skipped (2 entries)

SEQ ID NO: 471 - DNA, 10 bp, Artificial Sequence
  Ex31 ssODN 3′ homology arm (10 nt)
aacatggtgc

SEQ ID NO: 472 - DNA, 20 bp, Artificial Sequence
  Ex31 ssODN 3′ homology arm (20 nt)
aacatggtgcaggcggagca

SEQ ID NO: 473 - DNA, 30 bp, Artificial Sequence
  Ex31 ssODN 3′ homology arm (30 nt)
aacatggtgcaggcggagcaggagaacgac

SEQ ID NO: 474 - DNA, 40 bp, Artificial Sequence
  Ex31 ssODN 3′ homology arm (40 nt)
aacatggtgcaggcggagcaggagaacgacacctcggggt

SEQ ID NO: 475 - DNA, 50 bp, Artificial Sequence
  Ex31 ssODN 3′ homology arm (50 nt)
aacatggtgcaggcggagcaggagaacgacacctcggggtaacagttgtg

SEQ ID NO: 476 - DNA, 60 bp, Artificial Sequence
  Ex31 ssODN 3′ homology arm (60 nt)
aacatggtgcaggcggagcaggagaacgacacctcggggtaacagttgtggcaagaatgc

SEQ ID NO: 477 - DNA, 70 bp, Artificial Sequence
  Ex31 ssODN 3′ homology arm (70 nt)
aacatggtgcaggcggagcaggagaacgacacctcggggtaacagttgtggcaagaatgctgtcgttggt

SEQ ID NO: 478 - DNA, 80 bp, Artificial Sequence
  Ex31 ssODN 3′ homology arm (80 nt)
aacatggtgcaggcggagcaggagaacgacacctcggggtaacagttgtggcaagaatgctgtcgttggtggaagcacga

SEQ ID NO: 479-480 - skipped (2 entries)

SEQ ID NO: 481 - DNA, 20 bp, Artificial Sequence
  Ex31 ssODN sequence 1 (10 nt each arm)
tggcctcccaaacatggtgc

SEQ ID NO: 482 - DNA, 40 bp, Artificial Sequence
  Ex31 ssODN sequence 2 (20 nt each arm)
ggcacaaacttggcctcccaaacatggtgcaggcggagca

SEQ ID NO: 483 - DNA, 60 bp, Artificial Sequence
  Ex31 ssODN sequence 3 (30 nt each arm)
gactctctttggcacaaacttggcctcccaaacatggtgcaggcggagcaggagaacgac

SEQ ID NO: 484 - DNA, 80 bp, Artificial Sequence
  Ex31 ssODN sequence 4 (40 nt each arm)
agttgttgaagactctctttggcacaaacttggcctcccaaacatggtgcaggcggagcaggagaacgacacctcggggt

SEQ ID NO: 485 - DNA, 100 bp, Artificial Sequence
  Ex31 ssODN sequence 5 (50 nt each arm)
ttttcactgtagttgttgaagactctctttggcacaaacttggcctcccaaacatggtgcaggcggagcaggagaacgacacctcggggtaacagttgtg

SEQ ID NO: 486 - DNA, 120 bp, Artificial Sequence
  Ex31 ssODN sequence 6 (60 nt each arm)
tgcctcatttttttcactgtagttgttgaagactctctttggcacaaacttggcctcccaaacatggtgcaggcggagcaggagaacgacacctcggggtaacagttgtggcaagaatgc

SEQ ID NO: 487 - DNA, 140 bp, Artificial Sequence
  Ex31 ssODN sequence 7 (70 nt each arm)
acattgttgatgcctcatttttttcactgtagttgttgaagactctctttggcacaaacttggcctcccaaacatggtgcaggcggagcaggagaacgacacctcggggtaacagttgtggcaagaatgctgtcgttggt

SEQ ID NO: 488 - DNA, 160 bp, Artificial Sequence
  Ex31 ssODN sequence 8 (80 nt each arm)
tgttcacgccacattgttgatgcctcatttttttcactgtagttgttgaagactctctttggcacaaacttggcctcccaaacatggtgcaggcggagcaggagaacgacacctcggggtaacagttgtggcaagaatgctgtcgttggtggaagcacga

SEQ ID NO: 489-490 - skipped (2 entries)

SEQ ID NO: 491 - DNA, 20 bp, Artificial Sequence
  Ex31 ssODN sequence 1a (10 nt each arm)
gcaccatgtttgggaggcca

SEQ ID NO: 492 - DNA, 40 bp, Artificial Sequence
  Ex31 ssODN sequence 2a (20 nt each arm)
tgctccgcctgcaccatgtttgggaggccaagtttgtgcc

SEQ ID NO: 493 - DNA, 60 bp, Artificial Sequence
  Ex31 ssODN sequence 3a (30 nt each arm)
gtcgttctcctgctccgcctgcaccatgtttgggaggccaagtttgtgccaaagagagtc

SEQ ID NO: 494 - DNA, 80 bp, Artificial Sequence
  Ex31 ssODN sequence 4a (40 nt each arm)
accccgaggtgtcgttctcctgctccgcctgcaccatgtttgggaggccaagtttgtgccaaagagagtcttcaacaact

SEQ ID NO: 495 - DNA, 100 bp, Artificial Sequence
  Ex31 ssODN sequence 5a (50 nt each arm)
cacaactgttaccccgaggtgtcgttctcctgctccgcctgcaccatgtttgggaggccaagtttgtgccaaagagagtcttcaacaactacagtgaaaa

SEQ ID NO: 496 - DNA, 120 bp, Artificial Sequence
  Ex31 ssODN sequence 6a (60 nt each arm)
gcattcttgccacaactgttaccccgaggtgtcgttctcctgctccgcctgcaccatgtttgggaggccaagtttgtgccaaagagagtcttcaacaactacagtgaaaaaaatgaggca

SEQ ID NO: 497 - DNA, 140 bp, Artificial Sequence
  Ex31 ssODN sequence 7a (70 nt each arm)
accaacgacagcattcttgccacaactgttaccccgaggtgtcgttctcctgctccgcctgcaccatgtttgggaggccaagtttgtgccaaagagagtcttcaacaactacagtgaaaaaaatgaggcatcaacaatgt

SEQ ID NO: 498 - DNA, 160 bp, Artificial Sequence
  Ex31 ssODN sequence 8a (80 nt each arm)
tcgtgcttccaccaacgacagcattcttgccacaactgttaccccgaggtgtcgttctcctgctccgcctgcaccatgtttgggaggccaagtttgtgccaaagagagtcttcaacaactacagtgaaaaaaatgaggcatcaacaatgtggcgtgaaca

SEQ ID NO: 499 - skipped

SEQ ID NO: 500 - DNA, 224 bp, Artificial Sequence
  Mouse Htt Exon 31
ctattgaagactctctttgggacaaacttagcctcacagtttgatggcttatcttccaaccccagcaagtctcagtgccgagctcagcgccttggctcttcaagtgtgaggcccggcttatatcactactgcttcatggcaccatacacgcacttcacacaggccttggctgacgcaagcctgaggaacatggtgcaggcggagcaggagcgtgatgcctcggg

SEQ ID NO: 501 - DNA, 525 bp, Artificial Sequence
  Mouse Htt Exon 31-containing segment
gcagattgtctatttccatcctgaccactgtctatcctaaccgctgtccactagctggcattgcagacaggcctgtgctgctcatctacattaaatttatattaagtgaagtactgatgtttccattctttcattctagctattgaagactctctttgggacaaacttagcctcacagtttgatggcttatcttccaaccccagcaagtctcagtgccgagctcagcgccttggctcttcaagtgtgaggcccggcttatatcactactgcttcatggcaccatacacgcacttcacacaggccttggctgacgcaagcctgaggaacatggtgcaggcggagcaggagcgtgatgcctcggggtaatatttatggtgcaagtttgtcactgatgatgcagacacgagttatgtgcagcccttccttgaaagtcattggctgagttatggtgtagtcagctatccagaggtgacaggctagagaggaggaacccaagctgcctgtgaagcataggttctaagttc

SEQ ID NO: 502-599 - skipped (98 entries)

SEQ ID NO: 600 - protein, 1368 aa, Artificial Sequence [sequence omitted]

SEQ ID NO: 601 - protein, 1393 aa, Artificial Sequence
  SpCas9 variant 1
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 602 - protein, 1393 aa, Artificial Sequence
  SpCas9 variant 2
MAPKKKRKVDKKYSIGLDIGTNSVGWAVITDEYKVPSKKFKVLGNTDRHSIKKNLIGALLFDSGETAEATRLKRTARRRYTRRKNRILYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIEEFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 603 - protein, 1399 aa, Artificial Sequence
  SpCas9 variant 3
MGSSHHHHHHHHENLYFQGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 604 - protein, 1396 aa, Artificial Sequence
  SpCas9 variant 4
MAHHHHHHGGSPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 605 - protein, 1386 aa, Artificial Sequence
  SpCas9 variant 5
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADHHHHHH

SEQ ID NO: 606 - protein, 1389 aa, Artificial Sequence
  SpCas9 variant 6
MAHHHHHHGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 607 - protein, 1399 aa, Artificial Sequence; identical to SEQ ID NO: 603
  SpCas9 variant 7

SEQ ID NO: 608 - protein, 1393 aa, Artificial Sequence
  SpCas9 variant 8
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNAVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 609 - protein, 1393 aa, Artificial Sequence
  SpCas9 variant 9
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEALYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 610 - protein, 1393 aa, Artificial Sequence
  SpCas9 variant 10
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 611 - protein, 1393 aa, Artificial Sequence
  SpCas9 variant 11
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH